(19) United States
(12) Patent Application Publication
(10) Pub. No.: US 2004/0047866 A1
(43) Pub. Date: Mar. 11, 2004

(54) Immunomodulatory methods using carbohydrate antigens

(75) Inventors: Donald A. Harn, Pembroke, MA (US); Luis Ignacio Terrazas, Saugus, MA (US)

Correspondence Address: LAHIVE & COCKFIELD, LLP., 28 STATE STREET, BOSTON, MA, 02109, US

(73) Assignee: President and Fellows of Harvard College, Cambridge, MA

(21) Appl. No.: 10/238,834

(22) Filed: Sep. 9, 2002

(51) Int. Cl. A61K 39/00; C08B 37/00

(52) U.S. Cl. 424/184.1; 536/53

(57) ABSTRACT

Methods for modulating immune responses, such as IgE responses and autoimmune responses, are provided. The methods involve contacting an cell with an agent comprising multivalent lacto-N-neotetraose (LNnT), which modulates an immune response. The methods are useful for enhancing production of non-specific polyclonal IgE, inhibiting production of antigen-specific IgE responses, inducing cytokine production, and stimulating proliferation of splenocytes. In a preferred embodiment, the invention provides methods for modulating an immune response to an antigen (e.g., an allergen) in vivo. Pharmaceutical compositions for modulating immune responses comprising the agents of the invention are also provided. The invention also provides for treatment or prevention of shock in a subject using multivalent LNnT and methods for treating or preventing an autoimmune disease in a subject using multivalent LNnT. Still further the invention provides for treatment of cancer in a subject using monovalent LNnT.

### RELATED APPLICATIONS

This application claims priority to U.S. Provisional Application Serial No. 60/197,059, filed Apr. 14, 2000, and PCT/US01/12365 (WO 01/78748), filed Apr. 16, 2001, the entire contents each of which are incorporated herein by reference.

### BACKGROUND OF THE INVENTION

Helminth infection is strongly associated with the production of large amounts of serum IgE in humans and experimental animals (King, C L. et al., (1993), J. Immunol. vol. 150 pp. 1873-1880; Ogilvie B M, Nature 1964 204. 91-92; Sadun, E H. et al. (1970) Exp. Parasitol. vol. 28 pp. 435-449). The majority of serum IgE in infected host is nonspecific and polyclonal (Dessaint, J P. et al. (1975) Clin. Exp. Immunol. vol. 20 pp. 427-436; Jarret, et al. (1976) Clin. Exp. Immunol. vol. 24 pp. 346-351). Such IgE has several roles in immunity against helminth parasites. Ag-specific IgE, especially anti-adult worm Ag, is involved with the resistance to reinfection (Hagan, P. et al. (1991) Nature vol. 349 pp. 243-245; Viana, I R C. et al. (1995) Parasite Immunol. vol. 17 pp. 297-304), and cell-mediated cytotoxicity against parasites (Capron, A. et al. (1980) Am. J. Trop. Med. Hyg. vol. 29 pp. 849-857; Gounni, A S. et al. (1994) Nature vol. 367 pp. 183-186; Cutts, L. et al. (1997) Parasite Immunol. vol. 19 pp. 91-102). On the other hand, non-specific, polyclonal IgE seems to be beneficial for both host and parasites to reduce the risk of the potentially lethal anaphylactic reaction to parasitic antigens by saturating the available Fc_{ε}Rs on effector cells, although IgE plays a detrimental role for the host in primary infection of schistosomiasis (Hagan, P. (1993) Parasite Immunol. vol. 15 p. 14; Pritchard, D I. (1993) Parasite Immunol. vol. 15 pp. 5-9; Amiri, P. et al. (1993) J. Exp. Med. vol 0.180 pp. 43-51).

Several allergic components in schistosomal Ag have been reported to react with IgE from humans and rodents at various stages of infections. (Damonneville, M. et al. (1984) Int. Arch. Allergy Appl. Immunol. vol. 73 pp. 248-255; Owhashi, M. et al. (1986) Int. Arch. Allergy appl. Immunol. vol. 81 pp. 129-135). However little is known about the molecules that induce polyclonal IgE production from host. Toxocara canis adult worm antigen has B cell mitogenic activity to induce proliferation, IgG and IgE production although the allergenic molecule remains undefined (Wang, M Q. et al. (1995) Parasite Immunol. vol. 17 pp. 609-615. Yamashita, U. et al. (1993) Jap. J. Parasitol. vol. 42 pp. 211-219). Ascaris body fluid also contains a B cell mitogen, however, this mitogen needs the help of IL4 delivered by the other factors to induce polyclonal IgE production (McGibbon, A M. et al. (1990) Mol. Biochem. Parasitol. vol. 39 pp. 163-172; Lee, T D G. et al. (1995) J. Allergy Clin. Immunol. vol. 95 pp. 124-1254. Lee, T D G. et al. (1993) Int. Arch. Allergy Immunol. vol. 102 pp. 185-190). Two recombinant filarial proteins are capable of inducing polyclonal IgE production in vitro, however, they also induce Ag-specific IgE (Garrud, O. et al. (1995) J. Immunol. vol. 155 pp. 131-1325).

Schistosoma mansoni synthesize glycoproteins containing polylactosamine sugars (Srivastan, J. et al. (1992) J. Biol. Chem. vol. 267 pp. 14730-14737; Van Dam, G J. et al. (1994) Eur. J. Biochem. vol. 225 pp. 467-482). Lacto-N-fucopentaose III (LNFIII) found on adult worm and egg of S. mansoni has been found to be an antigenic determinant (KO, Al. et al. (1990) Proc. Natl. Acad. Sci. USA vol. 87 pp. 4159-4163). LNFIII stimulates splenic B cells from parasite-infected mice to proliferate and produce IL-10, a cytokine that downregulates Th1 immune responses (W Velupillai, P. et al. Proc. Natl. Acad. Sci. USA vol. 91 pp. 18-22; Palanivel, V. et al. (1996) Exp. Parasitol. vol. 84 pp. 168-177; Velupillai, P. et al. (1997) J. Immunol. vol. 158 pp. 338-344). In addition, this sugar has been found to have an adjuvant effect in inducing Th2 immune reaction in both Ab and cytokine production against protein with which the sugar is physically conjugated.

The course of many autoimmune disease states is influenced by whether a predominant Th1 response or Th2 response is mounted. For instance, patients with rheumatoid arthritis have predominantly Th1 cells in synovial tissue (Simon et al. (1994) Proc. Natl. Acad. Sci. USA 91:8562-8566) and experimental autoimmune encephalomyelitis (EAE) can be induced by autoreactive Th1 cells (Kuchroo et al. (1993) J. Immunol. 151:4371-4381). Type 1 insulin-dependent diabetes mellitus (IDDM) is characterized by the destruction of the pancreatic beta cells by autoreactive T lymphocytes (Gross, D. J., et al. (2001) Int. Immunopharmacol. 1:1131-1139). Similarly, psoriasis has been associated with a variety of autoantibodies, including antinuclear antibodies, antibodies to small nuclear and cytoplasmic ribonucleoproteins, and antibodies to epidermal cells (Reeves, W. H. (1991) Semin Dermatol. 10:217-224). Finally, inflammatory bowel disease (IBD) is associated with immunological overreaction (hypersensitivity) to commensal gut bacteria (Brandtzaeg, P. (2001) Acta Odontol Scand 59:235-243).

Interestingly, a low incidence of autoimmune disease has been associated with populations endemic for intestinal parasite. The low incidence of the Th1-mediated diseases is thought to be due to the ability of helminth parasites to drive polarized Th2-type responses or, in some individuals, induce immune anergy, thus suppressing development of Th1-type disease (Pearce, E. J., et al. (1991) J. Exp. Med. 173:159-166; Grzych, J-M., et al. (1991) J. Immunol. 141:1322-1327; Kullberg, M. C., et al. (1992)J. Immunol. 148:3264-3270). One such parasitic helminth, Schistosoma mansoni, synthesize glycoproteins containing polylactosamine sugars (Srivastan, J. et al. (1992) J. Biol. Chem. vol. 267 pp. 14730-14737; Van Dam, G J. et al. (1994) Eur. J. Biochem. vol. 225 pp. 467-482). For example, the sugar lacto-N-fucopentaose III (LNFPIII), found on adult worms and eggs of S. mansoni, has been found to be an antigenic determinant (KO, A I. et al. (1990) Proc. Natl. Acad. Sci. USA vol. 87 pp. 4159-4163). Specifically, LNFPIII stimulates splenic B cells from parasite-infected mice to proliferate and produce IL-10, a cytokine that downregulates Th1 immune responses (W Velupillai, P. et al. Proc. Natl. Acad. Sci. USA vol. 91 pp. 18-22; Palanivel, V. et al. (1996) Exp. Parasitol. vol. 84 pp. 168-177; Velupillai, P. et al. (1997) J. Immunol. vol. 158 pp. 338-344.

Additional insight into molecules which bias the immune response towards specific cytokine profiles will be important in developing methods of regulating immune responses. In particular, identification of molecules which induce polyclonal IgE synthesis will be of tremendous benefit in treating allergy. In addition, molecules that modulate immune responses will be especially useful for the treatment of many autoimmune disorders, and as vaccine adjuvants that direct the immune response to the vaccine in a desired manner.

### SUMMARY OF THE INVENTION

The instant invention provides novel methods of regulating the immune response. The invention is based, at least in part, on the discovery of the functional characteristics of LNnT, a non-fucosylated homologue of LNFIII, that is converted to LNFIII by α1-3 fucosyltransferase in adult worms. Multivalent LNnT induces polyclonal IgE production and cytokine production (e.g., IL-10, IL-5, IL-4, IL-13 and TGF-β) from mice by IP inoculation. This molecule can be used to modulate the IgE response, not only to parasite antigens, but to environmental allergens by saturating Fcε receptors on the effector cells.

The invention further pertains to other uses of LNnT, either in a multivalent form or as a free sugar (i.e., monovalent form), in the modulation of immune responses. For example, multivalent LNnT (e.g., LNnT conjugated to a carrier) has been found to render spleen cells less responsive to lipopolysaccharide (LPS) and Con-A (e.g., as measured by spleen cell proliferation and the production of the Th1-associated cytokines interleukin-12 (IL-12), interleukin-18 (IL-18) and interferon-gamma (IFN-γ)). Accordingly, multivalent LNnT can be used to protect a subject from the effects of shock (e.g., toxic shock), for example as a pretreatment in subjects susceptible to shock (e.g., surgery patients at risk for shock).

Moreover, the ability of multivalent LNnT to downmodulate Th1-associated cytokines allows for use of such agents in any clinical setting in which it is desirable to downmodulate type 1 immune responses, such as in Th1-associated autoimmune diseases (including inflammatory bowel disease, Type 1 diabetes and rheumatoid arthritis). For example, multivalent conjugates of LNnT, such as those described herein, can be used to treat a Th1-associated autoimmune disease, and/or to prevent an Th1-associated autoimmune disease (e.g., immunization).

Furthermore, multivalent LNnT has been found to induce a population of suppressor cells that are Gr1+, CD11b+. These suppressor cells, which also can be induced by sugars expressed on tumor cells, are able to suppress T cell proliferative responses and furthermore can make cytokines that may downregulate immune responses or that may promote angiogenesis (such as TGFβ), Thus, in the context of tumor treatment, it would be desirable to inhibit the induction of this population of suppressor cells. The invention provides for the use of free, non-crosslinked, monovalent LNnT-containing oligosaccharides to inhibit the generation of these Gr1+, CD11b+ suppressor cells, for example in the treatment of cancer. In contrast, in other clinical settings it may be desirable to induce this Gr1+, CD11b+ suppressor cell population, for example in situations where one wants to downmodulate immune responses, such as in autoimmune diseases (e.g., inflammatory bowel disease, diabetes, rheumatoid arthritis, allergic asthma, multiple sclerosis). Accordingly, other embodiments of the invention provide for use of multivalent LNnT to induce (e.g., stimulate, recruit) the generation of Gr1+, CD11b+ suppressor cells, for example in the treatment of Th1-associated autoimmune diseases, and other contexts wherein suppression of the immune system is desirable (e.g., transplantation, allergy)

### BRIEF DESCRIPTION OF THE DRAWINGS

FIGS. 1a-1c: Serum Ig production in BALB/C mice following IP immunization. FIG. 1(a): Female BALB/C mice were IP immunized with saline, HSA, Le^{y}-HSA, LNnT-HSA or HSA-Alum. All the antigens were inoculated at the dose of 10 μg protein weight of HSA. 6 and 5 days following first (dotted bar) and second (open bar) boosting immunization, respectively, blood samples were taken and serum total IgE was measured. Results shown are the mean±1 SE of four individual serum. FIG. 1(b): Course of serum total IgE following IP immunization. BALB/C mice were IP immunized with saline (open square), HSA (10 μg; open triangle) or LNnT-HSA (10 μg of HSA; closed circle) at day 0. Two weeks later, the boosting immunization was followed in a same fashion. Blood was taken at day 10, 20, 27, 41, and 69, and serum total IgE was determined. Results shown are the mean±1 SE of four individual serum. FIG. 1(c): Serum IgG isotypes 5 days following third IP immunization with saline (dotted bar), HSA (open bar), or LNnT-HSA (bar with slanted lines). Results shown are the mean±1 SE of four individual serum. All the results are representative of three experiments.

FIGS. 2a-2d Antigen-specific Ab production following IP immunization with multivalent sugars. Female BALB/C mice were immunized and bled as described in FIG. 1. Following first (dotted bar) and second (open bar) immunization, HSA-specific IgE (FIG. 2a), HSA-specific IgG (FIG. 2b), LNnT-HSA-specific IgE (FIG. 2c), and LNnT-HSA-specific IgG (FIG. 2d) were measured by ELISA as described in materials and methods. Specific IgE were determined as the absorbance at 450 nm of sera diluted 4 times. Specific IgG were determined as the endpoint titer. Results shown are the mean±1 SE of four individual serum. Results are representative of three experiments.

FIGS. 3a-3d: Serum total IgE in CBA/J (FIG. 3a) and C57BL/6 (FIG. 3b) mice following IP immunization with saline, HSA or LNnT-HSA. 6 and 5 days following first (dotted bar) and second (open bar) boosting immunization, respectively, blood samples were taken and serum total IgE were determined. Results shown are the mean±1 SE of four individual serum. FIG. 3c: Course of serum total IgE following SC immunization. BALB/C mice were SC immunized with saline (open square), HSA (10 μg; open triangle) or LNnT-HSA (10 μg of HSA; closed circle) at day 0. Two weeks later, the boosting immunization was followed in a same fashion. Blood was taken at day 10, 20, 27, 41, and 69, and serum total IgE was determined. Results shown are the mean±1 SE of four individual serum. FIG. 3D: Serum total IgE in CBA/CaJ (open bar) and CBA/CaJ xid (hatched bar) following second boosting IP immunization with saline, HSA or LNnT-HSA. Results shown are the mean±1 SE of four individual serum. Results are representative of two experiments.

FIG. 4 Proliferative responses of splenocytes. BALB/C mice were IP immunized with saline, HSA (10 μg) or LNnT-HSA (10 μg weight of HSA). 2 and 3 weeks later, boosting immunization was done in the same fashion. 5 days following the final immunization, spleens were removed. 2.5×10⁶ splenocytes were stimulated with ConA (2 μg/ml; dotted bar), HSA (10 μg/ml; hatched bar), LNnT-HSA (10 μg/ml of HSA; closed bar) or no restimulation (open bar). Results shown are the mean cpm (experimental-medium control)±1 SE of four individual mice per group. Results are representative of three experiments.

FIGS. 5a-5h: B7-1 and B7-2 expression on B220+ cells. Splenocytes from mice IP immunized with saline (FIGS. 5a, 5d), HSA (FIGS. 5b, 5e), or LNnT-HSA (FIGS. 5c, 5f) were incubated for 24 hrs without additional stimulants. Cell pellets were stained with PE-conjugated mAb against either B7-1 (FIGS. 5a, 5b, 5c) or B7-2 (FIGS. 5d, 5e, 5f). Numbers expressed in upper right quadrant show the mean±1 SE of percentage of cells expressing both B220 and B7 molecules from six individual sample. FIGS. 5g, 5h: Effect of the duration of culture incubation on B7 expression in mice IP immunized with saline (open square), HSA (open triangle) or LNnT-HSA (closed circle). Percentage of B220+ cells expressing B7-1 (FIG. 5g) or B7-2 (FIG. 5h) were monitored at preincubation, 24, 72, and 120 hrs postincubation without restimulation. Results were representative for two experiments.

FIG. 6: Levels of total IgE (ng/ml) in mice treated two (open bars) or three (dotted bars) times intraperitoneally with the LNnT-dextran conjugate LNnT35.

FIG. 7: Levels of total IgE (ng/ml) in mice treated first with RMPI (saline), ovalbumin, the LNnT-dextran conjugate LNnT45, or dextran followed by booster treatment with ovalbumin.

FIG. 8: Levels of ovalbumin-specific IgE in mice treated first with RMPI (saline), ovalbumin, the LNnT-dextran conjugate LNnT45, or dextran followed by booster treatment with ovalbumin.

FIG. 9: Levels of total IgE (ng/ml) over time (up to 70 days post-immunization) in mice treated with either saline, HSA or LNnT-HSA.

FIGS. 10a-10c: Levels of Th2-type cytokine production by total spleen cells of mice immunized with vehicle (dextran) or LNnT-dextran conjugate. FIG. 10a shows IL-13 levels (pg/ml). FIG. 10b shows IL-4 levels (pg/ml). FIG. 10c shows IL-10 levels (pg/ml).

FIG. 11: Level of production of the Th1-type cytokine interferon-gamma (pg/ml) by total spleen cells of mice immunized with vehicle (dextran) or LNnT-dextran conjugate.

FIG. 12: Proliferative responses of total spleen cells from mice treated with vehicle (dextran) or LNnT-dextran conjugate in vivo, followed by stimulation of harvested spleen cells with LPS in vitro.

FIG. 13: Level of production of the Th1-type cytokine interferon-gamma (pg/ml) by spleen cells from mice treated with vehicle (dextran) or LNnT-dextran conjugate in vivo, followed by stimulation of harvested spleen cells with ConA in vitro.

FIG. 14: Level of production of the Th1-type cytokine IL-12 (pg/ml) by spleen cells from mice treated with vehicle (dextran) or LNnT-dextran conjugate in vivo, followed by stimulation of harvested spleen cells with LPS or LNnT-dextran in vitro.

FIG. 15: Level of production of the Th2-type cytokine IL-13 (pg/ml) by spleen cells from mice treated with vehicle (dextran) or LNnT-dextran conjugate in vivo, followed by stimulation of harvested spleen cells with ConA or LNnT-dextran in vitro.

FIG. 16: Level of production of the Th2-type cytokine IL-13 (pg/ml) by CD4+ cells from mice treated with vehicle (dextran) or LNnT-dextran conjugate in vivo, followed by stimulation of harvested spleen cells with ConA in vitro.

FIG. 17: Proliferative responses of naïve spleen cells stimulated with anti-CD3 in the presence of peritoneal exudate cells (PECs) from mice treated with vehicle (Dex) or LNnT-dex conjugate (LNnT) or in the presence of PECs from LNnT-dex injected mice which had the Gr1+ cells removed from the PEC population (LNnT(−)).

FIG. 18: Proliferative responses of CD4+ cells from Balb/c mice stimulated with anti-CD3 in the presence of peritoneal exudate cells (PECs) from mice treated with vehicle (Dex), saline (control), or LNnT-dex conjugate (LNnT) or Gr1+ enriched PECs (Gr1+) from LNnT-dex injected mice.

FIG. 19: LNnT-Dex injection recruits greater numbers of peritoneal cells expressing Gr-1+/CD11b+/F4/80+ surface markers. (a) Number of PECs recruited at different times after a single LNnT-Dex injection. (b) FACS analysis of PECs recruited by LNnT-Dex, dextran or saline. Mice injected with LNnT-Dex showed an increase in the markers for Gr-1/CD11b and F4/80. No changes are observed in B7-2, CD40 and CD11b between the different groups. PECs were obtained at different times post-sugar injection, analysis was performed in individual mice, 4 mice per group. Data are representative of five separate, independently performed experiments with BALB/c and C57BL/6 strains.

FIG. 20: PECs recruited by LNnT-Dex inhibit the proliferative response of naive CD4 cells stimulated with plate-bound anti-CD3/CD28 antibodies. (a) PECs from C57BL/6 mice were obtained 2 h or (b) from BALB/c mice 18 h post-injection of LNnT-Dex or dextran, co-cultured at different ratios with 1×10⁵ previously stimulated naïve CD4⁺ cells. 72 h later ³H-Thymidine was added to the cultures and after 18 h cells were harvested and processed for radioactivity uptake. Individual mice were assayed and data are shown as mean±SE. Results are representative of four independent experiments performed with BALB/c and C57BL/6 strain of mice at both time points. *p<0.05.

FIG. 21: Depletion of Gr-1⁺ cells from PECs recruited by LNnT-Dex restores the proliferative response to anti-CD3/CD28 antibodies in naive CD4⁺ cells. PECs recruited by LNnT-Dex or dextran were harvested, pooled and depleted of Gr1⁺ cells by MACS and added in a ratio 1:2 to naive CD4⁺ cells previously stimulated with anti-CD3/CD28. Cells were co-cultured for 72 h and incorporation of ³H-Thymidine was measured. Results are the average of triplicates and are representative of three separate experiments performed in BALB/c and C57BL/6 mice. *p<0.05.

FIG. 22: Proliferative inhibition by PECs recruited by LNnT-Dex can be mediated by both cell to cell contact and by soluble factors. PECs harvested at 2 h or 18 h post-sugar injection either fixed with 0.5% of paraformaldehyde before being directly co-cultured with CD4 naive cells (a) or placed in a separate transwell plate separated by a 0.4 μm membrane (b). PECs were obtained from individual mice and triplicate wells set up. Proliferation was measured by ³H-Thymidine uptake. Data are shown as mean±SE from 4 animals per group and are representative of three independent experiments. *p<0.05.

FIG. 23: PECs recruited by LNnT-Dex modify the cytokine profile of CD4⁺ cells stimulated with anti-CD3/CD28 antibodies. PECs from animals injected with LNnT-Dex or dextran were co-cultured with CD4⁺ naive cells, after 72 h the IFN-γ (a) and IL-13 production (b) were assayed by ELISA.

FIG. 24: Suppressive PECs recruited by LNnT-Dex commit CD4 cells to produce more IL-13 in a secondary stimulation. CD4 naive cells were previously stimulated with plate bound anti-CD3/CD28 and 3 h later PECs (ratio 1:4) recruited by LNnT-Dex or dextran were added to cultures in the absence (a-b) or presence (c-d) of IL-12 (50 ng/ml). After 3 days, primed CD4 cells were washed, re-purified, rested for 3 more days and re-stimulated with anti-CD3/CD28 antibodies. 24 h after secondary stimulation supernatants were assayed for IFN-γ (a-c) and IL-13 (c-d) production. Data are representative of three experiments performed in BALB/c mice, and show mean±SE of triplicate cultures of 4 mice assayed individually. *p<0.05.

FIG. 25: IL-10 is involved in the soluble-mediated suppressive activity of PECs recruited by LNnT-Dex. Co-cultures of PECs and CD4⁺ (1:4 ratio) were performed as described in material and methods and neutralizing monoclonal anti-IL-10 or anti-TGF-β antibodies were added to the in vitro co-cultures. After 72 h proliferation was measured by ³H-Thymidine uptake. Data are representative of three experiments performed in BALB/c mice, and show mean±SE of triplicate cultures of 4 mice assayed individually. *p<0.05 compared to PECs-dextran.

FIG. 26: depicts dot blots obtained on a FACS Calibur that sow the percentage cells double stained for IFNγ or TNF-α and CD4. Spleens from mice sacrificed at day 16 post cell transfer were pooled, T cells purified and stimulated with αCD3 and αCD28 overnight. T cells were analyzed for intracellular cytokines after staining for surface CD4 Cy for reconstituted SCID mice and for BALB/c controls. Data are representative of two separate experiments using 4 mice per group.

FIG. 27: a and b are plots that represent a clinical index for (a) colon thickening and (b) cellular inflammation using a scale of 0-3, where 3 indicates the highest level of colon pathology. A bar (−) represents the mean score over 4 mice in each group in a single experiment. The plots are representative of colon scores for 4 experiments (represented by the symbols ▪, ^{▪}, ▴, and ♦) for mice treated with either glycoconjugate or dextran control.

FIGS. 28a and b depict dot plots in which CCR5 expression on spleen or lymph node (LN) cells from mice treated with (a) LNnT35-dex, 150 μg/mouse/week starting at day 2 post cell transfer, and (b) LNnT25-dex treatment 150 μg/mouse/week starting treatment at day 16 post cell transfer. SCID mice were reconstituted with sorted T cells, placed in groups of 4, and treated with LNnT-dex glycoconjugate or dextran. On sacrifice, spleen or mesenteric LN and Peyers' patches cells from each group were pooled and surface stained for CD4 and CCR5. Data was collected on a FACS Calibur.

FIG. 29 depicts dot plots of β7 integrin expression on spleen or LN cells from groups of dextran treated or glycoconjugate treated (LNnT35-dex) reconstituted SCID mice. Spleen or Peyers' patches and mesenteric lymph node cells were harvested and stained for integrin α4β7 and αIELβy. Lowered integrin levels were observed only in lymph node T cells from glycoconjugate treated mice.

FIG. 30 is a bar graph of cytokine changes in reconstituted SCID mice T cells. SCID mice in groups of 4 were treated with glycoconjugate. On sacrifice, purified spleen CD4 T cells pooled within each group were stimulated for 3 days with αCD3 and αCD28. Supernatants were analyzed for IL-4 by ELISA. Data is representative of 2 separate experiments from mice treated with LNnT25-dex or control. Mean of duplicate wells +/−standard deviation is shown. ** indicates significance at P<0.01 by Unpaired Students' T Test.

### DETAILED DESCRIPTION OF THE INVENTION

This invention provides immunomodulatory methods in which a cell (e.g., a human immune cell) is contacted with an agent which modulates an immune response (e.g., non-specific polyclonal IgE production, immune cell mitogenesis, or production by the cell of one or more cytokines). The invention is based, at least in part, on the discovery that when animals are immunized with multivalent lacto-N-neotetraose (LNnT), a carbohydrate that is putatively expressed on helminth parasite Schistosoma mansosi, both BALB/C and CBA/J mice produced significantly higher amounts of total serum IgE following two intraperitoneal (IP) immunizations with multivalent LNnT conjugated to human serum albumin (LNnT-HSA) compared to groups immunized with saline or HSA alone. Interestingly, no specific IgE against the carbohydrate or the carrier protein was detected in ELISA, suggesting an induction of polyclonal nonspecific IgE production in vivo by multivalent LNnT. Moreover, this neo-glycoprotein did not promote significant production of IgG against either the carbohydrate or the carrier protein. C57BL/6 mice only showed the elevation of serum total IgE after three times immunization with LNnT-HSA, reflecting strain-dependent reactions against LNnT-HSA. Spleen cells from mice IP immunized with LNnT-HSA produced in vitro significant amount of IL-4, IL-5, and IL-10 as well as IL-2 and IFN-γ compared to controls. In addition, cultured B220+ cells had increased expression of B7-2 (CD86) but not B7-1 (CD80), suggesting that B7-2 expression is strongly associated with polyclonal production of IgE. Further, IL-4 gene deficient BALB/C mice did not produce polyclonal IgE following IP immunization with LNnT-HSA. Spleen cells from these mice produced lower but significant amounts of IL-5 and IL-10, and same amounts of IFN-γ compared to the wild type, demonstrating that IL-4 is critical for promoting polyclonal IgE production induced by the multivalent-carbohydrate naturally expressed on helminth parasite.

Additional experiments demonstrated that LNnT conjugated to dextran also stimulates polyclonal IgE responses and that pretreatment with multivalent LNnT (e.g., LNnT conjugated to dextran), prior to immunization with an antigen, inhibits the production of antigen-specific IgE responses. The enhanced levels of polyclonal IgE stimulated by multivalent LNnT are persistent (e.g., sustained for at least 70 days). In addition to IL-4, the production of Th2-type cytokines IL-10 and IL-13 is stimulated by treatment with LNnT conjugated to dextran, whereas levels of the Th1-type cytokine interferon gamma are inhibited by treatment with LNnT conjugated to dextran.

Thus, the methods of the invention allow for IgE production to be modulated (e.g., stimulation of non-specific IgE and/or inhibition of antigen-specific IgE), as well as allowing for modulation of cytokine production. Accordingly, the immunomodulatory methods of the invention allow for an immune response to be biased towards a specific cytokine secretion profile, for example, a Th2 response. The ability to influence the production of non-specific, polyclonal IgE using the immunomodulatory methods of the invention can be used in the prevention of detrimental host reaction against parasite infection and is further applicable to the protection against environmental allergens by saturating FcεRs on effector cells. Moreover, the ability to influence the development of a Th2 response using the immunomodulatory methods of the invention is applicable to the treatment of a wide variety of disorders, including cancer, infectious diseases (e.g., HIV and tuberculosis), allergies and autoimmune diseases.

Another aspect of the invention pertains to use of multivalent LNnT in the treatment or prevention of shock. As demonstrated in Example 3, administration of multivalent LNnT (e.g., LNnT conjugated to dextran) renders spleen cells less responsive to LPS, as measured by proliferative responses and production of type 1 cytokines such as IL-12 and IFN-γ. Thus, multivalent LNnT treatment can be used to inhibit the shock response in a patient, either in a patient suffering from shock or, more preferably, in a patient at risk of (susceptible to) shock, such as a surgery patient who is susceptible to shock. For at risk patients, these patients can be pretreated with a multivalent LNnT composition of the invention to render them less susceptible to shock.

Yet another aspect of the invention pertains to methods for inhibiting the induction of suppressor cells, in particular a population of Gr1+, CD11b+ suppressor cells, by blocking their induction using monovalent (free, unconjugated) LNnT. As demonstrated in Example 4, administration of multivalent LNnT induces this suppressor cell population, which is capable of inhibiting T cell proliferative responses. This suppressor cell population may be induced in a clinical setting in a patient by tumor cells that express multivalent LNnT, and the induction of such a suppressor population could promote tumor growth and expansion, by suppression of immune responses against the tumor and/or by production by the suppressor population of factors (such as TGF-β) that promote tumor angiogenesis. Accordingly, inhibition of the induction of this suppressor population can be used in the treatment of cancer, by administration of monovalent LNnT to thereby competitively block the induction of the suppressor population.

In yet another aspect pertains to the use of multivalent LNnT for the treatment or prevention of a Th1-associated autoimmune disease. As described in Examples 6-8, administration of multivalent LNnT can treat or prevent the onset of Th 1-associated autoimmune disorders, such as Type I diabetes, rheumatoid arthritis, psoriasis and inflammatory bowel disease.

In order that the present invention may be more readily understood, certain terms are first defined. Standard abbreviations for sugars are used herein. For example, the following abbreviations are used throughout the text: “Gal” for galactose; “Glc” to glucose; “GlcNAc” for N-acetylglucosamine; “GalNAc” for N-acetylgalactoseamine; “Fuc” for fucose; “NAN or NeuAc” for N-acetylneuraminic acid. (e.g., see Rules of Carbohydrate Nomenclature in “Compendium of Macromolecular Nomenclature,” Blackwell Scientific Publications, Oxford (1991); and http//www.chem.qmw.ac.uk/iupac/2carb/).

As used herein, the term “lacto-N-neotetraose” (“LNnT”) is intended to refer to a polylactosamine sugar having the core sequence {Gal(β1-4)GlcNac(β1-3)Gal(β1-4)Glc}. This sugar is a non-fucosylated homologue of lacto-N-fucopentaose III {Gal(β1-4)[Fuc(β1-3]GlcNac(β1-3)Gal(β1-4)Glc} which is found at least on the parasite Schistosoma mansoni. As used herein, an “LNnT-containing oligosaccharide” can comprise a single LNnT core sequence, a repetitive series of LNnT core sequences, or one or more LNnT core sequences within a larger sugar that further contains one or more additional mono- or disaccharides. For example, an LNnT-containing oligosaccharide can be a trisaccharide, a tetrasaccharide, a pentasaccharide, etc. which retains the immunomodulatory capacity described herein.

As used herein, the term “multivalent lacto-N-neotetraose” (multivalent LNnT) is intended to refer to a synthesized construct comprising multiple copies of LNnT-containing oligosaccharides, such as a form in which multiple LNnT-containing oligosaccharides are conjugated to a carrier molecule. In various embodiments, the carrier molecule can be a protein (e.g., HSA), a biodegradable polymer, or a polysaccharide or carbohydrate (e.g., dextran). Generally, the term “LNnTx,” as used herein, is intended to refer to a class of multivalent LNnT wherein x is an integer that refers to the degree of LNnT substitution on each carrier molecule (e.g., LNnT35, LNnT25, etc). Additionally, the carrier molecule can be designated by the full name of the carrier, or as a suffix (e.g., LNnT-HSA, LNnT-HSA, LNnT-dex or LNnT-dextran) to refer to the class of multivalent LNnT molecules containing the carrier molecule. Similarly, particular examples of multivalent are further referred to, for example, as LNnT25-HSA, LNnT25-dex.

As used herein, the term “monovalent lacto-N-neotetraose” (monovalent LNnT) is intended to refer to a LNnT-containing oligosaccharide comprising a single moiety of the oligosaccharide, such as a free, unconjugated, (i.e., non-crosslinked) form of the sugar.

As used herein, the term “agent comprising LNnT” is intended to refer to a molecule or molecules that includes the LNnT carbohydrate moiety with the proviso that the agent is not an antigen from S. mansoni, or an antigen from Toxocara canis, or is not Ascaris body fluid, or is not a filarial protein capable of inducing polyclonal IgE.

As used herein, the term “Lewis^{y} oligosaccharide” refers to a lacto type II carbohydrate comprising the structure: {Fuc(α1-2)Gal(β1-4)[Fuc(α1-3)]GlcNac}.

As used herein, the term “Lewis^{x} As used herein, refers to a lacto type II carbohydrate comprising the structure: {Gal(β1-4)[Fuc(α1-3)]GlcNac}.

As used herein, the term “human immune cell” is intended to include cells of the human immune system which are capable of producing cytokines. Examples of human immune cells include human T cells, human macrophages and human B cells.

As used herein, the term “T cell” (i.e., T lymphocyte) is intended to include all cells within the T cell lineage, including thymocytes, immature T cells, mature T cells and the like, from a mammal (e.g., human or mouse).

As used herein, a “T helper type 2 response” (Th2 response) refers to a response by CD4⁺ T cells that is characterized by the production of one or more cytokines selected from IL-4, IL-5, IL-6 and IL-10, and that is associated with efficient B cell “help” provided by the Th2 cells (e.g., enhanced IgG1 and/or IgE production). As used herein, the term “a cytokine that regulates development of a Th2 response” is intended to include cytokines that have an effect on the initiation and/or progression of a Th2 response, in particular, cytokines that promote the development of a Th2 response. Preferred cytokines that are produced by the methods of the invention are IL-4, IL-5 and IL-10.

As used herein, a “type-1 immune response” (“Th1 response”) includes a response by CD4+ T ells that is characterized by the expression, production or secretion of one or more type-1 cytokines and that is associated with delayed type hypersensitivity responses. The phrase “type-1 cytokine” (“Th1 cytokine”) includes a cytokine that is preferentially or exclusively expressed, produced or secreted by a Th1 cell, that favors development of Th1 cells and/or that potentiates, enhances or otherwise mediates delayed type hypersensitivity reactions. Preferred Th1 cytokines include, but are not limited to, Granulocyte-macrophage colony stimulating factor (GM-CSF), IL-2, IL-12, IL-15, IL-18, interferon-γ (IFN-γ), and tumor necrosis factor-α (TNF-α) and TNF-β.

Cytokine expression, secretion or production can also be an indicator of an immune response, for example, an indicator of a type-1 or type-2 immune response. For example, a “cytokine profile” can be indicative of a type-1 or type-2 immune response. The term “cytokine profile” includes expression, production or secretion of at least one cytokine associated with a particular type of immune response (e.g., a type-1 or type-2 immune response) and/or includes diminished or reduced expression, production or secretion of at least one cytokine associated with a mutually exclusive type of immune response (e.g., a type-2 or type-1 immune response, respectively). For example, a type-1 cytokine profile can include enhanced or increased expression, production or secretion of at least one of IL-2, IL-12, IFN-γ, and TNF-β and/or can include reduced or decreased expression, production or secretion of at least one of IL-4, IL-5, IL-6 and IL-10. Likewise, a type-2 cytokine profile can include expression, production or secretion of at least one of IL-4, IL-5, and IL-10 and/or can include reduced or decreased expression, production or secretion of at least one of IL-2, IL-12, IFN-γ and TNF-β.

The phrase “type-1 immunity” includes immunity characterized predominantly by type-1 immune responses (e.g., delayed type hypersensitivity, macrophage activation and or cellular cytotoxicity), by expression, production or secretion of at least one type-1 cytokine and/or expression of a type-1 immunity cytokine profile. A “Th1-associated autoimmune disorder or disease” is characterized by a dysfunction in a type-1 immune response.

The phrase “type-2 immunity” includes immunity characterized predominantly by type-2 immune responses (e.g., B cell help, IgG₁ and/or IgE production, eosinophil activation, mast cell stimulation and/or macrophage deactivation), by expression, production or secretion of at least one type-2 cytokine and/or expression of a type-2 immunity cytokine profile.

The phrase “potentiating or potentiation of a type-1 or type-2 immune response” includes upregulation, stimulation or enhancement of a type-1 or type-2 response, respectively (e.g., commitment of T helper precursors to either a Th1 or Th2 lineage, further differentiation of cells to either the Th1 or Th2 phenotype and/or continued function of Th1 or Th2 cells during an ongoing immune response). For a review of Th1 and Th2 subsets see, for example, Seder and Paul (1994) Ann. Rev. Immunol. 12:635-673.

The term “treatment” or “treating” as used herein refers to either (1) the prevention of a disease (prophylaxis), or (2) the reduction or elimination of symptoms of the disease of interest (therapy).

The terms “prevention”, “prevent” or “preventing” as used herein refers to inhibiting, averting or obviating the onset or progression of a disease (prophylaxis).

As used herein, the terms “immune” and “immunity” refers to the quality or condition of being able to resist a particular disease.

The terms “immunize” and “immunization,” as used herein, refer to the act of making a subject (1) not susceptible to a disease; or (2) less responsive to a disease; or (3) have an increased degree of resistance to a disease.

As used herein, an “effective amount” of an agent comprising LNnT refers to an amount of such agent comprising LNnT which is effective, either alone or in combination with a pharmaceutically acceptable carrier, upon single- or multiple-dose administration to the subject, e.g., a patient, at inhibiting the progression or symptoms of a disease, preventing the onset of symptoms, or inducing the regression or symptoms or a disease. These effects can be ascertained using routine diagnostic techniques known in the art and may manifest as a prolongation of the survival of a subject, reduction in symptoms experienced by the subject, the cure of the subject or the prevention of onset of disease in a subject.

As used herein, the language “subject” is intended to include human and nonhuman animals. In preferred embodiments, the subject is a human patient with an autoimmune disorder. The term “mammals” of the invention includes all vertebrates, e.g., such as nonhuman primates, sheep, dog, cat, horse, and cows.

As used herein, the various forms of the term “modulation” are intended to include stimulation (e.g., increasing or upregulating a particular response or activity) and inhibition (e.g., decreasing or downregulating a particular response or activity).

As used herein, the term “contacting” (i.e., contacting an agent with a cell) is intended to include incubating the agent and the cell together in vitro (e.g., adding the agent to cells in culture) and administering the agent to a subject such that the agent and cells of the subject are contacted in vivo.

Various aspects of the invention are described in further detail in the following subsections.

I. Immunomodulatory Agents

In the immunomodulatory methods of the invention, a cell (e.g., a human immune cell, macrophage or T cell) is contacted either in vitro or in vivo with an agent such that an immune response is modulated. Preferably, the agent itself comprises LNnT, as described in further detail below. In one embodiment, the agent stimulates production by the cell of at least one cytokine (e.g., a cytokine that regulates development of a Th2 response). In another embodiment, the agent stimulates production of IL-4. In another embodiment, the agent stimulates cellular proliferation (e.g., B cell proliferation). In yet another embodiment, the agent stimulates production of non-specific polyclonal IgE. In yet another embodiment, the agent inhibits Th1 cell dysfunction, e.g., associated with an autoimmune disorder.

A. Stimulatory Agents

The agents of the invention stimulate cytokine production by cells, stimulate production of non-specific polyclonal IgE by cells, and/or stimulate proliferation of cells. Accordingly, in one embodiment, the agent is a stimulatory form of a compound comprising LNnT. A “stimulatory form of a compound comprising LNnT” is one in which the carbohydrate structure (e.g., LNnT, or a LNnT-containing oligosaccharide) is present in a multivalent, crosslinked form.

In a preferred embodiment, the stimulatory form of a compound comprising LNnT is a conjugate of a carrier molecule and multiple carbohydrate molecules (e.g., the LNnT, or a LNnT-containing oligosaccharide). For example, carbohydrate molecules can be conjugated to a protein carrier, such as a conjugate of human serum albumin (HSA). When a sugar-carrier protein conjugate is to be administered to a subject, the carrier protein should be selected such that an immunological reaction to the carrier protein is not stimulated in the subject (e.g., a human carrier protein should be used with a human subject, etc). Alternative to a carrier protein, multivalent LNnT can be conjugated to other carrier molecules, for example carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Other preferred carriers include polymers, such as carbohydrate or polysaccharide polymers. A preferred carbohydrate polymer is dextran. Typically, carbohydrates or polysaccharides that are useful as a carrier molecule have a molecular weight of about 5,000 to 100,000 daltons, preferably between about 8,000 to 80,000 daltons, more preferably about 10,000 to 50,000, or 10,000 to 40,000 daltons.

The degree of stimulatory ability of the conjugate is influenced by the density of sugars conjugated to the carrier. Preferably, the sugar molecules comprise at least 10% of the conjugate by weight, more preferably at least 15% of the conjugate by weight, even more preferably at least 20% of the conjugate by weight and even more preferably at least 25% of the conjugate by weight or at least 30% of the conjugate by weight or at least 35% of the conjugate by weight or at least 40% of the conjugate by weight or at least 45% of the conjugate by weight. In certain embodiments, the sugar molecules comprise about 10-25% of the conjugate by weight, about 15-25% of the conjugate by weight or about 20-25% of the conjugate by weight or about 30-35% by weight or about 35-40% by weight or about 40-45% by weight. In a preferred embodiment, the stimulatory form of a compound comprising LNnT is a conjugate of multiple carbohydrate molecules. More preferably, the conjugates comprise 10-11, 12-13, 14-15, 6-17, 18-19, or 20 or more sugars/conjugate. Even more preferably, the stimulatory form of multivalent LNnT is a conjugate containing 25, 30, 25, 40, 45, 50 or more sugars/conjugate.

Agents for use in the methods of the invention can be purchased commercially For example, multivalent conjugates of LNnT and a carrier protein (e.g., HSA), carbohydrate molecule (e.g., dextran), or other polymer (e.g., polyacrylamide) are available from Accurate Chemical and Scientific Corporation (Westbury, N.Y.), Glycotech (Rockville, Md.),Neose Technologies (Horsham, Pa.), CarboMer, Inc., and Dextran Laboratories.

Alternatively, multivalent forms of LNnT can be generated using standard methods. For example, the oligosaccharide portion of the LNnT is bound to a multivalent carrier using techniques known in the art so as to produce a conjugate in which more than one individual molecule of the oligosaccharide is covalently attached to the multivalent carrier. The multivalent carrier is sufficiently large to provide a multivalent molecule leaving from between 2-1,000 (i.e. p=an integer of 2-1,000), preferably 2-100, more preferably 2-50, and more preferably 10-50 molecules of the oligosacharride portion bound to the multivalent carrier. In certain preferred embodiments 13, 25, 35, 45, 50, 100 or 200 LNnT-containing molecules are bound to the multivalent.

Suitable multivalent carriers include compounds with multiple binding sites capable of forming a bond with a terminal linking group which is capable of binding to the reducing end saccharide, or with multiple binding sites capable of forming a bond to the C₁ glycosidic oxygen of a glucose or N-acetylglucosamine residue. Examples include, but are not limited to, a polyol, a polysaccharide, polylysine avidin, polyacrylamide, a carbohydrate (e.g., dextran), lipids, lipid emulsions, liposomes, a dendrimer, a protein (e.g., human serum albumin (HSA), bovine serum albumin (BSA)) or a cyclodextran

The chemistry necessary to create the multivalent molecule and to link the oligosaccharide to the multivalent carrier are well known in the field of linking chemistry. Non-limiting examples of linkage chemistry in accordance with the present invention include those described in U.S. Pat. No. 5,736,533, Stowell et al. (Stowell et al. (1980) Advances in Carbohydrate Chemistry and Biochemistry, 37:225) and Smith et al. (Smith et al. (1978) Complex Carbohydrates part C, Methods in Enzymology, volume L, Ed. by V. Ginsburg, pg. 169), each of which is hereby incorporated by reference herein

For example, a bond between the reducing end saccharide and the carrier molecule can be formed by reacting an aldehyde or carboxylic acid at C1 of the reducing end saccharide or any aldehyde or carboxylic acid group introduced onto the reducing end saccharide by oxidation, with the carrier molecule to form a suitable bond such as —NH—, —N(R′)_ where R′ is C1-20 alkyl, a hydroxyalkylamine, an amide, an ester, a thioester, a thioamide. The bond between the reducing end saccharide and the carrier molecule can also be formed by reacting the C1 hydroxyl group, in the pyranose form, with an acylating agent and a molecular halide, followed by reaction with a nucleophile to form a suitable bond such as —NH—, —N(R′)— where R′ is C1-20 alkyul (as described by Stowell et al., supra). The oligosaccharide portion can be bound to the multivalent carrier via the free anomeric carbon of the reducing end saccharide. Alternatively, the reducing end saccharide can be bound via a phenethylamine-isothyocyanate derivative as described by Smith et al., supra. A wide variety of other bifunctional and polyfunctional cross-linking agents that can be used to form multivalent conjugates are known in the art and readily available (e.g., Pierce Chemical Co., Rockford, Ill.). In addition to conjugates comprising LNnT described above, another form of a stimulatory agent comprising LNnT is an isolated protein that naturally expresses LNnT in a form suitable for stimulatory activity.

In certain embodiments of the invention, the LNnT-containing oligosaccharide is conjugated to a protein carrier, preferably HSA. Particular examples of multivalent LNnT molecules include, but are not limited to, LNnT13, LNnT21, LNnT25, LNnT35, LNnT45 and LNnT50 conjugated to HSA or dextran.

The ability of an agent of the invention to stimulate production by immune cells of a cytokine can be evaluated using an in vitro culture system such as that described in the Examples. Cells are cultured in the presence of the agent to be evaluated in a medium suitable for culture of the chosen cells. After a period of time (e.g., 24, 48, 72, or 120 hours), production of the cytokine is assessed by determining the level of the cytokine in the culture supernatant. Preferably, the cytokine assayed is IL-4. Additionally or alternatively, IL-2, IL-5, IL-10, IL-13 and/or IFN-γ levels can be assessed. Cytokine levels in the culture supernatant can be measured by standard methods, such as by an enzyme linked immunosorbent assay (ELISA) utilizing a monoclonal antibody that specifically binds the cytokine. The ability of the agent to stimulate cytokine production is evidenced by a higher level of cytokine (e.g., IL-4) in the supernatants of cells cultured in the presence of the agent compared to the level of cytokine in the supernatant of cells cultured on the absence of the agent.

The ability of an agent of the invention to stimulate production of non-specific polyclonal IgE by cells (e.g., immune cells) can be evaluated in vitro utilizing methods such as those described in the Examples. For example, serum isolated from a subject can be analyzed by sandwich ELISA for the presence of total, as well as antigen-specific, IgE. Briefly, plates are coated with anti-IgE antibodies, washed extensively, blocked to prevent non-specific adsorption of reagents to the plate, then incubated with serum samples isolated from subjects. Labeled antibody (e.g., biotinylated anti-IgE antibody) can be used to detect antigen, for example, by detection with peroxidase-conjugated strepavidin. The reactions can be subsequently developed using, for example, tetramethyl-benzidine substrate. Such methods are further useful for detection of, for example, Ag-specific IgG, HSA-specific IgE, LNnT-HSA-specific IgE, as well as specific IgG subtypes, by altering the specificity of the primary antibody (e.g., that used in initial coating of the plate).

The ability of an agent of the invention to stimulate proliferation of cells (e.g., proliferation responses) can be evaluated in vitro utilizing methods such as those described in the Examples. For example, spleen cells can be isolated from sacrificed mice, cultured in vitro in appropriate culture medium, and labeled with ³H thymidine as an indicator of DNA replication.

B. Inhibitory Agents

The inhibitory agents of the invention can inhibit induction of a Gr1+, CD11b+ suppressor cell population that is recruited by multivalent LNnT treatment. Accordingly, in one embodiment, the agent is an inhibitory form of a compound comprising LNnT. An “inhibitory form of a compound comprising LNnT” is one in which the carbohydrate structure (e.g., the LNnT, or a LNnT-containing oligosaccharide) is present in a monovalent, non-crosslinked form. LNnT is commercially available (e.g., as a custom order from GlycoTech, Rockville Md.). LNnT-containing oligosaccharides can be generated using standard methods for linking saccharide molecules, such as those described herein (e.g., Stowell et al., supra).

LNnT can also be isolated using methods well-known to those skilled in the art from pooled human milk or produce by chemical synthesis. For example, LNnT can be synthesized chemically by enzymatic transfer of saccharide units from donor moieties to acceptor moieties using glycosyltransferases as described in U.S. Pat. Nos. 5,288,637 and 5,945,314, and WO 96/10086, each of which is hereby incorporated by reference herein.

II. Pharmaceutical Compositions

Another aspect of the invention pertains to pharmaceutical compositions of the agents (e.g., stimulatory agents) of the invention. The pharmaceutical compositions of the invention typically comprise an agent of the invention and a pharmaceutically acceptable carrier. As used herein “pharmaceutically acceptable carrier” includes any and all solvents, dispersion media, coatings, antibacterial and antifungal agents, isotonic and absorption delaying agents, and the like that are physiologically compatible. The type of carrier can be selected based upon the intended route of administration. In various embodiments, the carrier is suitable for intravenous, intraperitoneal, subcutaneous, intramuscular, transdermal or oral administration. In a preferred embodiment, the composition is formulated such that it is suitable for intraperitoneal administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, monostearate salts and gelatin. Moreover, the modulators can be administered in a time release formulation, for example in a composition which includes a slow release polymer. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Depending on the route of administration, the agent may be coated in a material to protect it from the action of enzymes, acids and other natural conditions which may inactivate the agent. For example, the agent can be administered to a subject in an appropriate carrier or diluent co-administered with enzyme inhibitors or in an appropriate carrier such as liposomes. Pharmaceutically acceptable diluents include saline and aqueous buffer solutions. Enzyme inhibitors include pancreatic trypsin inhibitor, diisopropylfluorophosphate (DEP) and trasylol. Liposomes include water-in-oil-in-water emulsions as well as conventional liposomes (Strejan, et al., (1984) J. Neuroimmunol 7:27). Dispersions can also be prepared in glycerol, liquid polyethylene glycols, and mixtures thereof and in oils. Under ordinary conditions of storage and use, these preparations may contain a preservative to prevent the growth of microorganisms.

The active agent in the composition (i.e., a stimulatory or inhibitory agent of the invention) preferably is formulated in the composition in a therapeutically effective amount. A “therapeutically effective amount” refers to an amount effective, at dosages and for periods of time necessary, to achieve the desired therapeutic result, such as the production of sufficient levels of non-specific polyclonal IgE, or the amelioration or prevention of Th1-associated autoimmune disease, to thereby influence the therapeutic course of a particular disease state. A therapeutically effective amount of an active agent may vary according to factors such as the disease state, age, sex, and weight of the individual, and the ability of the agent to elicit a desired response in the individual. Dosage regimens may be adjusted to provide the optimum therapeutic response. A therapeutically effective amount is also one in which any toxic or detrimental effects of the agent are outweighed by the therapeutically beneficial effects. In another embodiment, the active agent is formulated in the composition in a prophylactically effective amount. A “prophylactically effective amount” refers to an amount effective, at dosages and for periods of time necessary, to achieve the desired prophylactic result, for example, influencing the production of sufficient levels of non-specific polyclonal IgE for prophylactic purposes. Typically, since a prophylactic dose is used in subjects prior to or at an earlier stage of disease, the prophylactically effective amount will be less than the therapeutically effective amount.

A non-limiting range for a therapeutically or prophylactically effective amounts of a stimulatory or inhibitory agent of the invention is 0.01 nM-20 mM. Alternatively, a stimulatory or inhibitory agent can be used in an amount between 500 μg to 100 mgs. It is to be noted that dosage values may vary with the severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The amount of active compound in the composition may vary according to factors such as the disease state, age, sex, and weight of the individual. Dosage regimens may be adjusted to provide the optimum therapeutic response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

An agent of the invention can be formulated into a pharmaceutical composition wherein the agent is the only active compound therein. Alternatively, the pharmaceutical composition can contain additional active compounds. For example, two or more agents may be used in combination. Moreover, an agent of the invention can be combined with one or more other agents that have immunomodulatory properties. For example, a stimulatory agent may be combined with one or more cytokines or adjuvants.

A pharmaceutical composition of the invention, comprising a stimulatory or inhibitory agent of the invention, can be administered to a subject to modulate immune responses (e.g., production of non-specific polyclonal IgE, inhibit Th1 responses associated with autoimmune disorder) in the subject. As used herein, the term “subject” is intended to include living organisms in which an immune response can be elicited, e.g., mammals. Examples of subjects include humans, dogs, cats, mice, rats, and transgenic species thereof.

A pharmaceutical composition of the invention can be formulated to be suitable for a particular route of administration. For example, in various embodiments, a pharmaceutical composition of the invention can be suitable for injection, inhalation or insufflation (either through the mouth or the nose), or for intranasal, mucosal, oral, buccal, parenteral, rectal, intramuscular, intravenous, intraperitoneal, and subcutaneous delivery.

A pharmaceutical composition of the invention, comprising multivalent LNnT can be administered to a subject to immunize a subject against a Th1 associated autoimmune disease. The vaccines are administered in a manner compatible with the dosage formulation, and in such amount as will be therapeutically effective and immunogenic. For example, vaccines containing the multivalent conjugates of the invention can be administered intradermally, transdermally, orally, intranasally, intramuscularly, subcutaneously, intravenously or intraperitoneally by a variety of methods known to those skilled in the art, including but not limited to needle and syringe administration, microseeding, transdermal patch, inhalation, spray, tablet, liquid drink, and biolistics. The quantity to be administered depends on the subject to be treated, capacity of the subject's immune system to generate a cellular immune response, and degree of protection desired. The multivalent conjugates can be used in combination with a variety of immunogens including but not limited to subunit vaccines, proteins, peptides, carbohydrates, lipids and nucleic acids, as well as whole live or attenuated organisms or altered pathogens. Precise amounts of active ingredient required to be administered depend on the judgment of the practitioner and are peculiar to each individual. However, suitable dosage ranges are of the order of about one microgram to about one milligram, preferably about 500 to 800 micrograms, and preferably about 1 microgram and more preferably about 5 micrograms, and more preferably 100 micrograms active ingredient per kilogram bodyweight individual. Suitable regimes for initial administration and booster shots are also variable, but are typified by an initial administration followed by a subsequent administration. In some cases, a single dose may be sufficient to induce immunity and/or alleviate symptoms in a subject. In other cases a typical prime boost regimen is implemented, with the precise number of boosts needed for any single vaccine containing the multivalent conjugates to be determined by measuring the desired immunological outcomes relevant to the specific vaccine (e.g., antibody titer, delayed type hypersensitivity response, proliferation of B and/or T cells, cytokine and/or chemokine responses). Examples of method for assaying for immune response to vaccines are readily available, e.g., Immunobiology. 5th ed., Appendix I: A36-A41, Janeway, Charles A.; Travers, Paul; Walport, Mark; Shlomchik, Mark. New York and London: Garland Publishing; c2001.

In certain embodiments, a pharmaceutical composition of the invention can be packaged with instructions for using the pharmaceutical composition for a particular purpose, such as to modulate an immune response, for use as an adjuvant, to modulate an allergic response or to modulate an autoimmune disease.

III. Modulation of Immune Responses

The invention provides immunomodulatory methods that can be used to modulate various immune responses. In the methods of the invention, a cell is contacted with an agent (e.g., an agent comprising multivalent LNnT) with the cell such that the immune response is modulated (e.g., stimulated). The methods of the invention can be practiced either in vitro or in vivo. For practicing the method of the invention in vitro, cells can be obtained from a subject by standard methods and incubated (i.e., cultured) in vitro with an agent of the invention to modulate, for example, the production of a cytokine (e.g., IL-4), the production of non-specific, polyclonal IgE, proliferation of an immune cell (e.g., a splenocyte), the development of a Th2 response, and/or the inhibition of a Th1 response. For example, peripheral blood mononuclear cells (PBMCs) can be obtained from a subject and isolated by density gradient centrifugation, e.g., with Ficoll/Hypaque. Specific cell populations can be depleted or enriched using standard methods. For example, monocytes/macrophages can be isolated by adherence on plastic. T cells or B cells can be enriched or depleted, for example, by positive and/or negative selection using antibodies to T cell or B cell surface markers, for example by incubating cells with a specific mouse monoclonal antibody (mAb), followed by isolation of cells that bind the mAb using anti-mouse-Ig coated magnetic beads. Monoclonal antibodies to cell surface markers are commercially available.

For practicing the methods of the invention in vivo, an agent is administered to a subject in a pharmacologically acceptable carrier (as described in the previous section) in amounts sufficient to achieve the desired effect, such as to modulate, for example, the production of a cytokine, the production of non-specific, polyclonal IgE, proliferation of an immune cell, the inhibition of a Th1 response, and/or the development of a Th2 response in the subject or to prevent a detrimental host reaction against parasite infection, to protect against environmental allergens by saturating FcεRs on effector cells in the subject or to inhibit a disease or disorder (e.g., an allergy or an Th1-associated autoimmune disease) in the subject. Any route of administration suitable for achieving the desired immunomodulatory effect is contemplated by the invention. One preferred route of administration for the agent is intraperitoneal. Another preferred route of administration is orally. Yet another preferred route of administration is intravenous. Application of the methods of the invention to the treatment of disease conditions may result in cure of the condition, a decrease in the type or number of symptoms associated with the condition, either in the long term or short term (i.e., amelioration of the condition) or simply a transient beneficial effect to the subject.

Numerous disease conditions associated with a predominant Th2-type response have been identified and could benefit from modulation of the type of response mounted in the individual suffering from the disease condition. Application of the immunomodulatory methods of the invention to such diseases as cancer, infectious disease, allergies, autoimmune disease, and inflammatory bowel disease. In addition to the foregoing disease situations, the immunomodulatory methods of the invention also are useful for other purposes. For example, the methods of the invention (i.e., methods using a stimulatory agent) can be used to stimulate production cytokines (such as IL-4) in vitro for commercial production of these cytokines (e.g., cells can be cultured with a stimulatory agent in vitro to stimulate IL-4 production and the IL-4 can be recovered from the culture supernatant, further purified if necessary, and packaged for commercial use).

Another aspect of the invention pertains to use of a stimulatory agent of the invention in the treatment or prevention of shock in a subject. As demonstrated in Example 3, administration of multivalent LNnT (e.g., LNnT conjugated to dextran) renders spleen cells less responsive to LPS, as measured by proliferative responses and production of type 1 cytokines such as IL-12 and IFN-γ. The production of IL-12 and IFN-γ are associated with the onset of inflammatory responses, including shock (e.g., see Oberholzer et al., Crit. Care Med. 28(4Suppl):N3-12, 2000). Thus, multivalent LNnT treatment can be used to inhibit the shock response in a patient. For at risk patients, these patients can be pretreated with a multivalent LNnT composition of the invention to render them less susceptible to shock. Accordingly, the invention provides a method for inhibiting or preventing shock in a subject comprising administering an agent comprising multivalent lacto-N-neotetraose (LNnT), such that shock is inhibited or prevented in the subject. The subject may be a patient already experiencing at least one symptom of shock or, more preferably, is a patient at risk of (susceptible to) shock, such as a surgery patient who is susceptible to shock. For at risk patients, the multivalent LNnT agent can be administered to the patient at least 1 hour prior to a time when shock may develop in the patient or at least 2 hours, 3 hours, 6 hours, 12 hours, 24 hours, 48 hours, 72 hours or more prior to a time when shock may develop in the patient.

Yet another aspect of the invention pertains to a method of inhibiting induction of Gr1+, CD11b+ suppressor cells in a subject. This method can be used to inhibit induction of the suppressor cells in a clinical setting where such inhibition is desirable, such as in the treatment of cancer. The method comprises: administering to the subject an agent comprising monovalent lacto-N-neotetraose (LNnT), such that induction of Gr1+, CD11b+ suppressor cells in the subject is inhibited. As demonstrated in Example 4, administration of multivalent LNnT induces this suppressor cell population, which is capable of inhibiting T cell proliferative responses. This suppressor cell population may be induced in a clinical setting in a patient by tumor cells that express multivalent LNnT, and the induction of such a suppressor population could promote tumor growth and expansion, by suppression of immune responses against the tumor and/or by production by the suppressor population of factors (such as TGF-β) that promote tumor angiogenesis. Accordingly, inhibition of the induction of this suppressor population can be used in the treatment of cancer, by administration of an agent comprising monovalent LNnT to thereby competitively block the induction of the suppressor population. In one embodiment, the agent is administered intraperitoneally. In another embodiment, the agent is administered intravenously. Preferably, the method for inhibiting induction of the suppressor population is carried out in a subject suffering from cancer.

The stimulatory methods of the invention (i.e., methods using the stimulatory agents of the invention) also can be used therapeutically for treating type 1 autoimmune diseases (i.e., autoimmune diseases that are associated with Th1-type dysfunction). Many autoimmune disorders are the result of inappropriate activation of T cells that are reactive against self tissue and that promote the production of cytokines and autoantibodies involved in the pathology of the diseases. It has been shown that modulation of T helper-type responses can either have a beneficial or detrimental effect on an autoimmune disease. For example, in experimental allergic encephalomyelitis (EAE), stimulation of a Th2-type response by administration of IL-4 at the time of the induction of the disease diminishes the intensity of the autoimmune disease (Paul, W. E., et al. (1994) Cell 76:241-251). Furthermore, recovery of the animals from the disease has been shown to be associated with an increase in a Th2-type response as evidenced by an increase of Th2-specific cytokines (Koury, S. J., et al. (1992) J. Exp. Med. 176:1355-1364). Moreover, T cells that can suppress EAE secrete Th2-specific cytokines (Chen, C., et al. (1994) Immunity 1:147-154). Since stimulation of a Th2-type response in EAE has a protective effect against the disease, stimulation of a Th2 response (and/or downmodulation of a Th1 response) in subjects with multiple sclerosis (for which EAE is a model) may be beneficial therapeutically.

Similarly, stimulation of a Th2-type response in type 1 diabetes in mice provides a protective effect against the disease. Indeed, treatment of NOD mice with IL-4 (which promotes a Th2 response) prevents or delays onset of type 1 diabetes that normally develops in these mice (Rapoport, M. J., et al. (1993) J. Exp. Med. 178:87-99). Thus, stimulation of a Th2 response (and/or downmodulation of a Th1 response) in a subject suffering from or susceptible to diabetes may ameliorate the effects of the disease or inhibit the onset of the disease.

Yet another autoimmune disease in which stimulation of a Th2-type response may be beneficial is rheumatoid arthritis (RA). Studies have shown that patients with rheumatoid arthritis have predominantly Th1 cells in synovial tissue (Simon, A. K., et al., (1994) Proc. Natl. Acad. Sci. USA 91:8562-8566). By stimulating a Th2 response in a subject with RA, the detrimental Th1 response can be concomitantly downmodulated to thereby ameliorate the effects of the disease.

To treat a type 1 autoimmune, a stimulatory agent of the invention (e.g., an agent comprising multivalent LNnT) can be administered to the subject, for a variety of therapeutically beneficial purposes, including downmodulating the production of the Th1-associated cytokines IL-12 and IFN-γ, and induction of Gr1+, CD11b+ suppressor cells. The stimulatory agent can be used alone, or in combination with one or more additional agents that promote Th2 responses (e.g., Th2-promoting cytokines, such as IL-4 or IL-10) and/or downmodulate Th1 responses (e.g., antibodies to Th1-promoting cytokines such as anti-IL-2, anti-IL-12, anti-IFN-γ). Depending on the disease, the stimulatory agent may be administered either systemically or locally. For example in the case of rheumatoid arthritis, the agent may be administered directly into the joints. For systemic treatment, the stimulatory agent preferably is administered intravenously. Alternative to direct administration of the stimulatory agent to the subject, autoimmune diseases may be treated by an ex vivo approach. In this case, cells (e.g., T cells, macrophages, B cells, peritoneal exudate cells) are obtained from a subject having an autoimmune disease, cultured in vitro with a stimulatory agent of the invention, for example, to stimulate generation of the Gr1+, CD11b+ suppressor cell population and/or to inhibit production of Th1-associated cytokines (e.g., IL-12, IFN-γ) and/or to stimulate production of Th2-associated cytokines (e.g., IL-13), followed by readministration of the cells to the subject.

Non-limiting examples of type-1 autoimmune diseases and disorders having an type-1 autoimmune component that may be treated according to the invention include diabetes mellitus, inflammatory bowel disease, arthritis (including rheumatoid arthritis, juvenile rheumatoid arthritis, osteoarthritis, psoriatic arthritis), multiple sclerosis, myasthenia gravis, autoimmune thyroiditis, dermatitis (including atopic dermatitis and eczematous dermatitis), psoriasis, Sjögren's Syndrome, including keratoconjunctivitis sicca secondary to Sjögren's Syndrome, alopecia areata, allergic responses due to arthropod bite reactions, Crohn's disease, aphthous ulcer, iritis, conjunctivitis, keratoconjunctivitis, ulcerative colitis, scleroderma, vaginitis, proctitis, drug eruptions, leprosy reversal reactions, erythema nodosum leprosum, autoimmune uveitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves ophthalmopathy, sarcoidosis, primary biliary cirrhosis, uveitis posterior, and interstitial lung fibrosis.

In a related aspect, the invention provides a method for preventing or treating a disease or a disorder in a subject prophylactically or therapeutically. Administration of an agent prophylactically (i.e. the agent of the present invention) can occur prior to the manifestation of symptoms of an undesired disease or disorder, such that the disease or disorder is prevented or, alternatively, delayed in its progression. The prophylactic methods of the present invention can be carried out in a similar manner to therapeutic methods described herein, although dosage and treatment regimes may differ.

For example, subjects at risk of developing type I diabetes can be identified and treated prophylactically with the agent of the present invention to prevent the onset of the disease. Susceptibility of a subject to type I diabetes mellitus has been associated with genes encoding the class II antigens of the major histocompatibility region of chromosome 6 (HLA-D). For instance, approximately 80% of patients with type I diabetes have either HLA-DR3 or HLA-DR4 alleles, whereas the general population the prevalence of these antigens is only 30 to 50%. Individuals who are HLA-DR3 positive have a fivefold greater risk of developing type I diabetes, and those with HLA-DR4, a seven times increased risk, compared to those who are HLA-DR3 or −4 negative. HLA-DR3/4 heterozygotes have an approximately 14.3 increased risk (see generally, Cotran, R., Kumar, V. and Robbins, S. Robbins (1989) Pathologic Basis of Disease, 4^{th} Ed. W. B. Saunders Co., Philadelphia, pgs 994-1005). Moreover, further association is linked with certain alleles in the DQ locus, which are in “linkage disequilibrium” with HLA-DR. DNA analysis from the DQβ locus of type I diabetics versus nondiabetic subjects show that simple differences in the structure of the DQ molecule, i.e. a mutation of the amino acid Asparagine57 to either Serine, Alanine or Valine, confers increased susceptibility to type I diabetes in Caucasians (Id.).

Similarly, genetic susceptibility genes located within the HLA gene complex have also been linked to psoriasis and inflammatory bowel disease. For example, the genes HLA-C, corneodesmosin and HCR have been linked to increased incidences of psoriasis (see e.g., Barker, J. (2001) Clin. Exp. Dermatol. 26:321 and Bowcock, A. et al. (2001) Hum. Mol. Genet. 10:1793). Consistent replication of linkage among patients suffering from inflammatory bowel disease (IBD) has also been found with distinct regions on chromosomes 12, 6 (major histocompatibility complex) and 14 (see e.g. Satsangi, J. (2001) Acta. Odontol. Scand. 59:187). Thus, using routine techniques known in the art (i.e. DNA sequencing, RFLP analysis, genetic linkage studies), subjects can be identified who are at risk of developing type-1 diabetes mellitus, psoriasis, and IBD and can thus be prophylactically treated with the agent of the present invention.

Another aspect of the invention pertains to methods for treating a subject therapeutically. In one embodiment, the present invention includes methods of inhibiting disease progression, e.g. downregulating a Th1-mediated immune response, in a subject. A preferred embodiment of the invention involves administering to a subject an agent that comprises LNnT, in an amount effective to inhibit disease progression in the subject. Accordingly, the present method has therapeutic utility in biasing the immune system towards inducing a type-2 immune response depending upon the desired therapeutic regimen. Such modulatory methods are particularly useful in Th1-biased autoimmune diseases.

The effectiveness of the therapeutic administration of the agent of the present invention will greatly depend on when, during the progression of the disease, treatment is started. For example, type I diabetes mellitus is characterized by the development of autoimmunity to islet beta cells. Treatment will need to have begun prior to the destruction of all of the islet beta cells. Preferably, treatment will begin as soon as the onset of the disease or at the time the disease is first diagnosed so as to prevent the destruction of more islet beta cells.

In another aspect of the invention, the agent is administered peritoneally, intravenously or subcutaneously. To treat an autoimmune disease in which a Th2-type response is beneficial to the course of the disease in the subject, an agent of the invention can be administered to the subject in amounts sufficient to stimulate a Th2-type response. The agent can be used alone, or in combination with one or more additional agents that promote Th2 responses (e.g., Th2-promoting cytokines, such as IL-4 or IL-10). Depending on the disease, the agent may be administered either systemically or locally.

Alternative to direct administration of the agent to the subject, diseases may be treated by an ex vivo approach. In this case, immune cells (e.g., T cells, macrophages and/or B cells) are obtained from a subject having an autoimmune disease, cultured in vitro with an agent of the invention to stimulate production by the cells of one or more cytokines that promote a Th2 response (e.g., IL-10), followed by readministration of the cells to the subject.

The efficacy of agents for treating autoimmune diseases can be tested in the models of human diseases (e.g., EAE as a model of multiple sclerosis and the NOD mice as a model for diabetes) or other well characterized animal models of human autoimmune diseases. Such animal models include the mrl/lpr/lpr mouse as a model for lupus erythematosus, murine collagen-induced arthritis as a model for rheumatoid arthritis, and murine experimental myasthenia gravis (see Paul ed., Fundamental Immunology, Raven Press, New York, 1989, pp. 840-856). An agent of the invention is administered to test animals and the course of the disease in the test animals is then monitored by the standard methods for the particular model being used. Effectiveness of the modulatory agent is evidenced by amelioration of the disease condition in animals treated with the agent as compared to untreated animals (or animals treated with a control agent).

Another aspect of the invention relates to a vaccine for immunizing a subject against a Th1 autoimmune disease, comprising an immunogenic amount of the multivalent conjugate of the present invention, either alone or dispersed in a physiologically acceptable, nontoxic vehicle, which amount is effective to immunize a subject, preferably a human, against the disease. Accordingly, the vaccines are administered in a manner compatible with the dosage formulation, and in such amount as will be therapeutically effective and immunogenic. The quantity to be administered depends on the subject to be treated, capacity of the subject's immune system to generate a cellular immune response, and degree of protection desired. Precise amounts of active ingredient required to be administered depend on the judgment of the practitioner and are peculiar to each individual. However, suitable dosage ranges are of the order of about one microgram to about one milligram, and preferably about 500 to about 800 micrograms. Preferably about 1 microgram, more preferably about 5 micrograms, and more preferably 100 micrograms active ingredient per kilogram bodyweight individual is used. Suitable regimes for initial administration and booster administrations are also variable, but are typified by an initial administration followed, if necessary by subsequent administration, for example, at weekly, bi-weekly, monthly, or yearly intervals. In some cases, a single dose may be sufficient to induce immunity and/or alleviate symptoms in a subject. In other cases, a series of immunizations may be required in order to generate the desired level of immunity.

The invention also provides pharmaceutical compositions for carrying out the methods of the invention. For example, in one embodiment, the invention provides a pharmaceutical composition comprising an agent comprising multivalent lacto-N-neotetraose (LNnT) and a pharmaceutical carrier, packaged with instructions for use of the pharmaceutical composition as a modulator of IgE responses, or for the treatment of shock or TH1-type autoimmune diseases in a subject. For these compositions, the agent can comprise LNnT-containing oligosaccharides conjugated to a protein carrier, such as human serum albumin, or LNnT-containing oligosaccharides conjugated to a carbohydrate polymer, such as dextran.

In yet another embodiment, the invention provides a pharmaceutical composition comprising an agent comprising monovalent lacto-N-neotetraose (LNnT) and a pharmaceutical carrier, packaged with instructions for use of the pharmaceutical composition for the treatment of cancer in a subject.

This invention is further illustrated by the following examples which should not be construed as limiting. The contents of all references, patents and published patent applications cited throughout this application are hereby incorporated by reference.

### EXAMPLES

Materials and Methods used in the Examples

Animals

Young adult (7-9 weeks old) CBA/J, BALB/C, and C57BL/6 strain female mice were purchased from Harlan (Indianapolis, Ind.). Female CBA/CaJ xid and age-matched control female CBA/CaJ mice were purchased from The Jackson Laboratory (Bar Harbor, Me.). IL-4 deficient BALB/C mice were generated as described (Kopf et al., J. Exp. Med. 184(3):1127-36, 1996). This deficient mice were bred and maintained at Harvard School of Public Health according to the guidelines set forth by the Harvard Medical Area Research Committee.

Antigens and Inoculations

Human serum albumin (HSA) was selected as a carrier protein for multivalent carbohydrate because HSA is a simple protein and does not contain any carbohydrate motif. Multivalent LNnT or Lewisγ were conjugated with HSA (LNnT-HSA and Le^{y}-HSA) by Accurate Chemical and Scientific Corporation (NY). In both neoglycoproteins, 13 molecules of sugar conjugated to 1 HSA molecule (e.g., LNnT13-HSA). As controls, HSA (Sigma Chemical Co., MO), HSA adsorbed to alum (Intergen Company, NY; HSA-alum), or Dulbecco's PBS (Gibco BRL, NY) were prepared for immunization. Groups of four to six mice were immunized intraperitoneally or subcutaneously with Ags (10 μg of HSA) or saline. First and second boosting immunization were performed in the same fashion 2 and 3 weeks later, respectively. Protein concentration was determined by bicinchoninic acid (BCA) assay (Pierce, Ill.).

Determination of Serum Ab Titers

Immunized mice were bled from the tail 10, 6, and 5 days following primary, first, and second boosting immunization, respectively. Total and HSA-specific IgE were determined by sandwich ELISA. In brief, ELISA plates (Corning Inc., NY) were coated overnight at 4° C. with 100 μl of 5 μg/ml rat anti-mouse IgE mAb (Biosource, Calif.) in carbonate-bicarbonate buffer, pH 9.6. After washing four times with PBS containing 0.05% Tween20 (PBS-T), plates were blocked with 200 μl PBS containing 10% FCS and 0.3% Tween20 for 2 hrs at 37° C. After washing as above, 100 μl samples of serially-diluted serum or standard mouse IgEmAb (Pharmingen, Calif.) were added in duplicate wells and incubated for 2 hrs at 37° C. Thereafter, 100 μl biotinylated anti-mouse IgEmAb (0.5 μg/ml, Pharmingen) or biotinylated HSA (1 μg/ml) were added to detect total IgE and HSA-specific IgE, respectively. After 2 hrs incubation at 37° C., plates were washed and 100 μl peroxidase-conjugated streptavidin (Sigma) diluted 1/1000 was added to each well and incubated for 1 hr at 37° C. Finally plates were washed eight times, the reactions were developed by addition of tetramethyl-benzidine substrate (Kirkegaard and Perry Laboratories, Gaithersburg, Md.) and stopped by addition of phosphoric acid (0.4M). The absorbance was measured at 450 nm in UVMax automated plate reader (Molecular Devices Corp., Menlo Park, Calif.). For biotinylation, HSA (2 mg/ml) in sodium bicarbonate buffer pH 8.5 was incubated with biotin (long arm) N-hydroxy succumide ester (Vector Lab., CA) for 2 hrs at room temperature, stopped the reaction by addition of 5 μl ethanolamine, and dialyzed overnight with PBS/0.05% sodium azide.

Ag-specific IgG ELISA were determined. Briefly, ELISA plates were coated with 100 μl Ag (2 μg/ml) overnight at 4° C. in carbonate-bicarbonate buffer, and blocked as described above. Then plates were incubated with samples from individual serum in two-fold serial dilution from 100 times for 2 hrs at 37° C., followed by goat anti-mouse IgG mAb-peroxidase conjugate (Boehringer-Mannheim, Ind.) for 1 hr at 37° C. Thereafter, plates were developed and terminated as described above. Finally the absorbance at 450 nm was measured using a UVMax automatic microplate reader. Results were expressed as endpoint titers where the endpoint was determined as the final serum dilution which yields a higher absorbance than twice of the background absorbance. Optimum dilutions of anti-mouse IgG mAb-horseradish peroxidase-labeled conjugates were determined to be 1/1000. Plasma IgE specific for LNnT-HSA was also tested for in the same fashion as this method using LNnT-HSA (2 μg/ml) as a coating Ag and biotinylated anti-mouse IgEmAb (1 μg/ml, Pharmingen) followed by avidin-peroxidase conjugate (Sigma) as a detection Ab.

Serum total IgG isotypes were also determined by ELISA. Plates were coated with 100 μl of 2 μg/ml rat anti-mouse IgG1, IgG2a, IgG2b, and IgG3 mAb (Pharmingen) overnight at 4° C. After washing and blocking described as above, 100 μl samples of serially-diluted serum or standard mouse IgG isotypes (Pharmigen) were added in duplicate wells and incubated for 2 hrs at 37° C. Thereafter, 100 μl peroxidase-conjugated polyclonal goat anti-mouse Ig (Pharmingen) diluted in 1/1000 were added and incubated for 1 hr at 37° C. After the washing, the reactions were developed and stopped as described above, and the absorbance was measured at 450 nm.

Proliferative Responses of Splenocytes

Groups of mice were killed by carbon dioxide 5 days following second boosting immunization. Spleens were removed aseptically and proliferation assays were performed as described previously (Velupillai, P. et al. (1997) J. Immunol. vol. 158 pp. 338-344). In brief, cell suspensions were prepared in RPMI 1640 supplemented with 10% FCS (Gibco), 2 mM L-glutamine, 5×10⁻⁵ M 2-mercaptoethanol, 100 Unit/ml penicillin, and 100 μg/ml streptomycin (Sigma). Red blood cells were removed by incubation in Boyle's solution (GIBCO), and 2.5×10⁶ cells per ml were added to 96-well flat-bottom tissue culture plates (Corning) in triplicate for 72 hrs at 37° C., 5% CO2 in air, with 2 μg/ml ConA (Vector), 10 μg/ml HSA or LNnT-HSA. For the final 8 hrs, cells were incubated with 1 μCi ³H thymidine (Amersham Life Science Inc., IL) and then harvested onto filter paper for scintillation counting.

Cytokine Assays

In flat-bottom 24-well culture plates (Corning), suspensions of 2.5×10⁶ cells per ml were cultured with ConA (2 μg/ml) or without restimulation at 37° C. in supplemented RPMI 1640 medium as described above. 24, 72 and 120 hrs later, the cell cultures were then centrifuged, and the culture supernatants were harvested and kept frozen (−80° C.) until assayed. Pelleted cells were resuspended then stained with mAbs coupled to FITC or PE for immunofluorescent staining. IL-2, IL-4, IL-5, IL-10 and IFN-γ levels in supernatants from ConA-stimulated or unstimulated splenocytes were measured by capture ELISA. In brief, Maxisorp microtitre plates (Nunc Laboratories Ltd., Denmark) were coated with 50 μl of capture Ab at 2.0 μg/ml (rat anti-mouse IL-5, IL-10 and IFN-γ from Pharmingen and rat anti-mouse IL-4 from Endogen, Mass.) in Carbonate-bicarbonate buffer by overnight incubation at 4° C. Wells were then washed with PBS-T four times and blocked by addition of 10% FCS in PBS (2 hours, 37° C.). 50 μl of culture supernatants and appropriate recombinant standards were then added to individual well in duplicate. For standard curves, recombinant IL-5 (0 to 5,000 pg/ml), IL-10 (0 to 25,000 pg/ml), IFN-γ (0 to 20,000 pg/ml) (Pharmingen, Calif.) and IL-4 (0 to 1,500 pg/ml) (Endogen, Mass.) were used in duplicate. Following overnight incubation at 4° C., the wells were washed and appropriate 50 μl/well biotinylated detection Ab at 1 μg/ml (rat anti-mouse IL-5, IL-10 or IFN-γ from Pharmingin and rat anti-mouse IL-4 from Endogen) were added. For the detection of bound biotinylated Ab, 50 μl of streptavidin-alkaline phosphatase conjugate (1/2000, Pharmingen) was added to each well for 45 minutes, then washed. Level of cytokine in the wells was visualized by addition of p-nitrophenyl phosphate (Sigma) in glycine buffer. Absorbance was measured at 405 nm using a UVMax automatic microplate reader.

Immunofluorescent Staining

Following in vitro culture without restimulation, splenocytes were resuspended and incubated on ice for 15 min with mAbs as follows: anti-CD45R/B220 (RA3-6B2), anti-B7-1 (16-10A 1), and anti-B7-2 (GL-1) or isotype-matched controls. All mAbs were either FITC- or PE-conjugated (Pharmingen). After washing with Hanks' balanced salt solution (Gibco) containing 0.05% sodium azide, flow cytometry analysis was performed by using a FACSCalibur flow cytometer and Cell Quest software (Becton Dickinson, Calif.). Dead cells were excluded from analysis on the basis of propidium iodide (Molecular Probes, Oreg.) staining. Lymphocytes were gated according to the physical characteristics of forward and side scatter and at least 10,000 events were acquired.

Statistics

Statistical analysis was performed using the Student's unpaired t test. A value of p<0.05 was considered significant.

### Example 1

Induction of Polyclonal IgE by Multivalent LNnT In Vivo

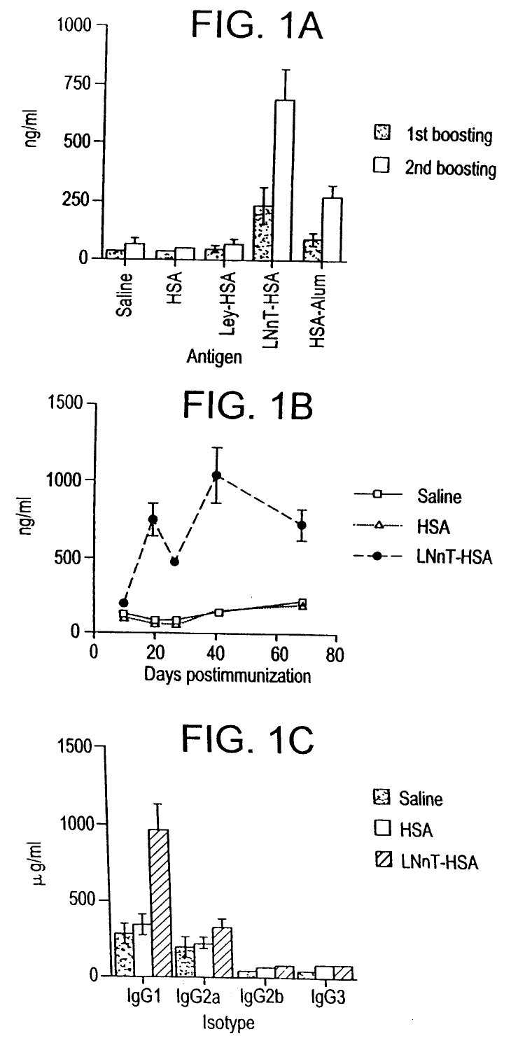

Following first boosting IP immunization with LNnT13-HSA, BALB/C mice produced significantly higher amounts of serum total IgE than those IP immunized with saline, HSA, Le^{y13}-HSA, and HSA-Alum (FIG. 1a). Serum IgE was significantly elevated following second IP immunization. This elevation was not seen following primary inoculation, however, high elevation of serum IgE lasted at least 8 weeks following second IP immunization with LNnT13-HSA (FIG. 1b). Serum IgG1 was also significantly increased in mice IP immunized with multivalent LNnT compared with those immunized with saline or HSA alone, whereas the amount of other isotypes were not significantly different (FIG. 1c).

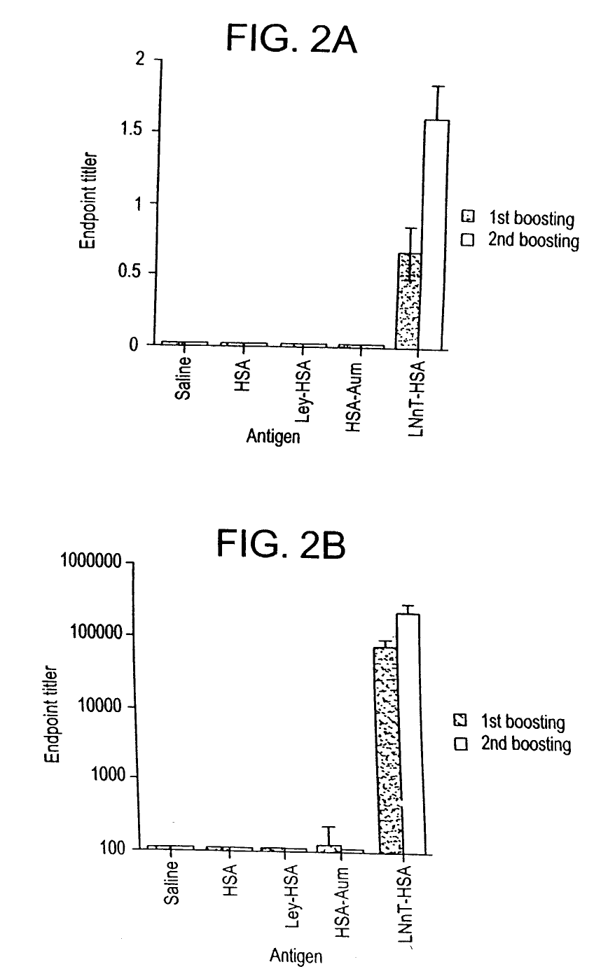

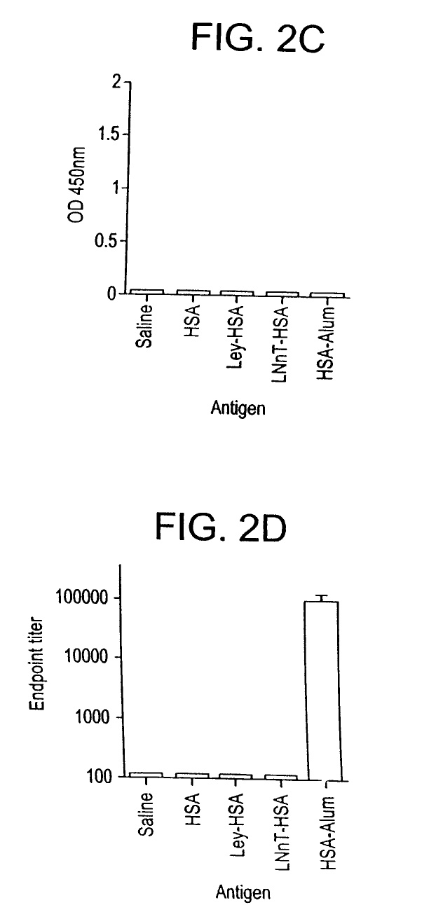

HSA-specific IgE and IgG were determined in sera in mice immunized IP with HSA-Alum. On the contrary, those signals in mice immunized IP with LNnT13-HSA were not detected and were statistically no different from the control groups immunized with HSA alone, Le^{y}-HSA, or saline (FIGS. 2a,b). Similar findings were seen in Ab titers against LNnT13-HSA. Mice IP immunized with LNnT13-HSA did not produce significant amounts of IgG or IgE specific for LNnT13-HSA compared with the controls (FIGS. 2c,d). Moreover, specific signal against Gal (β1-4) GlcNAc and Gal (β1-4) Glc, the components of LNnT, were also not detected when biotinylated Gal (β1-4) GlcNAc and Gal (β1-4) Glc (Glycotech, Md.) were used, respectively, instead of biotinylated HSA in specific IgE ELISA. These results suggested that multivalent LNnT induced nonspecific polyclonal production of IgE in BALB/C mice following boosting immunization.

Specificity of Induction of Polyclonal IgE by Multivalent LNnT

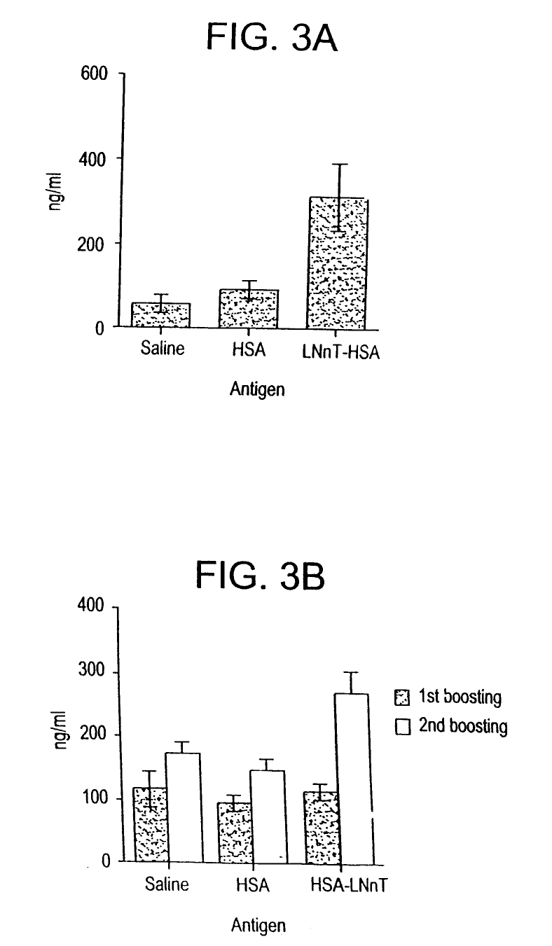

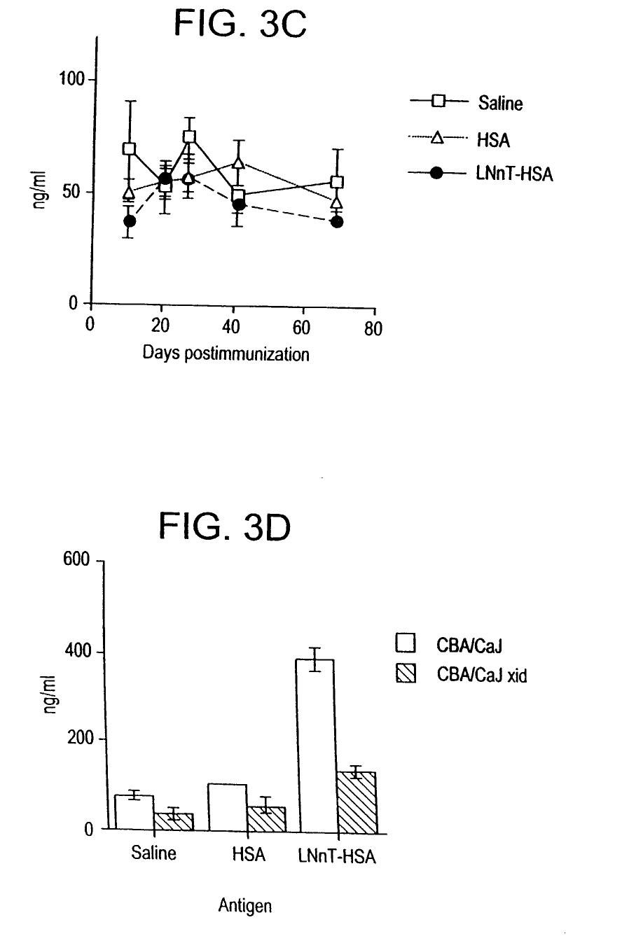

As in the BALB/C strain, CBA/J mice showed significant elevation of serum IgE following first boosting IP immunization with LNnT13-HSA when compared to the control groups (FIG. 3a). On the other hand, C57BL/6 mice did not show the increase of serum IgE following first IP immunization with LNnT13-HSA. However, this strain induced the increase of serum IgE following second IP immunization (FIG. 3b). Both strains did not elicit either IgE or IgG against HSA. These results indicate that the induction of polyclonal IgE by multivalent LNnT was genetically affected.

Elevation of serum total IgE was not seen in SC immunization with LNnT13-HSA even following the boost (FIG. 3c). It has previously been suggested that LNnT-HSA does not induce such an elevation of serum IgE following intranasal sensitization, implying that the route of inoculation of multivalent LNnT is critical for the induction of nonspecific IgE. CBA/CaJ xid mice, which are deficient CD5+ B-1 cells, showed significantly higher amounts of IgE following IP immunization with LNnT13-HSA compared with those IP immunized with saline or HSA, although the amounts were significantly lower than the control CBA/CaJ mice (FIG. 3d). This result indicates that B-1 cells, which is one of the specific components of peritoneal cavity, seems to be in part involved but not critical for the induction of polyclonal IgE by multivalent LNnT.

Proliferative Responses Against Multivalent LNnT

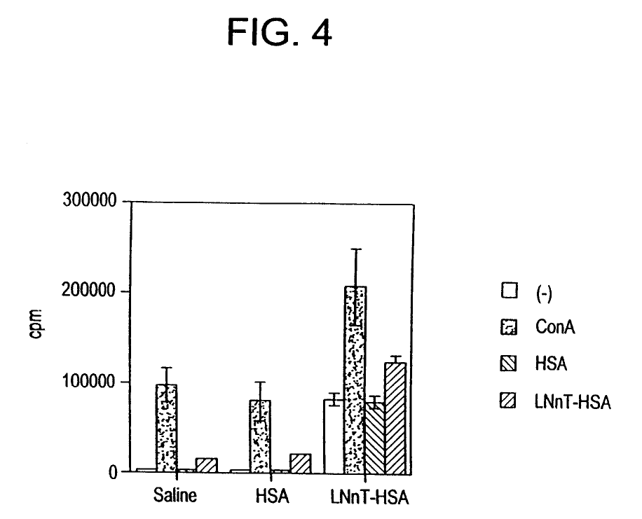

Splenocytes of BALB/C mice were prepared 5 days following second boosting IP immunization with saline, HSA or LNnT13-HSA. Mice IP immunized with HSA or saline showed moderate but significant in vitro proliferative responses to LNnT13-HSA compared to HSA or unrestimulation, suggesting that LNnT induces the proliferative responses in naive splenocytes. Mice immunized IP with LNnT13-HSA showed the significant responses even without restimulation compared to the control mice (FIG. 4). In addition, the response was significantly enhanced with restimulation of LNnT13-HSA In ConA stimulation, LNnT13-inoculated mice also responded significantly more than the controls. These results suggested that splenocytes specific for LNnT-HSA were spontaneously activated following IP immunization with multivalent LNnT.

Cytokine Production by LNnT-Inoculated Mice

Splenocytes of mice IP immunized with LNnT13-HSA also produced cytokines without restimulation. Significant amounts of IL-2, IL-4, and IL-5 were detected as early as 24 hours incubation in culture supernatants without restimulation. IL-10 and IFN-γ were detected as early as 72 hrs incubation. The results are summarized below in Table 1. Interestingly, in response to ConA, splenocytes of mice immunized IP with LNnT13-HSA produced significantly more IL4, IL-5, and IL-10, but not IFN-γ compared to those IP immunized with saline or HSA, suggesting that LNnT skewed splenocytes into polarized Th2 responses in response to ConA stimulation.

TABLE 1

| | in vitro stimulation (hrs) | | | |
|---|---|---|---|---|
| Cytokine | | | | |
| Immunization | | | | |
| (IP) | None | None | None | ConA |
| | (24 hrs) | (72 hrs) | (120 hrs) | (72 hrs) |
| IL-2 (pg/ml) | | | | |
| Saline | ND | ND | ND | ND |
| HSA | ND | ND | ND | ND |
| LNnT-HSA | 209 ± 68* | 2,060 ± 286* | 63 ± 1* | ND |
| IL-4 (pg/ml) | | | | |
| Saline | ND | ND | ND | 76.56 ± 18.28 |
| HSA | ND | ND | ND | 31.81 ± 3.64 |
| LNnT-HSA | 63.74 ± 24.11* | 479.07 ± 86.80* | 1,363.33 ± 259.70* | 234.00 ± 28.15* |
| IL-5 (pg/ml) | | | | |
| Saline | 48 ± 4 | 48 ± 2 | 49 ± 1 | 81 ± 2 |
| HSA | 43 ± 2 | 42 ± 1 | 45 ± 6 | 78 ± 6 |
| LNnT-HSA | 178 ± 5* | 808 ± 93* | 3,018 ± 604* | 671 ± 34* |
| IL-10 (pg/ml) | | | | |
| Saline | ND | ND | ND | ND |
| HSA | ND | ND | ND | ND |
| LNnT-HSA | ND | 1,068 ± 223* | 7,064 ± 1,381* | 1,529 ± 361* |
| IFN-g (pg/ml) | | | | |
| Saline | ND | ND | ND | 3577 ± 510 |
| HSA | ND | ND | ND | 3358 ± 471 |
| LNnT-HSA | ND | 2090 ± 891* | 12,220 ± 1251* | 5917 ± 1132 |

Selective In Vitro B7-2 (CD86) Expression on B220+ Cells in LNnT-Inoculated Mice

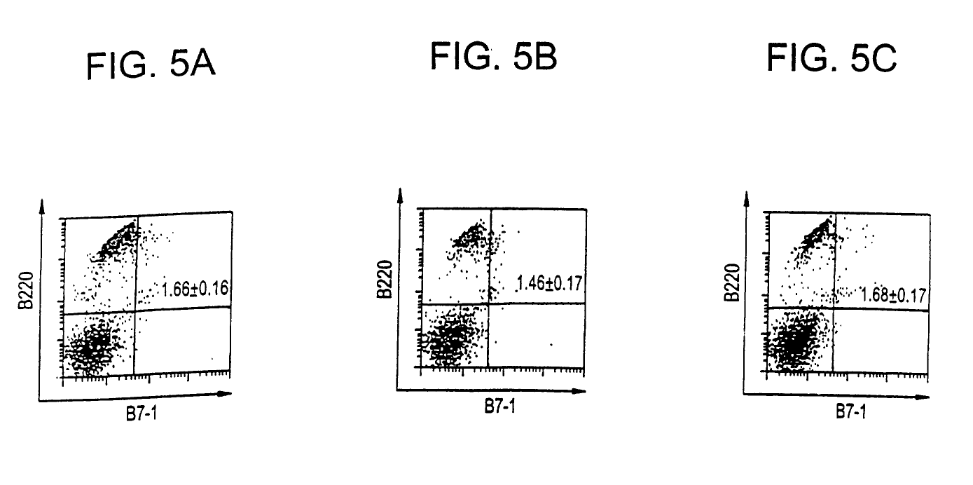

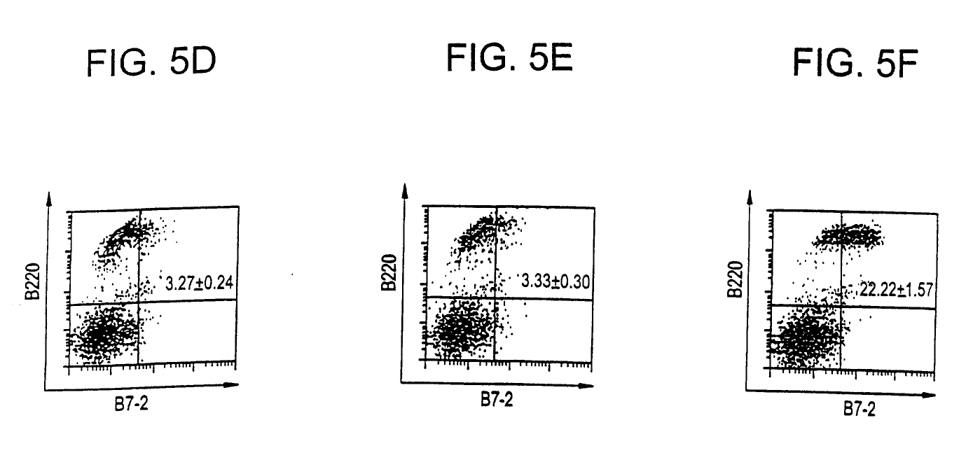

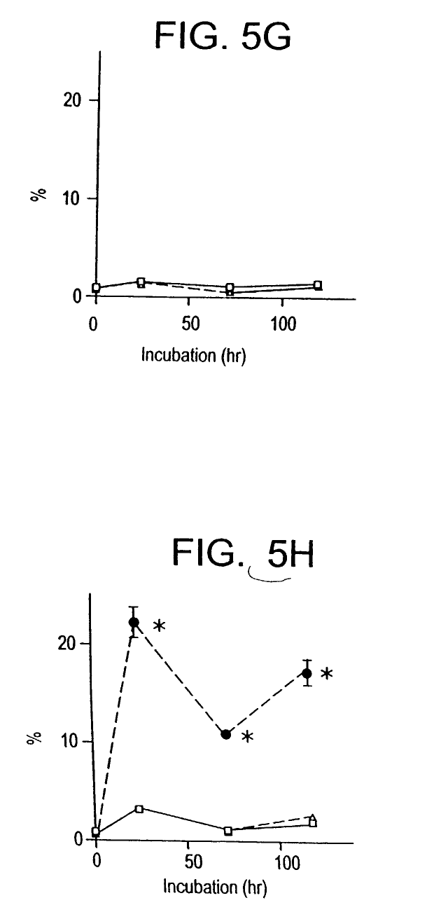

The expression of the costimulatory molecules, B7-1 and B7-2 on B220 positive splenocytes was also investigated. In freshly isolated splenocytes, the percentage of the cells positive for both B7-1 and B220 molecules were 1.02±0.08, 0.95±0.07, and 0.88±0.06 in mice IP immunized with saline, HSA, and LNnT13-HSA, respectively (n=6). The percentage of the cells positive for both B7-2 and B220 molecules were 0.59±0.04, 0.59±0.05, and 0.56±0.04 in mice IP immunized with saline, HSA, and LNnT13-HSA, respectively, suggesting no difference of the expression of these molecules in freshly isolated splenocytes. B7-1 expression was not altered in cultured splenocytes (FIGS. 5a,c,e). However, following 24 hrs culture incubation without restimulation in vitro, B220 positive cells expressing B7-2 molecule were increased in mice IP immunized with LNnT13-HSA compared to the control mice immunized with saline or HSA (FIGS. 5b.d.f). This increase was found following 6 hr incubation, and lasted at least 120 hrs, although B7-1 expression on B220 positive cells was not altered throughout the period observed (FIGS. 5g,h).

IL-4 is Required for the Induction of Polyclonal IgE Production by Multivalent LNnT

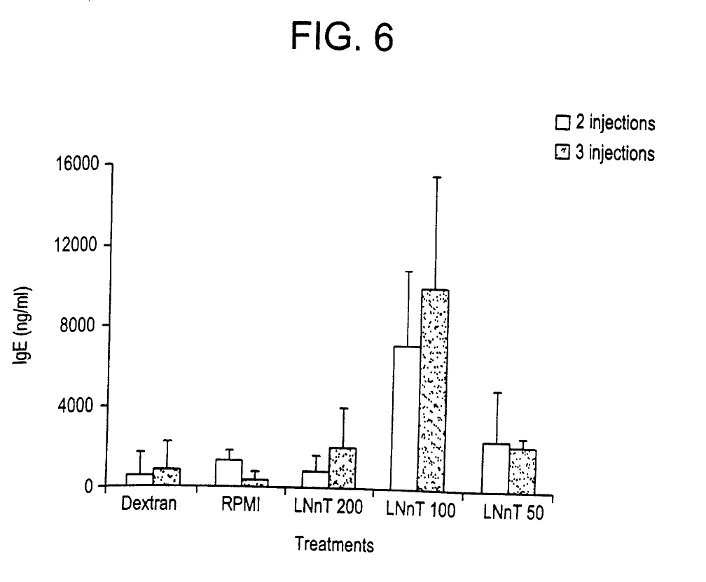

Because Th-2 cytokines, especially IL-4, are involved with IgE production, the role of IL-4 for the induction of polyclonal IgE production by multivalent LNnT in vivo was investigated using IL-4 gene deficient mice. IL-4 deficient mice did not induce polyclonal IgE production following repeated IP immunization with LNnT13-HSA. In this experiment, total serum IgE. IL-4 deficient mice or wild type BALB/.C mice were immunized with saline, HSA or LNnT13-HSA. Following second boosting immunization, sera were sampled and serum total IgE was measured. Following second boosting immunization, 2.5×10⁶/ml splenocytes from wild type or IL-4 deficient mice were cultured without additional stimulants and the production of IL-4, IL-5, IL-10, and IFN-γ were measured at 24, 72, and 120 hrs postincubation. Interestingly, splenocytes from IL-4 deficient mice with the immunization of LNnT13-HSA produced IL-5, IL-6, and IFN-γ without restimulation. However, the amounts of IL-5 and IL-6 were significantly lower than wild type BALB/C mice (FIGS. 6b,c,d,e). These cytokines were not detected in IL-4 deficient mice IP immunized with saline or HSA, indicating that IL-4 is required for the induction of polyclonal IgE by multivalent LNnT.

### Example 2

Additional experiments demonstrating the induction of polyclonal IgE by multivalent LNnT were performed, as described below.

In a first series of experiments, mice were injected intraperitoneally either two or three times with either dextran alone, RPMI media or LNnT conjugated to dextran in saline (LNnT-dextran). The sugar conjugate was referred to as LNnT35, wherein the 35 refers to the degree of LNnT substitution on each dextran molecule. The total amount of IgE elicited in the mice was determined. The results are shown in FIG. 6, wherein the numbers after LNnT (i.e., 200, 100 or 50) refer to the amount injected (dextran weight). The results demonstrate that the LNnT-dextran construct is able to raise large amounts of total IgE in vivo.

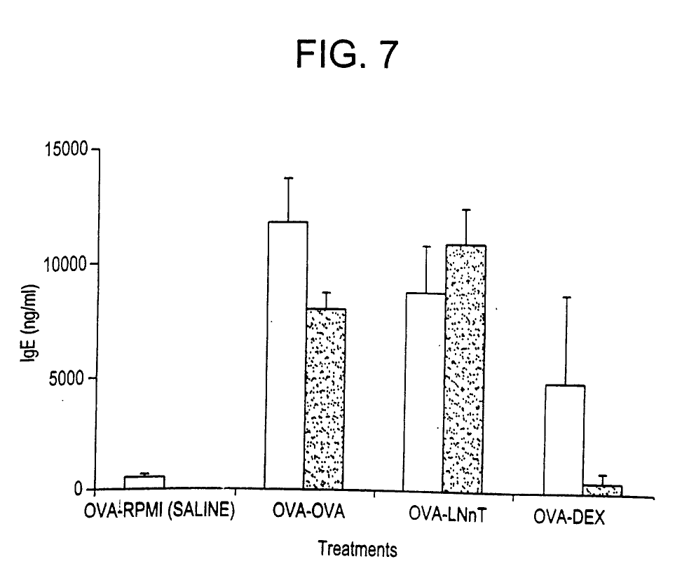

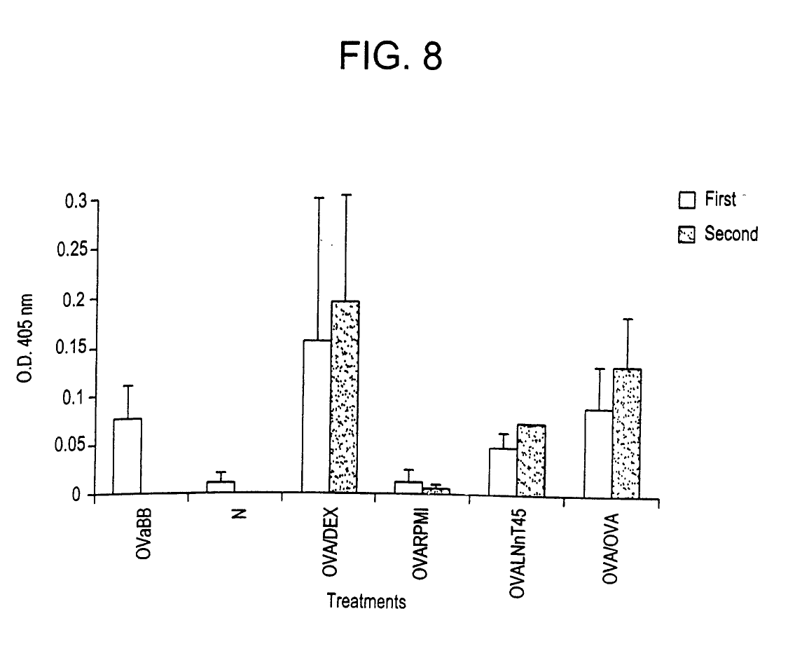

In another series of experiments, the effect of pre-treatment with multivalent LNnT on the production of antigen-specific IgE was investigated. Mice were injected either with ovalbumin (as a specific antigen) followed by a second injection of ovalbumin, or with the dextran conjugate LNnT45 followed by ovalbumin. Controls were mice receiving RPMI or dextran, followed by ovalbumin. FIG. 7 shows the total IgE in the mice and FIG. 8 shows the ovalbumin-specific IgE in the mice. FIG. 7 demonstrates that there was no discernible difference in levels of total IgE in the mice. FIG. 8 demonstrates, however, that prior treatment with the LNnT dextran conjugate does in fact reduce the amount of OVA-specific IgE by 40-50%. Thus, the LNnT conjugate functions to reduce the allergen specific IgE in vivo.

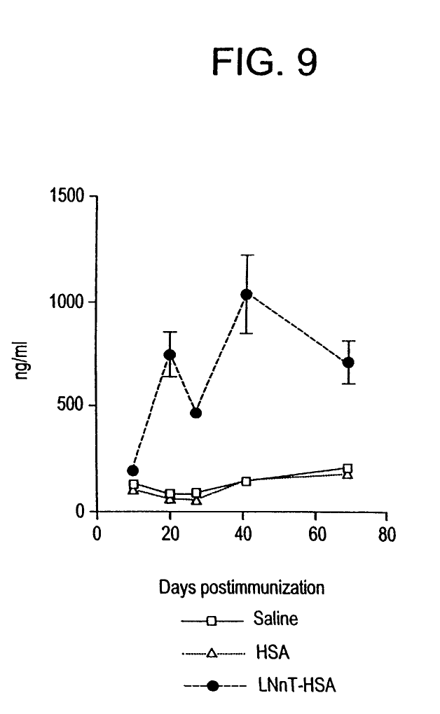

In another experiment, the length of time that non-specific IgE levels remain elevated after treatment with multivalent LNnT was investigated. LNnT-HSA was used as the conjugate, with saline and HSA alone serving as controls. The results are shown in FIG. 9, which demonstrates that mice treated with LNnT-HSA exhibited high levels of non-specific IgE for at least 70 days (the longest time point in the study). Thus, treatment with multivalent LNnT leads to persistent non-specific IgE.

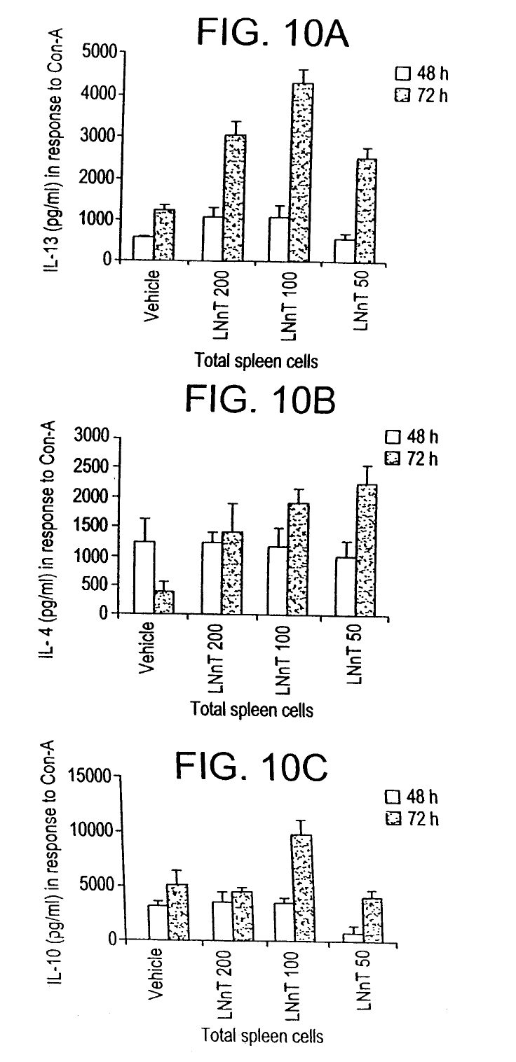

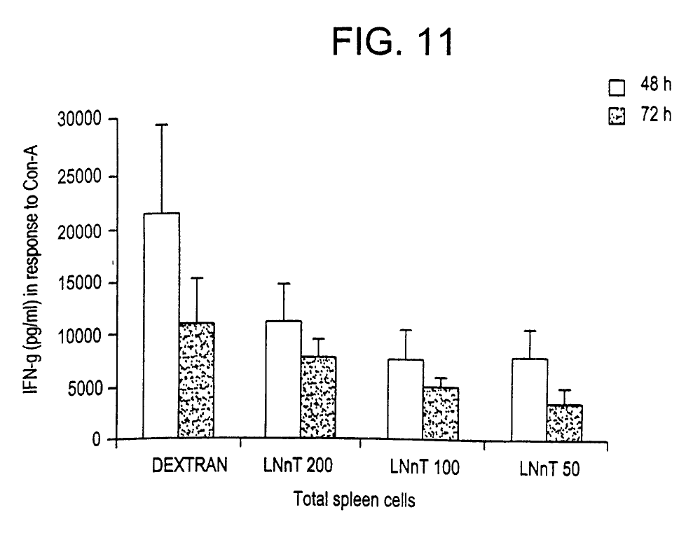

In another experiment, the effect of treatment with LNnT-dextran on production of either Th2-type cytokines or Th1-type cytokines was examined. Mice were immunized intraperitoneally with LNnT-dextran, total splenocytes were harvested and stimulated with ConA, followed by measurement of cytokine levels at 48 or 72 hours of culture. The results for Th2-type cytokines are shown in FIG. 10, which demonstrates that LNnT-dextran treatment leads to significantly elevated levels of IL-10, IL-4 and IL-13 at 72 hours culture compared to vehicle (dextran) immunized controls. The results for the Th1-type cytokine interferon-gamma are shown in FIG. 11, which demonstrates that LNnT-dextran treatment reduces the level of interferon-gamma at both 48 and 72 hours.

Discussion

These examples teach that multivalent LNnT induces polyclonal IgE production in mice by repeating IP immunization in vivo. Despite the fact that several reports have demonstrated polyclonal IgE production in mice and human in vivo and in vitro by the extracts from the parasites, the mechanism for promoting polyclonal IgE production by parasites is yet unclear (Wang, M Q. et al. (1995) Parasite Immunol. vol. 17 pp. 609-615; McGibbon, A M. et al. (1990) Mol. Biochem. Parasitol. vol. 39 pp. 163-172; Lee, T D G. et al. (1995) J. Allergy Clin. Immunol. vol. 95 pp. 124-1254Yarnashita, T. et al. (1993) Immunology vol. 79 pp. 185-195. Yamashita, U. et al. (1993) Jap. J. Parasitol. vol. 42 pp. 211-219). In fact, B cells are nonspecifically activated in parasite-infected host (Fisher, E. et al. (1981) Clin. Exp. Immunol. vol. 46 pp. 89-97). And carbohydrate moieties on schistosoma japonicum egg antigen is reported to activate not only schistosome-primed but also naive B cells (Yarnashita, T. et al. (1993) Immunology vol. 79 pp. 185-195). Therefore it seems that schistosomal antigens, especially carbohydrate moieties, have a mitogenic effect against B cells to induce nonspecific activation. Indeed, there are reports that the extracts from nematodes contain a B cell mitogen that regulate nonspecific B cell activation to induce polyclonal IgE production (Wang, M Q. et al. (1995) Parasite Immunol. vol. 17 pp. 609-615; Lee, T D G. et al. (1995) J. Allergy Clin. Immunol. vol. 95 pp. 124-1254). However, B cell mitogenic activity is not enough to promote polyclonal IgE production.

It is known that IL-4 induces B cells to develop polyclonal IgE producing cells in mice in vivo and in vitro (Coffman, R L. et al. (1986) J. Immunol. vol. 136 pp. 949-954; Finkelman, F D et al. (1990) Annu. Rev. Immunol. vol. 8 pp. 303-333; Tepper, R I. et al. (1990) Cell vol. 62 pp. 457; Snapper, C M. et al. (1991) J. Immunol. vol. 147 pp. 1163-1170; Nakanishi, K. et al. (1995) Int. Immunol. vol. 7 pp. 259-268). Therefore the help of IL-4 derived from IL-4 producing cells such as T cells (including NK1⁺ CD4⁺ T cells), eosinophils, and cells of the mast cell/basophil lineage may be required (Coffman, R L. et al. (1997) J. Exp. Med. vol. 185 pp. 373-375; Sabin, E A. et al. (1996) J. Exp. Med. vol. 184 pp. 1871-1878). Thus, at least two factors in parasite antigens may be required to induce polyclonal IgE production: B cell mitogenic activity and induction of IL-4 production. Lee et al. showed that the body fluid of Ascaris is capable of increasing total IgE levels in mice by a single subcutaneous injection (McGibbon, A M. et al. (1990) Mol. Biochem. Parasitol. vol. 39 pp. 163-172; Lee, T D G. et al. (1995) J. Allergy Clin. Immunol. vol. 95 pp. 124-1254; Lee, T D G. et al. (1993) Int. Arch. Allergy Immunol. vol. 102 pp. 185-190). However, they did not isolate the molecule that induce IgE production, but showed that ABA-1, the purified major allergen of Ascaris did not induce the increase of total IgE. Thus they suggested the model of polyclonal IgE induction by nematode that nematode products contain a B-cell mitogen that polyclonally activates B cells, which is converted into a polyclonal IgE response when these stimulated B cells come under the influence of IL-4 or an IL-4 like molecule activated by other factors.

Splenocytes from mice IP immunized with multivalent LNnT produced IL-4, IL-5, and IL-10 without restimulation in vitro, although they also produced detectable amount of IL-2 and IFN-γ. In addition, IL-4 deficient mice did not induce polyclonal IgE production following IP immunization with this carbohydrate although they produced significant amount of IL-5, IL-10, and IFN-γ. Development of IL-5 and IL-10 production following S. mansoni infection was also seen in IL-4 deficient mice (Pearce, E J. et al. (1996) Int. Immunol. vol. 8 pp. 435-444; King., C L. et al. (1996) Exp. Parasitol. vol. 84 pp. 245-252). These results indicate that IL-4 is required for inducing polyclonal IgE production by multivalent LNnT in vivo. Moreover, splenocytes from mice IP immunized with multivalent LNnT were found to produce significantly more IL-4 in response to ConA, the T cell mitogen. This result is consistent with the reports that schistosome-infected host shows mitogen-driven IL-4 production and the production is correlated to serum total IgE (King, C L. et al., (1993), J. Immunol. vol. 150 pp. 1873-1880; Ogilvie B M Nature 1964 204. 91-92; Zwingenberger, K. et al. (1991) Scand. J. Immunol. vol. 34 pp. 243-251). Therefore, multivalent LNnT likely skews host susceptible to produce IL-4. The source of IL-4 was analyzed by intracellular staining in in vitro culture, and it was demonstrated that CD4+ T cells are responsible for the production of IL-4.

Collecting these findings, multivalent LNnT may possess at least two functions, B cell mitogenic activity and induction of IL-4 production, to induce polyclonal IgE production. In fact, splenocyte from control mice IP immunized with saline also showed the significant proliferative responses against multivalent LNnT, suggesting that this carbohydrate possesses the mitogenic activity.

CBA/J and BALB/C mice produce the polyclonal IgE following second immunization, on the other hand, C57BL/6 mice produce it following third immunization. It is known that host response against S. mansoni infection is strain dependent. CBA/J, C3H/HeJ, and BALB/C mice developed bigger liver granulomas and higher portal hypertension whereas C57BL/6 mice developed relatively smaller granulomas and lower portal hypertension (Fanning, M M. et al. (1981) J. Inf. Dis. vol. 144 148-153; Hernandez, N J. et al. (1997) Eur. J. Immunol. vol. 27 pp. 666-670). Immunization with LNnT seems to have a same characteristics as S. mansoni infection in terms of the susceptible strain of mice, suggesting that this carbohydrate may be a dominant putative antigen in S. mansoni. Amiri et al. demonstrated that complete suppression of the total IgE response resulted in the decreases in worm burden and egg production in S. mansoni-infected normal and IFN-γ knockout mice (Amiri, P. et al. (1993) J. Exp. Med. vol.180 pp. 43-51). The present result may relate with these reports that in primary S. mansoni infection, C57BL/6 mice produced relatively lower amount of polyclonal IgE production in response to carbohydrate antigen in S. mansoni, results in the decreased egg production and worm burden (Amiri, P. et al. (1992) Nature vol. 356 pp. 604).

In addition, it has been previously demonstrated that peritoneal B-1 cell outgrowth due to S. mansoni infection was strain dependent, occurring in CBA/J, C3H/HeJ, and BALB/C mice but not in C57BL/6 mice (Palanivel, V. et al. (1996) Exp. Parasitol. vol. 84 pp. 168-177). B-1 cell subset is a major source of B cell IL-10 that downregulate Th1 responses (Amiri, P. et al. (1992) Nature vol. 356 pp. 604). The present result that peritoneal B-1 cells seem to be involve in part in the induction of polyclonal IgE by multivalent LNnT (FIG. 2d) is consist with the report.

Polyclonal IgE production in response to multivalent LNnT is not due to LPS contamination, because mice IP immunized with LNnT-HSA produced same amount of serum IgG3 as compared with controls. It is known that LPS induced IgG3 production both in vivo and in vitro (Coffman, R L. et al. (1986) J. Immunol. vol. 136 pp. 949-954; Finkelman, F D et al. (1990) Annu. Rev. Immunol. vol. 8 pp. 303-333). And LPS itself did not induce IL-4 production in vitro, whereas splenocytes from mice IP immunized with LNnT-HSA did produce IL-4.

Following in vitro culture incubation without restimulation, B7-2 positive cells were increased in B220+ cell population in mice IP immunized with LNnT-HSA compared to the control mice immunized with saline or HSA. B7-1 and B7-2 costimulatory molecules are ligands for CD28/CTLA-4 and involved in T cell activation, cytokine production, and regulation of tolerance (McKnight, A J. et al. (1994) J. Immunol. vol. 152 pp. 5220-5225; Perez, V L. et al. (1997) Immunity vol. 6 pp. 411-417). Costimulation by B7-1 and B7-2 can differentially regulate Th1 cell differentiation, although the effect of these molecules are dependent on the status of immune reaction, doses and routes of antigen inoculation, types of APC, and the experimental model of diseases (Thompson, C B. (1995). Cell vol. 81 pp. 979-982). For example, in studies of experimental allergic encephalomyelitis (EAE) in mice, administration with anti-B7-1 diminished the severity of neurologic disease, which is mediated by Th1 cells, while anti-B7-2 administration enhanced the severity (Kuchroo, V K. et al. (1995) Cell vol. 80 pp. 707-718). Recent reports suggest that B7-2 may play a critical role in the ability to initiate a Th2 response (Thompson, C B. (1995). Cell vol. 81 pp. 979-982; McArthur, J G. et al. (1993) J. Exp. Med. vol. 178 pp. 1645-1653). The present results are consistent with these reports and suggest that B7-2 expression is closely associated with polyclonal IgE production by multivalent LNnT. However, freshly isolated B220+ cells from mice IP immunized with multivalent LNnT did not express the significant levels of B7-2 compared with those from control mice. This result means that LNnT indirectly induce the B7-2 expression on B220+ cells. This may be due to IL-4 secreted by Th2 cells, because IL-4 deficient mice did not induce B7-2 expression. Stack et al. demonstrated that IL-4 treatment of small splenic B cells induced both B7-1 and B7-2 molecules (Stack, R M. et al. (1994) J. Immunol. vol. 152 pp. 5723-5733. They also reported that B7-2 expression was detected at 6 hr and appeared to be maximal at 24 hr, whereas B7-1 was not observed until 48 hr and was maximal at 72 hr. B7-1 expression could not be detected until 120 hr. This difference may be due to the status of B cells. This result represents the expression of primed B220+ cells by multivalent LNnT, on the contrary, the authors investigated the resting B cells.

The current findings suggest that in vivo induction of polyclonal IgE production by multivalent LNnT may be useful for immunotherapy or prophylaxis of allergic and anaphylactic reaction in which detrimental chemical mediators are released from effector cells by the crosslink of antigen-specific IgE on the cell surface. Further, these results encourage us to apply for the reduction of anaphylactic reaction in not only parasite infection but also environmental allergy (Hagel, I. et al. (1993) Parasite Immunol. vol. 15 pp. 311-315).

### Example 3

### In Vivo Stimulation with LNnT-Dextran Conjugate Promotes Th2 and Suppresses Th1 Responses

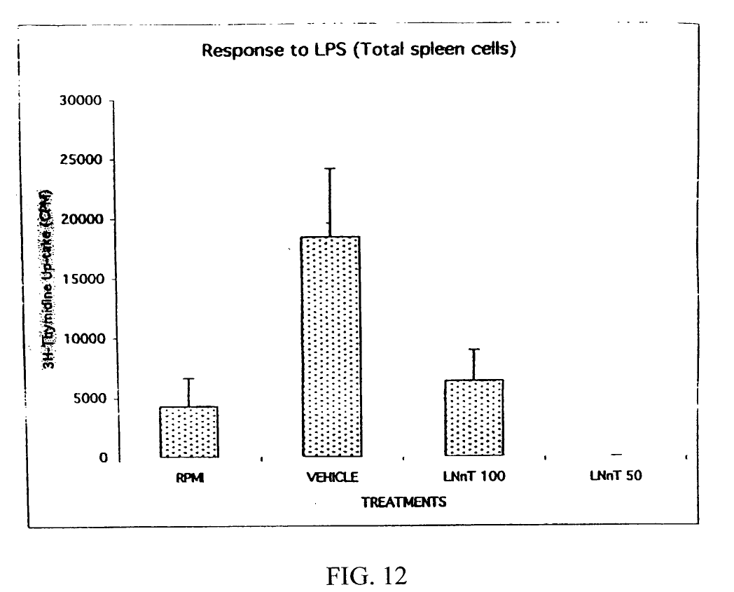

In this example, mice were injected intraperitoneally with either media alone (RPMI), vehicle alone (dextran), 50 μg of LNnT-dextran conjugate (LNnT 50) or 100 μg of LNnT-dextran conjugate weekly for three weeks. The spleen cells were then harvested from the mice and either total spleen cells or CD4+ cells were stimulated in vitro with ConA or LPS. Following in vitro stimulation, the proliferative and cytokine responses of the cells were measured. The results are illustrated in FIGS. 12-16.

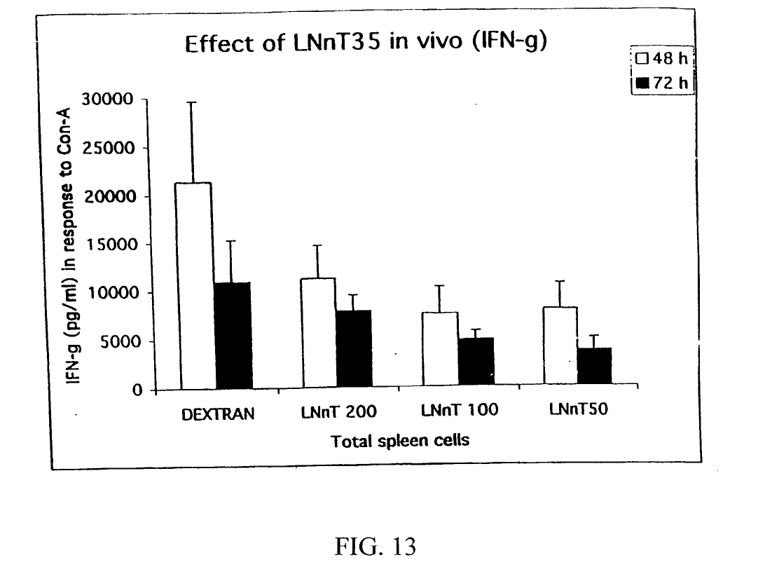

FIG. 12 demonstrates that the proliferative response of total spleen cells from the LNnT-dextran treated mice following in vitro LPS stimulation was significantly decreased as compared to mice treated only with vehicle (dextran). FIG. 13 demonstrates that interferon-gamma production (after 48 or 72 hours) by total spleen cells from the LNnT-dextran treated mice (treated in vivo with either 200, 100 or 50 μg of conjugate) following in vitro ConA stimulation was significantly decreased as compared to mice treated only with vehicle (dextran).

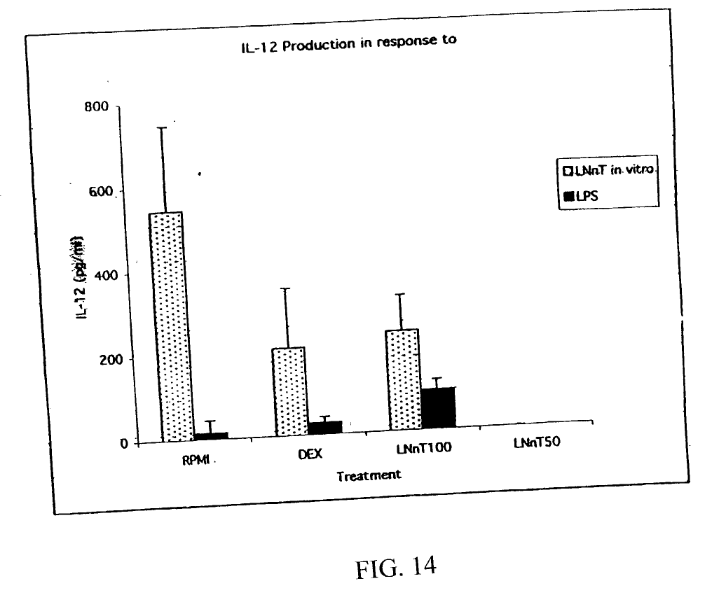

In the experiment for which the results are depicted in FIG. 14, the mice were treated with media alone (RPMI), vehicle (dextran) or LNnT-Dex at either 100 μg or 50 μg doses. The spleen cells were taken from these groups of mice and then stimulated in vitro with either LNnT-dextran or LPS. The results illustrated in FIG. 14 demonstrate that in virtually naïve animals there is an initial burst of IL-12 production, which goes away and is actually suppressed in LNnT injected animals, especially at lower doses (compare RPMI and LNnT-Dex 50 groups).

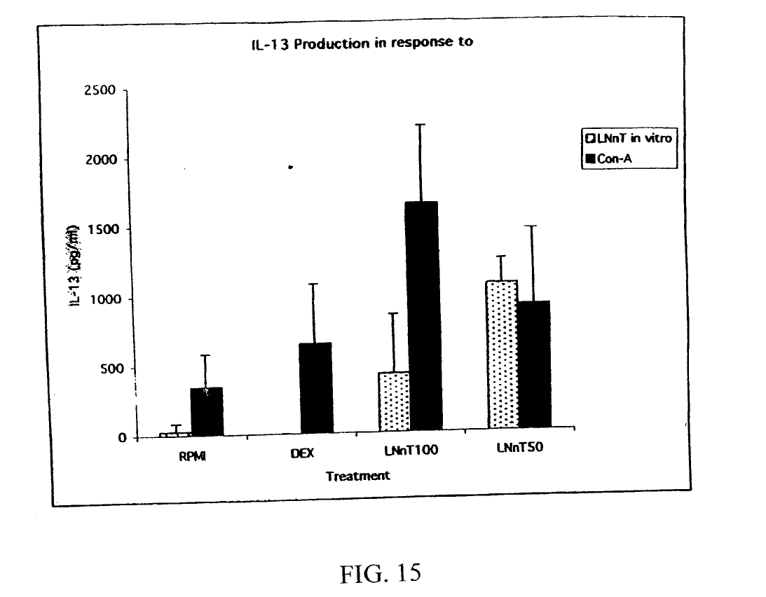

In the experiment for which the results are depicted in FIG. 15, the mice were treated with media alone (RPMI), vehicle (dextran) or LNnT-Dex at either 100 μg or 50 μg doses. The spleen cells were taken from these groups of mice and then stimulated in vitro with either LNnT-dextran or ConA. FIG. 15 demonstrates that IL-13 production by total spleen cells from the LNnT-dextran treated mice following in vitro stimulation with either LNnT-dex or ConA was significantly greater than IL-13 production by mice treated with vehicle (dextran) alone and then stimulated in vitro with LNnT-dex or ConA.

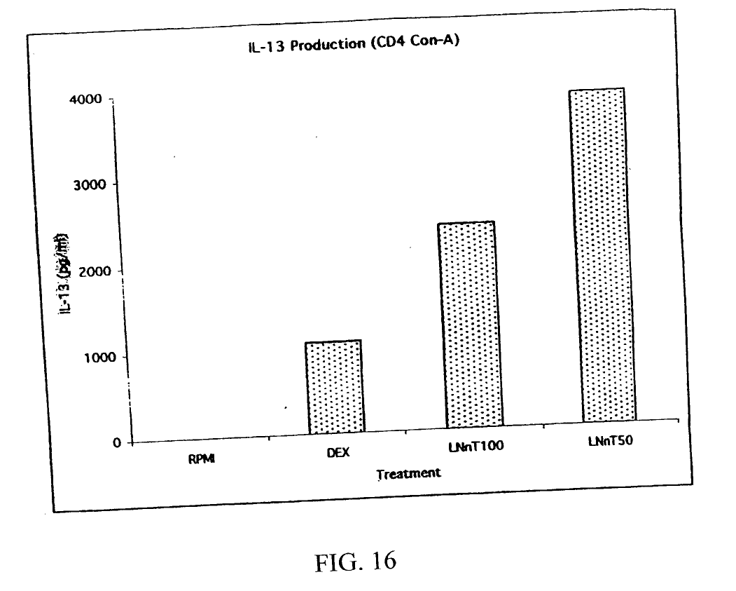

FIG. 16 demonstrates that IL-13 production by CD4+ cells from the LNnT-dextran treated mice following in vitro stimulation with ConA was significantly greater than IL-13 production by mice treated with vehicle (dextran) alone and then stimulated in vitro with ConA.

In summary, the data presented in FIGS. 12-16 demonstrate that spleen cells from LNnT-dex injected mice have suppressed responses to LPS and ConA as measured by reduced proliferation, reduced IL-12 production and reduced IFN-γ production. In contrast, production of IL-13 is elevated. Taken together, these data demonstrate that multivalent conjugates containing LNnT suppress type-1 immune responses elicited by strong mitogenic stimuli.

### Example 4

### LNnT-Dex Conjugates Recruit Peritoneal Exudate Cells That Resemble “Natural Suppressors”

Mice were treated with LNnT-dextran conjugate as described in Example 3 and peritoneal exudate cells (PECs) were recovered and analyzed by FACS analysis to characterize the surface markers expressed on this population of cells. The results demonstrated that intraperitoneal injection of the LNnT-Dex conjugate recruits a population of cells that are Gr1+, CD11b+ (but are CD11a- and CD11c-). In one experiment, these cells accounted for 30-40% of PECs in immunized mice compared to less than 4% in saline or dextran injected controls. These cells are seen as early as 2 hours post-injection.

To further characterize this population of PECs, similar FACS analyses were performed on PECs from both wild-type mice and Stat 6 knockout (KO) mice treated with LNnT-dextran. The data showed that injection of LNnT-Dex recruited about 19% Gr1+, CD11b+ cells in the peritoneal cavity after two hours in the Stat 6 KO mice and about 18.0-24% Gr1+, CD11b+ cells in the peritoneal cavity after two hours in the wild type mice. In contrast, injection of dextran alone in wild-type mice yielded only 5.5% Gr1+, CD11b+ cells. The ability to recruit the Gr1+, CD11b+ population of cells in the Stat 6 KO mice indicates that this induction is not IL-4 or IL-13 dependent. Furthermore, while the Gr1+ phenotype suggests granulocytes, this antibody is not very specific and cytospin analysis showed the population of cells to be less than 8% granulocytes, the remainder appearing as mononuclear cells. Staining with the murine macrophage specific monoclonal antibody F4/80 was negative. This cumulative data distinguish the population of cells that are recruited by LNnT-dex treatment from another population of cells called “alternatively activated macrophages.” It has been shown that the induction of “alternatively activated macrophages” requires IL-4 and that the cells are F4/80+, whereas the population of cells that are recruited by LNnT-dex treatment are F4/80− and do not require IL-4 for their induction.

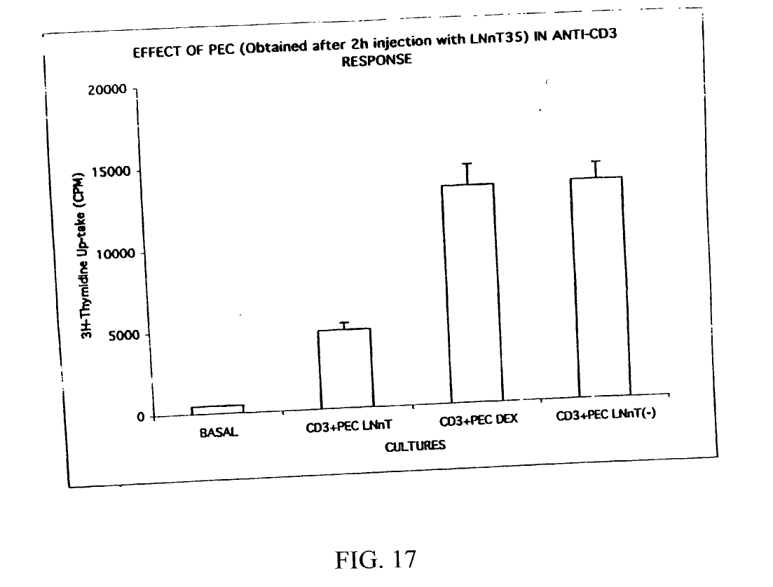

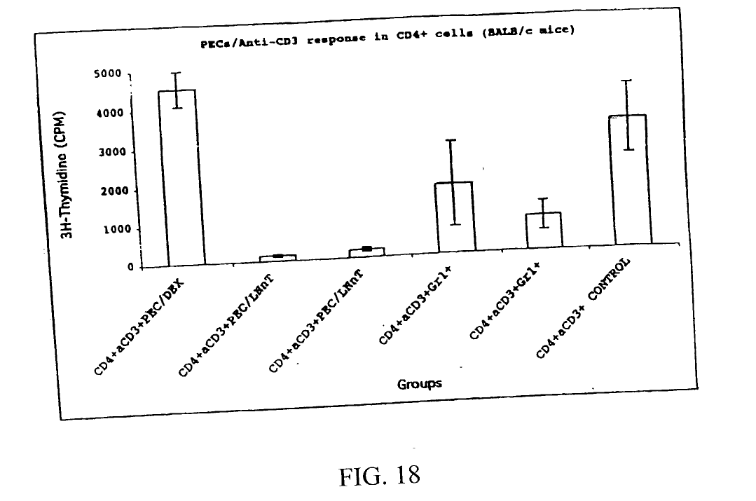

To examine the suppressor activity of the LNnT-activated PECs, mice were injected i.p. with either LNnT-dex, dextran alone or saline and two hours later PECs were harvested. Total naïve spleen cells were planted on wells coated with anti-CD3 antibodies and then the LNnT-activated PECs were added to the culture. The proliferative response of the naïve spleen cells was determined as a measure of their activation by anti-CD3. The results are illustrated in FIGS. 17 and 18.

FIG. 17 demonstrates that PECs obtained 2 hours post-injection of LNnT-dex are able to inhibit the proliferative responses of naive spleen cells stimulated with anti-CD3. (The data labeled CD3+ PEC LNnT(−) represents PECs from LNnT-dex injected mice which have had the Gr1+ cells removed; i.e., this data represents a Gr1− population of PECs).

FIG. 18 demonstrates that anti-CD3 proliferative responses by CD4+ cells from Balb/c mice are inhibited by PECs from LNnT-dex treated mice. (The data labeled CD4+ aCD3+Gr1+ represents PECs from LNnT-dex injected mice which have had enriched for Gr1+ cells).

In summary, the above data demonstrates that multivalent LNnT recruits a population of cells that are Gr1+, CD11b+, but that do not require IL-4 or IL-13 for their induction and that are F4/80−, wherein this population of cells has suppressor activity as evidenced by their ability to inhibit anti-CD3 proliferative responses.

### Example 5

### LNn-T Expands Gr-1⁺ Suppressor Cells That Secrete Anti-Inflammatory Cytokines and Inhibit Proliferation of CD4+ Cells

The inhibition of T cell responses has been attributed to the presence of a suppressor cell population identified phenotypically as Gr-1⁺/CD11b⁺. These cells have been termed natural suppressor cells or immature myeloid cells and appear to be mediating immunosuppressive effects in a wide variety of unrelated pathological conditions (23-27). To investigate whether the immune biasing provoked by polyvalent LNnT-Dex injection is related to a specific cell population, a series of ex-vivo experiments were conducted to decipher the early response to LNnT-Dex glycoconjugates. The early response to LNn-T-Dex in naïve mice was examined using a construct having 21 or more molecules of LNn-T conjugated to dextran.

Mice

Six-Ten week old female BALB/c and C57BL/6 mice were purchased from Jackson Laboratories (Bar Harbor, Me.) and were maintained in a pathogen free environment at the Harvard Medical School animal facility in accordance with institutional guidelines.

Preparation and Injection of Carbohydrates.

The Neo-glycoconjugate Lacto-N-neotetraose (LNnT-Dex) conjugated to a 10KDa dextran backbone was supplied by Neose Technologies Inc (Horsham, Pa.). The level of LNnT-Dex substitution varied from 21 to 45 LNnT-Dex residues per molecule of dextran.

LNnT-Dex 21, 35 or 45, diluted in saline, was administered at a dose of 100 μg/mouse in all experiments. Naive BALB/c or C57BL/6 mice were injected i.p. with the LNnT-Dex, dextran or saline and animals were sacrificed at 2 and 18 h later by CO₂ inhalation.

Peritoneal exudate cells (PECs) were obtained at 2 and 18 h post-injection by peritoneal lavage with 5 ml of ice-cold Hank's balanced salt solution (HBSS, Gibco). PECs were washed 2 times and red blood cells were lysed by hypotonic shock with ammonium chloride. Viable cells were counted and adjusted to 5×10⁵ cells/ml. Viability measured by trypan blue exclusion was routinely over 95%.

PECs were analyzed for surface markers, cytokine production and for suppressor activity in co-cultures with naive CD4 cells.

Flow Cytometric Analysis.

Peritoneal exudate cells were blocked with anti-mouse FcγR antibody (CD16/CD32) and stained with fluorescein isothiocyanate (FITC)-conjugated monoclonal antibodies against Mac-1 (CD11b), F4/80, Gr-1 (Ly-6G), M HC-II, B7-2 or phycoeritrin (PE)-conjugated antibodies against Gr-1 (Ly-6G), and IL-10R. All antibodies were purchased from Pharmingen (San Diego, Calif.), except anti-F4/80, which was obtained from Serotec (England). Stained cells were analyzed on a FACSCalibur using Cell Quest software (Becton Dickinson). Live cells were electronically gated using forward and side scatter parameters.

Cell Culture and Co-Culture of PECs-CD4+ Cells.

All cultures and co-cultures were maintained in RPMI 1640 (Gibco BRL) supplemented with 2 mM L-glutamine, 100 U/ml penicillin, 100 μg/ml streptomycin (Sigma), 5×10⁻⁵ M 2-β-mercaptoethanol (GIBCO BRL) and 10% fetal bovine serum (FBS, Hyclone).

PECs were adjusted to a concentration of 5×10⁵/ml, plated in 24 well plates (Costar) and were maintained at 37 C and 5% CO₂ for 72 h and the spontaneous production of cytokines was evaluated by ELISA. Supernatants were harvested, centrifuged and examined for IFN-γ, IL-12, IL-10 (antibodies and cytokines were obtained from Pharmingen), IL-1β, IL-18, IL-13, and TGF-β (obtained from R&D).

Co-culture of PECs with naive CD4⁺ cells was performed as follows: PECs were obtained as described and adjusted to 5×10⁵PECs/ml. Splenocytes were prepared from naive mice, and enriched for CD4+ cells (>95% by FACs analysis) using CD4 magnetic cell sorter beads (MACS, Miltenyi Biotec, Germany). CD4⁺ cells were plated in 96 well flat bottom plates (Costar, Cambridge, Mass.) which were pre-coated with anti-CD3 and anti-CD28 antibodies (Pharmingen) at 1 μg/ml. Three hours later PECs were added at ratios of 1:4-1:16 (PECs:CD4⁺) into cultures. Cultures were maintained at 37 C and 5% CO2 for 72 h, then ³H-Thymidine (185 GBb/mmol activity, Amersham, England) 1 μCi/well was added and incubated for a further 18 h. Cells were harvested on a 96 well harvester (Tomtec, Toku, Finland) then counted using a β-plate counter. Values are represented as CPM from triplicate wells. In some experiments supernatants from co-cultures were harvested and analyzed for IL-4, IL-13 and IFN-γ production.

Removal of Gr-1⁺ Cells.

The Gr-1⁺ cell population of PECs was removed using MACS beads. Briefly, PECs pooled from 4 mice were incubated with monoclonal anti-Gr-1 antibody (Pharmingen), for 30 min at 4 C, washed two times and re-incubated with paramagnetic beads (MiniMAcs, Miltenyi Biotec) coupled to goat anti-rat IgG (isotype of the anti-Gr-1 antibody) for 15 min at 4 C. PECs were washed twice and passed through a magnetic column (Miltenyi Biotec) to retain Gr-1⁺ cells. The eluted cell fraction (<3% Gr-1⁺ according to FACS analysis) was adjusted to 5×10⁵ cells/ml and used in the co-cultures as described above.

Fixed PECs, Transwells Cultures and Blocking Antibody Experiments.

In some co-cultures PECs were fixed with 0.5% paraformaldehyde for 5-10 min, washed extensively with RPMI, adjusted to 5×10⁵/ml and added in varying concentrations to 96-well plates in the presence of CD4+ naive cells previously stimulated with plate-bound anti-CD3/CD28 antibodies. These co-cultures were processed as described.

In others cultures 24 well (0.4 μm pore) transwell cell culture plates (Costar) were used to separate PECs from naive CD4⁺ cells. PECs were plated on the superior chamber at a ratio 1:4 to CD4⁺ cells, the inferior chamber was coated with anti-CD3/CD28 antibodies and naive CD4⁺ cells were added. Cultures were maintained for 72 h, then 1 μCi/well ³H-Thymidine added (Amersham) and incubated for an additional 18 h. CD4⁺ cells were transferred to a 96 well plate for harvesting as described.

The role of soluble factors in PEC suppression was assayed by using varying concentrations of isotype control or blocking antibodies ((anti-IL-10 (1, 2 and 5 μg/ml) and anti-TGF-β (2-5 μg/ml)) in 96-well plate co-cultures, where antibodies were added at the same time as PECs.

LNnT-Dex Injection Expands a Greater Number of PECs Than Injection with Control Dextran.

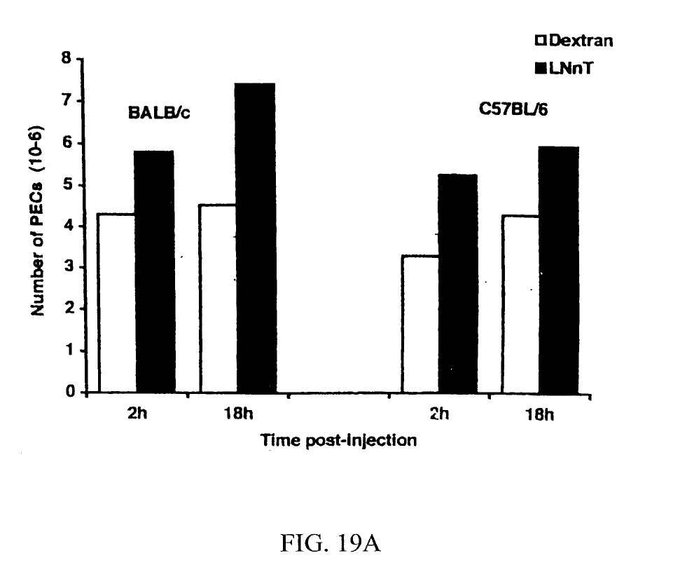

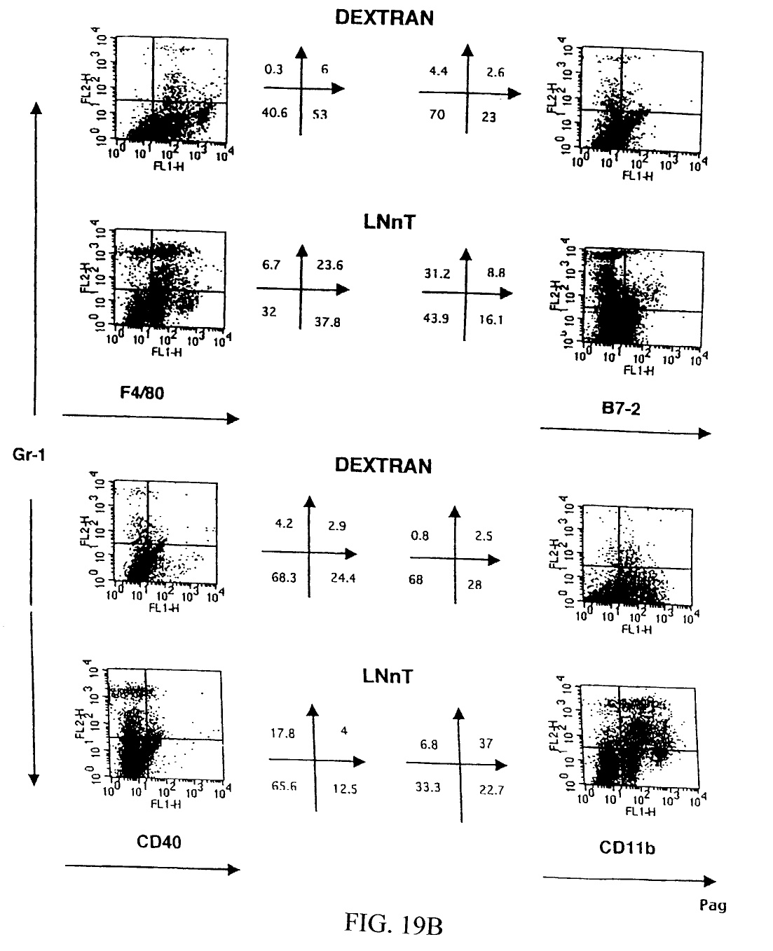

The peritoneal cell response in mice to the polyvalent sugar LNnT-Dex was examined at early (2 h) or late (18 h) time points after injection. The peritoneal cavity of animals was rinsed with 5 ml of cold HBSS and PECs harvested. FIG. 19a shows that at both time points animals receiving polyvalent LNnT-Dex rapidly expanded peritoneal cells compared to animals that received dextran, and that at 18 h after injection more PECs were recruited to the peritoneal cavity than at 2 h in LNnT-Dex injected mice.

Gr-1+/F4/80+/CD11b+ is the Predominant Cell Type Expanded by LNnT-Dex.

The composition of the PECs was analyzed by two-color flow cytometry. FIG. 19b summarizes the main surface markers detected in the population expanded by polyvalent LNnT-Dex. The majority of the PECs are Gr-1+/CD11b+ (ranging from 30 to 40% of the total cells, FIG. 19b) and F4/80+/Gr-1+ (in lower percentage, ranging 25-37%). In contrast, PECs recruited by dextran or saline did not show high levels of cells positive for Gr-1 (5-7%), but did express CD11b (28% or higher) and F4/80 (30-50%). Gr-1⁺ PECs from LNnT-Dex expressed low levels of B7-2, MHC-II and CD40, but did not express CD11c, IL-10 receptor or NK surface marker.

PECs Expanded by LNnT-Dex Exhibit “Natural Suppressor Cell” Activity.

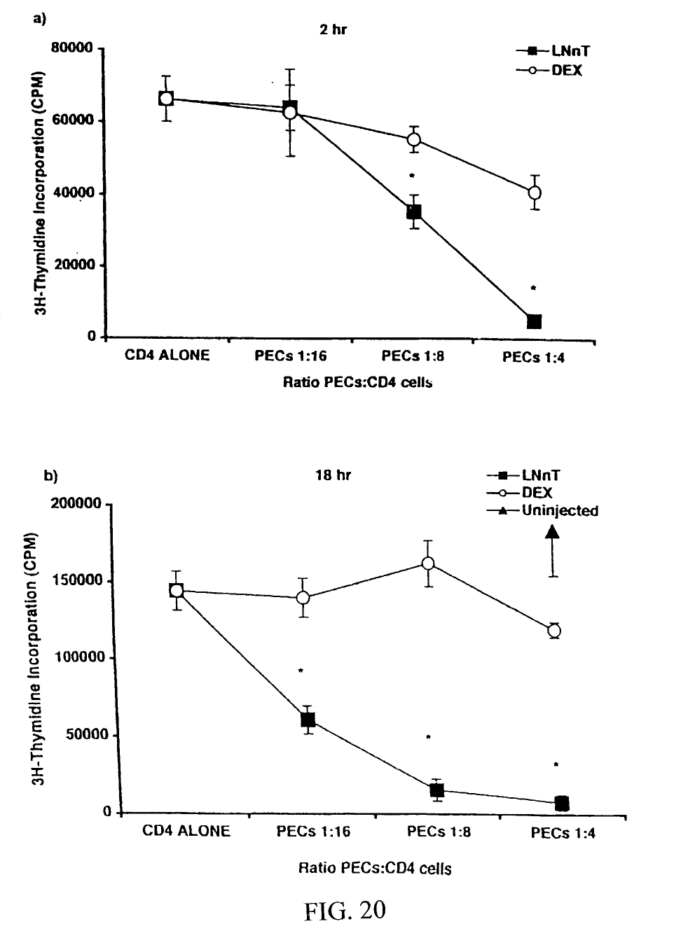

Recent reports in animals receiving chronic injections of virus (Bronte, et al. J. Immunol. 165:163:5728, 1999), or tumor bearing mice (Kusmartsev, et al., J. Immunol. 165:779, 2000) have shown the accumulation of Gr-1⁺/CD11b⁺ cell populations called “Natural Suppressors”. To determine the suppressive activity in PECs, co-cultures were performed with naive CD4⁺ T cells and PECs expanded by polyvalent LNnT-Dex, dextran, or saline. Naive CD4⁺ cells were stimulated by plate-bound anti-CD3 and anti-CD28 antibodies, and three hours later PECs in varying ratios were added to the cultures. FIG. 20 shows that CD4⁺ T cells in the absence of PECs, or in the presence of control PECs (saline or dextran) exhibited high levels of proliferation in response to anti-CD3/CD28 stimulation. In contrast, in the presence of PECs from LNnT-Dex injected mice, the CD4⁺ cells proliferated poorly, especially at higher PEC concentrations (FIGS. 20a-b). This phenomenon was observed in both strains of mice tested (BALB/c and C57BL/6) and was characteristic of both early (2 h) and late (18 h) times post-sugar injection. There was a significant inhibition in the proliferative response even when the PECs:CD4⁺ ratio was 1:8, with a 50% reduction of proliferation compared to controls. However, the clearest inhibition was detected using a ratio 1:4 where greater than 90% suppression was observed (FIGS. 20a-b).

Gr-1⁺ Cells Mediate Suppression Induced by PECs Elicited by LNnT-Dex.

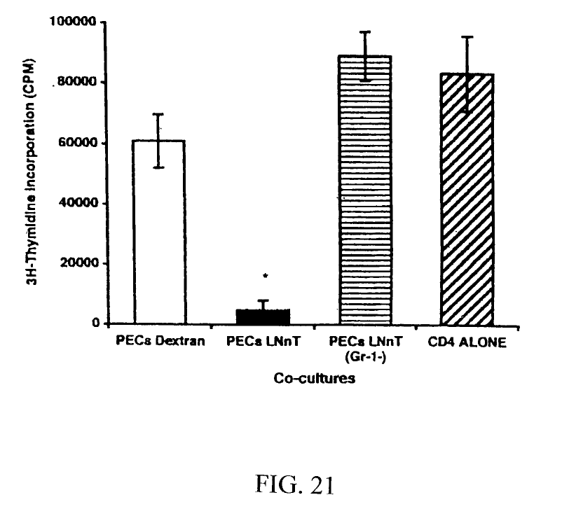

As shown in FIG. 20, polyvalent LNnT-Dex recruits PECs that specifically inhibits naive CD4⁺ cells proliferative response to anti-CD3/CD28 stimulation. To identify the cell type responsible for this suppressive activity, we depleted PECs of Gr-1⁺ cells (the most prominent cell marker detected) using magnetic cell sorter, and the negative fraction was used in PEC:CD4⁺ co-cultures. It was found that the suppressive activity previously observed in PECs recruited by LNnT-Dex was significantly abolished (p<0.01) in the absence of Gr-1⁺ cells (FIG. 21), indicating an important role for cell populations bearing this marker in suppression.

Suppression is Mediated by Both Cell to Cell Contact and by Soluble Factors.

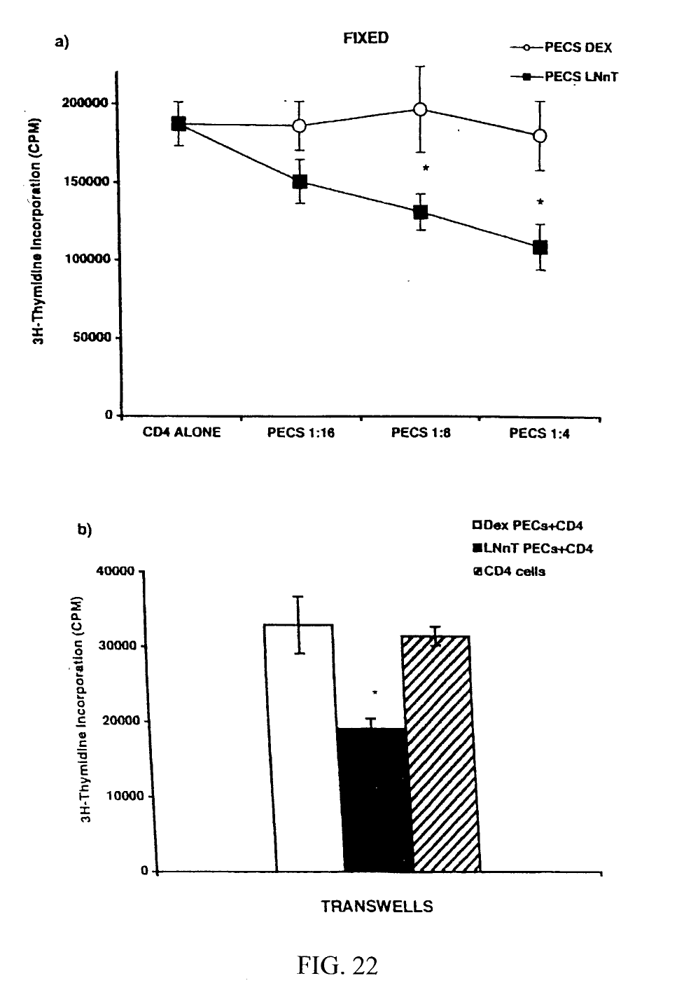

Fixed cells were tested to determine whether they could retain their suppressor activity. PECs from LNnT-Dex or dextran injected mice were harvested, adjusted to 5×10⁵/ml and fixed with 0.5% paraformaldehyde for 5-10 min, PECs were then washed and added to previously (3 h) stimulated naive CD4⁺ cells. Fixed LNnT-Dex recruited PECs retained some ability to inhibit the proliferation of CD4⁺ cells stimulated by anti-CD3/CD28 antibodies (FIG. 22a). As expected, the inhibition was not as potent as the observed by live PECs, but was significantly greater than that observed for control fixed-PECs (p<0.05), thus demonstrating that cell-to-cell interactions are partially responsible for the observed proliferative suppression.

The role of soluble factors in PECs mediated suppression was also examined. In these experiments transwells were used for PECs:CD4⁺ co-culture. PECs were plated in the upper chamber in a ratio 1:4 with respect to the naive CD4⁺ cells, which were previously stimulated with anti-CD3/CD28 plate bound antibodies. It was found that, even in the absence of cell to cell contact, there was significant inhibition in the proliferation of anti-CD3/CD28 antibodies stimulated CD4⁺ cells (approximately 50% inhibition, FIG. 4b, p<0.05), strongly implicating a role for soluble factors in the Gr-1⁺ PEC mediated suppression of T cell proliferation.

PECs Expanded by LNnT-Dex Secrete a Different Profile of Cytokines Than Control PECs.

To determine if the PECs expanded by polyvalent LNnT-Dex produced soluble factors involved in suppressive activity, the peritoneal cells were harvested 2 h or 18 h after injection of LNnT-Dex or dextran and cultured then for 72 h. Supernatants were harvested and used for analysis of cytokines or other soluble factors. The results demonstrated that the pattern of cytokines spontaneously released by LNnT-Dex expanded or dextran expanded PECs was different. Notably PECs expanded by LNnT-Dex produced significantly lower levels of pro-inflammatory cytokines such as IL-1β, IL-12, IL-18 and IFN-γ than PECs recruited by dextran (Table II). In addition, PECs expanded by LNnT-Dex produced significantly greater quantities of IL-10 and TGF-β than dextran recruited PECs (Table III).

TABLE II

| Strain | | | | | |
|---|---|---|---|---|---|
| PECs' origin | | BALB/c | | C57BL/6 | |
| Cytokines | | Dextran | LNnT | Dextran | LNnT |
| IFN-γ | 2 h | 497 ± 35 | 22.6 ± 22* | ND | ND |
| | 18 h | 427 ± 78 | 68.8 ± 26* | ND | ND |
| IL-18 | 2 h | 2148 ± 146 | 418 ± 150* | 882 ± 112 | 195 ± 65* |
| | 18 h | 591 ± 89 | 365 ± 37* | 1247 ± 446 | 68.2 ± 30* |
| IL-12 | 2 h | 350 ± 66 | 43 ± 34* | 761 ± 227 | 250 ± 132* |
| | 18 h | 1883 ± 434 | 332 ± 123* | 1428 ± 39 | 429 ± 71* |
| IL-1β | 2 h | 253 ± 29 | 68 ± 11* | <20 | <20 |
| | 18 h | 241 ± 94 | <20* | 60 ± 10 | 31.5 ± 8* |

Spontaneous pro-inflammatory cytokine production by PECs recruited by LNnT or Dextran in BALB/c or C57BL16 mice. PECs were obtained at 2 h or 18 h after injection, adjusted to 5 × 10⁵ cells/ml and 1 ml added in 24-wells place. Supernatants were harvested 96 h later. Data are representative of 5 independent experiments, using 4 mice per group individually assayed. Data are shown as Mean ± SE. *p value <0.05 compared with control values. ND = Not determined.

TABLE III

| Strain | | | | | |
|---|---|---|---|---|---|
| PECs' origin | | BALB/c | | C57BL/6 | |
| Cytokines | | Dextran | LNnT | Dextran | LNnT |
| IL-10 | 2 h | 108 ± 20 | 337 ± 39* | 188.1 ± 37 | 438 ± 31* |
| | 18 h | 397 ± 32.5 | 1140 ± 327* | 255.8 ± 64 | 933 ± 65* |
| TGF-β | 2 h | 434 ± 10.7 | 607 ± 34* | 706.8 ± 22 | 995.8 ± 154 |
| | 18 h | <20 | 7000 ± 375* | 397 ± 33 | 1433 ± 409* |

Spontaneous production of anti-inflammatory cytokines by PECs recruited by LNnT or Dextran in BALB/c or C57BL/6 mice. PECs were obtained and cultured under the same conditions as described for Table I. Data are representative of 5 independent experiments, using 4 mice per group individually assayed. Data are shown as Mean ± SE. *p value <0.05 compared with control values.

PECs Expanded by LNnT-Dex Inhibit IFN-γ Production in Co-Cultures with Naive CD4⁺ T Cells While Enhancing the Production of IL-13.

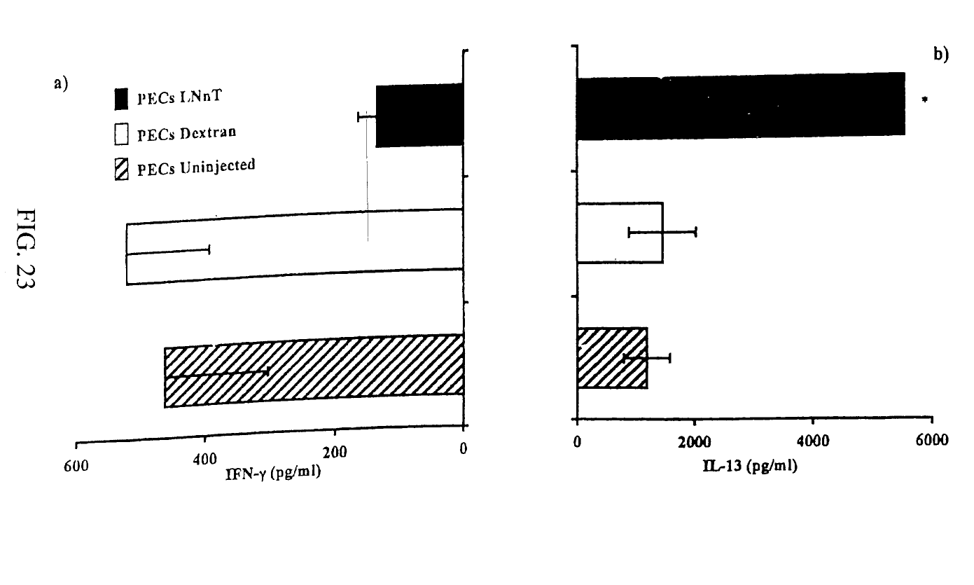

To exclude the possibility that the CD4⁺ cells in co-cultures had reduced proliferation due to cell death, cytokine production was measured in response to anti-CD3/CD28 stimulation in the presence of PECs. The results demonstrate that there was a significant decrease in IFN-γ production in supernatants from co-cultures containing PECs expanded by LNnT-Dex, compared to PECs from dextran or non-injected mice (FIG. 23a, p<0.05). Parallel testing in these LNnT-Dex-PECs:CD4⁺ supernatants revealed that levels of IL-13 were significantly elevated, compared to cultures containing dextran-recruited PECs (FIG. 23b, p<0.01). This, finding demonstrates that co-cultured CD4+ cells are alive and their cytokine profile is differentially regulated depending on the source of PECs.

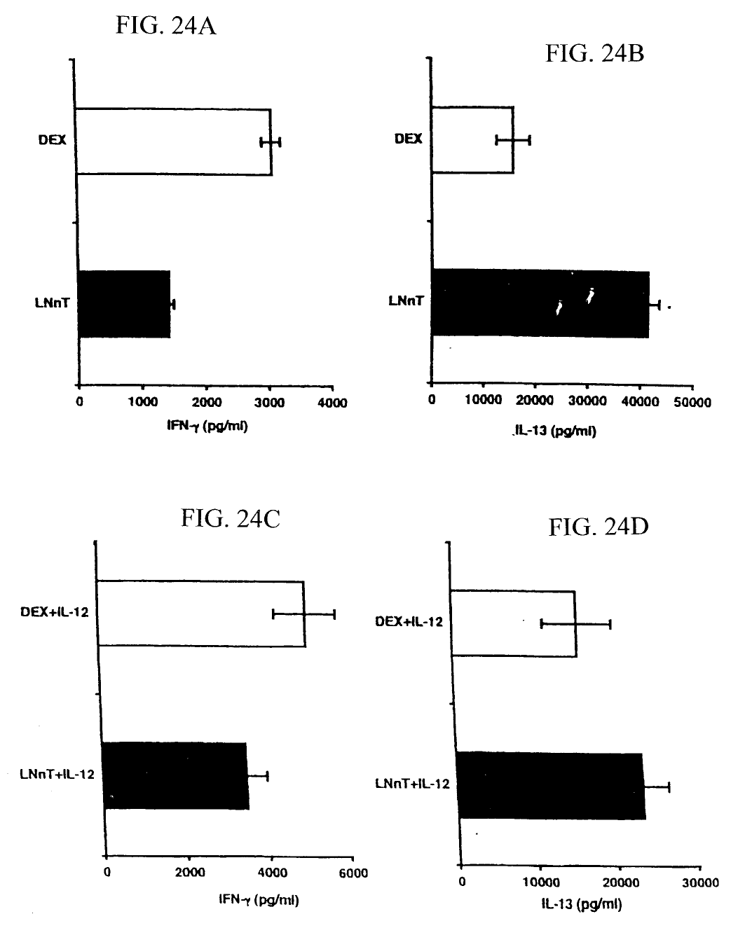

Co-cultured CD4⁺ cells were then examined to determine whether the cells would respond to secondary stimulation. PECs:CD4⁺ cells were co-cultured as previously described, but after 72 h in culture, cells were removed, washed and CD4⁺ cells re-purified. Purified CD4⁺ cells were then plated and incubated in RPMI for another 72 h (rested). CD4+ cells were then re-stimulated with anti-CD3/CD28 plate bound antibodies in the absence of PECs and their supernatants harvested 24 h later. Interestingly, rested and re-stimulated CD4 cells produced an IFN-γ profile in the secondary stimulation that was similar to those seen in the primary stimulation (FIG. 24a). Conversely, the production of IL-13 was enhanced in the secondary stimulation of CD4⁺ cells that came from primary co-cultures where the LNnT-Dex-PECs were present (FIG. 24b). These data indicate that CD4⁺ cells in co-cultures are alive and furthermore suggest that the majority of T cells isolated after primary co-culture with LNnT-Dex-PECs are Th2 committed, because the greatest amounts of IL-13 are secreted following secondary stimulation (Constant, et al., Annu. Rev. Immunol. 15:297, 1997). Other experiments showed that the addition of IL-12 could restore IFN-γ production in the co-culture LNnT-Dex/PECs:CD4⁺, confirming that CD4⁺ cells are alive and responding to external stimuli (FIG. 24c). In addition, IL-12 in the co-cultures diminished the previously highly detected IL-13 production (FIG. 24d), thereby suggesting that the absence, or low levels, of IL-12 in PECs expanded by LNnT-Dex could be a determining factor in the outcome of the response to anti-CD3/CD28 stimulation by naive CD4⁺ cells.

Blockade of IL-10 but not of TGF-β Abrogates Suppression Activity in PECs Recruited by LNnT-Dex.

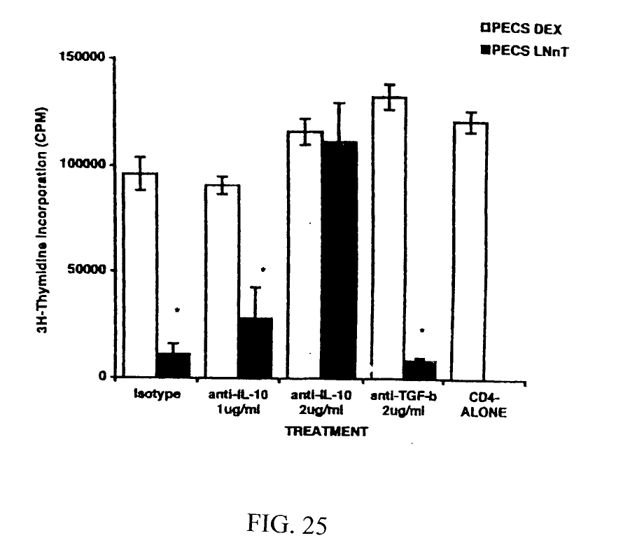

Because a significant inhibitory effect was shown in experiments where PECs:CD4⁺ co-cultures were performed with transwells, and because important differences in the release of IL-10 and TGF-β from PECs expanded by polyvalent LNnT-Dex versus those recruited by dextran were observed, neutralization experiments were conducted to determine if either of these molecules classically associated with inhibitory effects were involved in the suppressive activity. As shown in FIG. 25, neutralization of IL-10 with 2 μg/ml of blocking monoclonal antibodies resulted in a significant decrease (95%, p<0.01) in the ability of the PECs expanded by LNnT-Dex to induce their suppressive effect as measured by cellular proliferation. In contrast, neutralizing antibodies against TGF-β1,2 and 3 had no significant effect on cellular proliferation (FIG. 25). This shows that IL-10 plays a key role as a soluble factor in the suppressive activity associated with polyvalent LNnT-Dex expanded PECs.

In summary, this study demonstrated that as early as 2 h after injection of the neo-glycoconjugate, LNnT-Dex, the peritoneal cell population was significantly expanded in Gr-1⁺/CD11b⁺/F4/80⁺ cells. This population was maintained in vivo for at least 24 h post-injection of LNnT-Dex. The cells expanded by LNnT-Dex adoptively suppress a primary response to powerful stimuli such as anti-CD3 and anti-CD28 antibodies in naive CD4⁺ cells. Removal of Gr-1⁺ cells from this suppressor population renders the remaining PECs unable to suppress CD4⁺ cell proliferation in response to the same stimulation, even at the 1:2 ratio of PECs:CD4⁺ cell. These findings are evidence that the Gr-1⁺ cells are critical to the suppression activity. Similarly, it was demonstrated that this suppressive population can exert its action by both cell to cell contact (e.g., involving a membrane-associated factor) and through the release of soluble factors.

These experiments also demonstrated that CD4⁺ cells are still viable after rest and re-stimulation. Supernatants obtained from co-cultures where there was an inhibition of 90% in the proliferative response of CD4⁺ cells, contained high levels of cytokines, indicating that the cells were alive. Furthermore, when CD4⁺ cells in co-cultures were separated and given a secondary stimulation they maintained the profile of cytokine production seen in the initial co-cultures, that is, lower levels of IFN-γ and higher production of IL-13 compared with their respective controls. Together, these data support the fact that CD4⁺ cells remain viable in the presence of PECs recruited by LNnT-Dex.

Surprisingly, the blockade of TGF-β, a cytokine known to induce suppression in activated T cells (29, 35) did not restore the CD4⁺ proliferative response. In contrast to TGF-β, IL-10 was a major regulatory factor involved in the suppressive activity of PECs recruited by LNnT-Dex. As shown in the blocking Ab experiments, blockade of IL-10 was enough to restore the proliferative response of CD4⁺ cells co-cultured with PECs recruited by LNnT-Dex. IL-10 is a potent suppressor of cell mediated immune responses (involved in restricting cellular proliferation in S. mansoni infected individuals. This correlates with results which indicate that some of the complex carbohydrates structurally related to S. mansoni are critical in the down-regulation of the early events that favor the development of a Th1-type response. Another significant biological function of IL-10 is inhibition of both IFN-γ secretion by T cells and NO production in activated macrophages. Taken together, PECs expanded by multivalent LNnT-Dex produce two key cytokines with suppressive or anti-inflammatory activity, IL-10 and TGF-β, which are capable of counterbalancing the pro-inflammatory cytokines IL-1β, IL-12, IL-18 and IFN-γ.

The findings are interesting in light of the fact that other groups have identified similar populations of suppressor cells (Gr-1⁺/CD11b⁺) recruited by virus inoculation (Bronte, et al., J. Immunol. 161, 5313, 1998) or by tumor implantation (Bronte, et al. J. Immunol., 162:5728, 1999). In either case, the suppressor cell populations are expanded approximately 10 days post-challenge compared with the 2 hours seen in the system described herein. The rapid and local responses to the polysaccharide LNnT-Dex reported indicate that these sugars can be used therapeutically when an effective and fast anti-inflammatory response is needed, or as a potential adjuvant to induce Th2 responses later in disease progression.

### Example 6

### NOD-Model of Insulin-Dependent Diabetes Mellitus

In the NOD-Model, mice develop pancreatitis starting around 4 weeks of age, and 75% of the female mice develop Insulin-dependent diabetes mellitus (IDDM) between 25-30 weeks of age. While some interventions administered between 4-6 weeks of age have been found to be effective in preventing onset of the disease in these mice, few interventions initiated after 12 weeks are effective.

In this example, age matched female NOD mice were allowed to progress until 22 weeks of age. Blood sugars were examined, and found to be elevated at this age, but not technically diabetic. Mice were then given a single IP injection of LNn-T(25)-Dex or dextran (100 μg). Eight weeks later, mice were observed for symptoms and blood glucose levels were measured by standard methods.

The mice injected with dextran were completely wet, and the bedding soaked, indicating the presence of large amounts of dilute urine. In contrast, the mice injected with LNn-T-Dex were dry and the bedding was dry. The blood glucose levels in mg/dL for the mice are shown in Table IV.

TABLE VI

| Treatment | Mouse 1 | Mouse 2 | Mouse 3 | Mouse 4 |
|---|---|---|---|---|
| DEX | 796 | 732 | 191 | 271 |
| LNnT | 190 | 195 | 149 | 144 |

These results demonstrate that LNnT is effective in lowering the blood glucose levels in a art-recognized animal model of IDDM.

### Example 7

### SCID T Cell Transfer Model of IBD

In this example, one million wild-type naïve (CD45Rb^{high}) T cells were injected into SCID mice. The onset of inflammatory bowel disease (IBD) occurs 3-5 weeks following cell transfer, and visual symptoms (diarrhea, weight loss, prolapsed rectum) occur from 8-12 weeks post transfer. Animals were treated one week after cell transfer with IP injection of 100 μg of LNnt(35)-Dex or Dextran, followed by weekly injections. Experiments were terminated when mice demonstrated serious diarrhea or prolapsed rectum. Animals were necropsied, examined for gross lesions, and the tissues were examined using standard histological methods.

In one experiment, 75% (¾) of the mice treated with LNnT35-Dex were demonstrated no diarrhea or prolapsed rectum. In a second experiment, 100% (4/4) of the LNnT35-Dex treated mice were free of IBD symptoms. These results demonstrate that administration of multivalent LNnt is effective in preventing onset of IBD in an art-recognized model of IBD.

### Example 8

In this example, the effect of multivalent LNnt on TH1 severe combined immunodeficient (SCID) mouse model which mimics human Crohn's disease was further studied. SCID mice develop disease shortly after transfer of CD45RB hi CD4 wild-type T cells, and disease is accompanied by increases in T cell expression of interferon (IFN)-γ and tumor necrosis factor (TNF)-α, a profile believed to partially responsible for the lack of immunological tolerance and resulting colon inflammation (Powerie, et al., Int. Immunol. 5:1461-71 (1993); Morrissey, et al., J. Exp. Med. 178:237-44 (1993); and Powerie et al., Immunity 1: 553-62 (1994)).

To determine if LNnT would have an impact on the development of colitis, we administered LNnT-dextran conjugates to CD45RB hi CD4 T cell reconstituted SCID mice via intraperitoneal injection (IP), and monitored the development of the disease.

Mice

Donor six to ten week old BALB/c and recipient C.B-17 SCID mice were purchased from Taconic Farms (Germantown, N.Y.) or bred and housed at Harvard Institutes of Medicine animal care facilities according to institutional guidelines.

Donor T Cells

Single cell suspensions were prepared from spleens of BALB/c mice. Red blood cells were lysed using Boyle's solution (GIBCO) and the remaining white blood cells washed in RPMU 1640 (RPMI, GIBCO BRL, Life Technologies, Grand Island, N.Y.) supplemented with 2 mM-glutamine, 100 U/ml penicillin, 100 μg/ml streptomycin (Sigma Chemical Co., St. Louis Mo.), 5×10⁻⁵ M 2-β-mercaptoethanol (GIBCO) and 10% fetal bovine serum (FBS, Hyclone, Logan, Utah). Thy 1 positive cells were enriched using MACS (MidiMACS, Miltenyi Biotec, Germany) columns. Recovered cells were double stained with CD45RB Fc and CD4 PE monoclonal antibodies (PharMIngen, San Diego, Calif.) each at 1:100 dilution. Cells were sorted on a MoFlo high Speed Cell Sorter (Cytomation Inc., Fort Collins Colo.) and the 33% highest expressing CD45RB/CD4 T cells collected, resulting in greater than 97% purity. T cells were washed twice in Dulbecco's Phosphate-Buffered Saline (PBS, Life Technologies), adjusted to 7×10⁵−1×10⁶ (400 μl volume) and injected into the lateral tail vein of SCID mice.

LNnT-Dex Glycoconjugates

Lacto-N-neotraose (LNnT) conjugated to a 40 kDa dextran backbone was supplied by Neose Technologies Inc. (Horsham, Pa.). The glycoconjugates contained on average 25 or 35 LNnT residues per molecule of dextran (LNnT25-dex and LNnT35-dex, respectively). One-hundred to 150 μg of glycoconjugates or dextran were diluted in phosphate-buffered saline (PBS) and administered IP to T cell reconstituted SCID mice weekly, or once every two weeks. In experiments to determine if treatment with LNnT conjugates could prevent or diminish disease, treatment was initiated at day 1 or 2 post cell transfer. Treatment was initiated on day 16 post transfer in experiments examining the effect of LNnT conjugate on mice with early disease.

Disease Monitoring

All groups were weighed and examined weekly for signs of diarrhea, and sacrificed when soft stools were evident for one week in at least 75% of the control group. Final weight, colon thickening, rectal prolapse, stool consistency and splenic enlargement were assessed.

Histopathology

Colons were fixed in Bouin's Fixation Fluid (VSR, West Chester, Pa.) overnight and then in 10% Buffered Formaldehyde (Ricca Chemical Co., Arlington, Tex.). Paraffin embedded sections (6 um thickness) were stained with hematoxylin and eosin (H+E). Colonic damage was assessed in a blinded fashion and scored ranging from zero (0) for a normal colon to three (3) indicating the highest level of colon pathology.

Splenic CD4+ T Cell and Lymph Node Cell Preparation

Total spleen cells were prepared as described (Velupillai et al.i (1997), supra). CD4 T cells were enriched using MACS CD4 beads and columns (Miltenyi). One milliliter of cells/week (1×10⁶/ml) were plated in 24 well plates (Costar, Cambridge, Mass.) precoated with 1 ig/ml αCD3 monoclonal antibody (PharMingen), and then 2 μg/ml αCD28 (PharMingen) was added. Cultures were incubated in a humidified incubator containing 5% CO₂ at 37° C. Cytokine production was assessed by intracellular staining of cells cultured for 18 hours, or by ELISA of 72 hour culture supernatants. Mesenteric lymph nodes (LN) and Peyers' patches were teased apart and filtered through a 70 um nylon strainer (Becton Dickinson Labware, Franklin Lakes, N.J.), washed in RPMI (GIBCO) with supplements and cultured as for spleen cells.

Flow Cytometric Analysis and Intracytoplasmic Cytokine Staining

Surface phenotypes of freshly isolated spleen or LN cells were stained with CyChrome conjugated αCD4 (PharMingen), PE conjugated α4β7 (LPAM-I), αIELβ7 (CD103), and CCR5 monoclonal antibodies (PharMingen), or appropriate IgG isotype controls.

Briefly, cells were resuspended at 2-5×10⁵ cells in 100 μl RPMI with supplements, blocked with FcγIII/IIR antibody (CD16/CD32, PharMingen), and then incubated with fluorochrome labeled monoclonal antibodies for 30 minutes on ice. Cells were washed with Hanks' Balanced Salts Solution (HBSS, GIBCO) containing 2% FBS (Hyclone), and then resuspended in PBS and analyzed on a FACSCalibur (Becton Dickinson). We routinely acquired 5×10⁴ events for spleen and LN cells from reconstituted SCID mice, and 10⁴ events for other cell preparations. Intracellular (IC) staining was performed using Cytofix/Cytoperm Plus™ (with Golgi Stop™) Kit (PharMIngen) with Golgi Stop (brefeldin A) added for 5 hours after 18 hour cell culture. Cells were then surface stained for CD4 as described above, fixed, permeabilized and stained for cytokines using PE conjugated monoclonal antibodies for IFN-γ, TNF-α, or IL-10 (PharMingen). Events were acquired as described and the percentage positive cells analyzed using Cell Quest Software (Becton Dickinson).

Enzyme Linked Immunosorbent Assay (ELISA)

Quantitative ELISA assays for IL-10, Il-4 (using antibodies and cytokines from PharMingen) and TGF-β (antibodies and cytokines from R&D Systems, Inc., Minneapolis, Minn.) were performed using 72 hour supernatants from stimulated splenic T cell cultures.

Statistical Analyses

Data analyses used Unpaired Student's T Test with a significant value of P<0.05.

Results

LNnT-Dex Prevents Colitis in T Cell Reconstituted SCID Mice

Six to 12 weeks post-T cell transfer, SCID mice consistently develop diarrhea, thus providing an over parameter of colitis. Rectal prolapse was also noted in some experiments reaching diameters of up to 7 mm. Overall, prolapse was noted in 32% of dextran treated mice compared to 5% in LNnT35-dex treated mice and 0% in LNnT25-dex treated mice.

Preliminary investigations demonstrated that 100 μg or higher doses of glycoconjugates prevented colitis. IP administration of glycoconjugates weekly or on alternate weeks offered significant protection against disease (Table V).

TABLE V
LNnT-25-Dextran glycoconjugates prevented colitis
in reconstituted SCID mice.

| | | Number of Subjects | Percent of subjects |
|---|---|---|---|
| Treatment | Number of Trials | with Diarrhea | non-colitic |
| Dextran (100 μg) | 2 | 6/7 | 14 |
| Weekly | | | |
| LNnT-35 (100 μg) | 2 | 1/8 | 88 |
| Weekly | | | |
| Dextran (100 μg) | 1 | 3/4 | 25 |
| 1 of 2 weeks | | | |
| LNnT-25 (100 μg) | 1 | 0/4 | 100 |
| 1 of 2 weeks | | | |
| Dextran (150 μg) | 3 | 11/12 | 8 |
| Day 16, then weekly | | | |
| LNnT-25 (150 μg) | 3 | 2/12 | 83 |
| Day 16, then weekly | | | |

SCID mice were transplanted with CD45Rb^{hi}/CD4⁺T lymphocytes from BALB/c mice as described. Groups of 3 to 4 mice were treated with LNnT-Dextran glycoconjugates using the regimens indicated. Mice were sacrificed when dextran groups presented with diarrhea.

Treatment with LNnT-Dex effected cellular recruitment as dextran treated SCID mice had a five-fold increase in the number of splenocytes and recoverable cells from colleganase digested colons compared to wild-type mice (Table VI). In comparison, LNnT25-dex treated SCID mice only had a two-fold increase in the same cell population.

TABLE VI
LNnT-25-dextran results in decreased number of spleen cells

| | Spleen Cells/Mouse × 10⁶ | |
|---|---|---|
| Treatment | Average Cell Numbers | Number of Mice |
| Dextran SCID | 79.3 | 20 |
| LNnT-35-Dextran SCID | 22.1 | 10 |
| LNnT-25-Dextran SCID | 23.7 | 11 |
| Normal BALB/c | 70.1 | 9 |

Values represent the average numbers of spleen cells per mouse in groups as indicated, over 3-5 experiments. * denotes statistically significant values by the Unpaired Students' T test (P < 0.05).

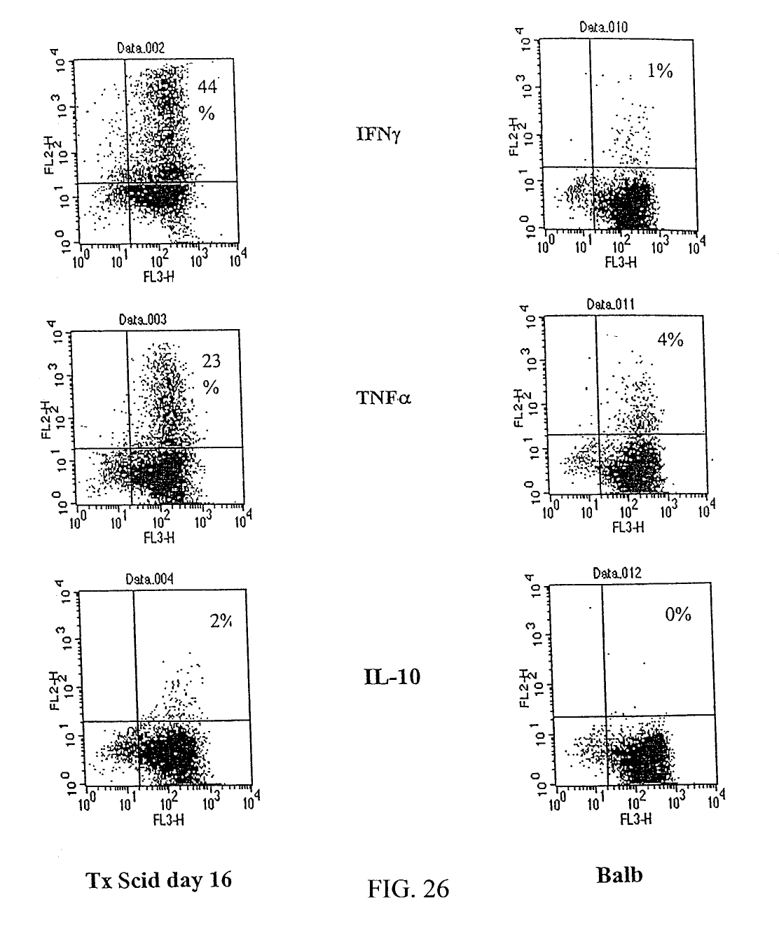

At day 16 post T cell transfer, untreated mice appeared normal. However, intracellular staining of stimulated splenic T cells from these mice showed elevated levels of IFN-γ and TNF-α compared with cells from BALB/c mice (FIG. 26). These two cytokines were also elevated in lymph node T cells. Increased levels of these cytokines have been associated with disease, thus, mice 16 days post-transfer were selected to initiate LNnT-dex treatment in experiments to determine whether glycoconjugate treatment alters outcome in mice with early disease. Surprisingly, administration of LNnT-dex to mice at day 16 was effective in halting disease progression and preventing diarrhea and other symptoms of colitis (Table V).

LNnT-Dex Prevents Cellular Infiltration and Colon Destruction

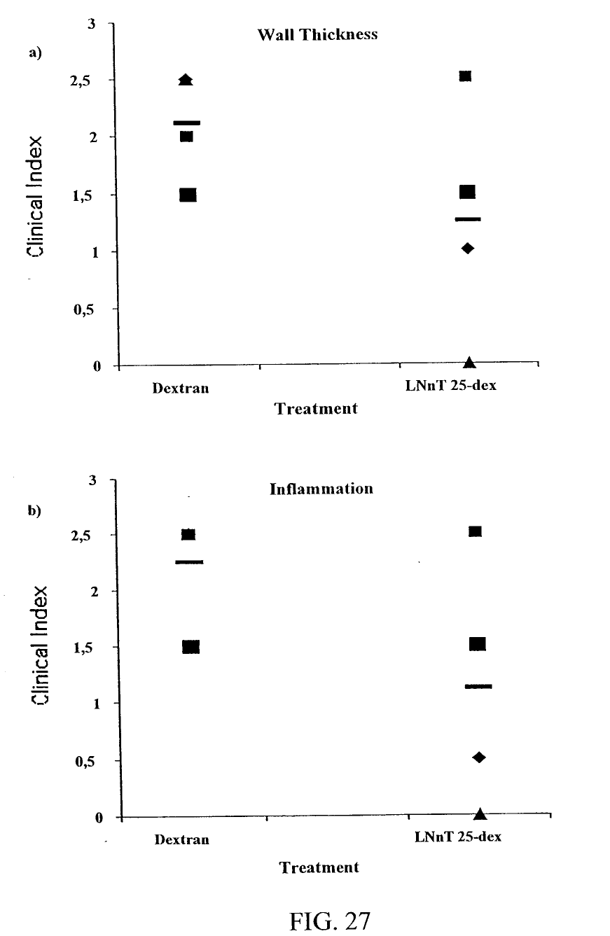

Histophathologic analyses of colon sections revealed that treatment of T cell reconstituted SCID mice with dextran or PBS had similar levels of colon thickening, loss of architecture and cellular infiltration compared with naïve wild-type mice. In contrast, mice treated with LNnT25-dex or LNnT35-dex from the start of reconstitution or from day 16, had significantly reduced colon thickening and cellular infiltration, and often there was no apparent difference in these tissues compared to wild-type colons (FIGS. 27a and b).

Abrogation of Chemokine Receptor CCR5 After Glycoconjugate Treatment

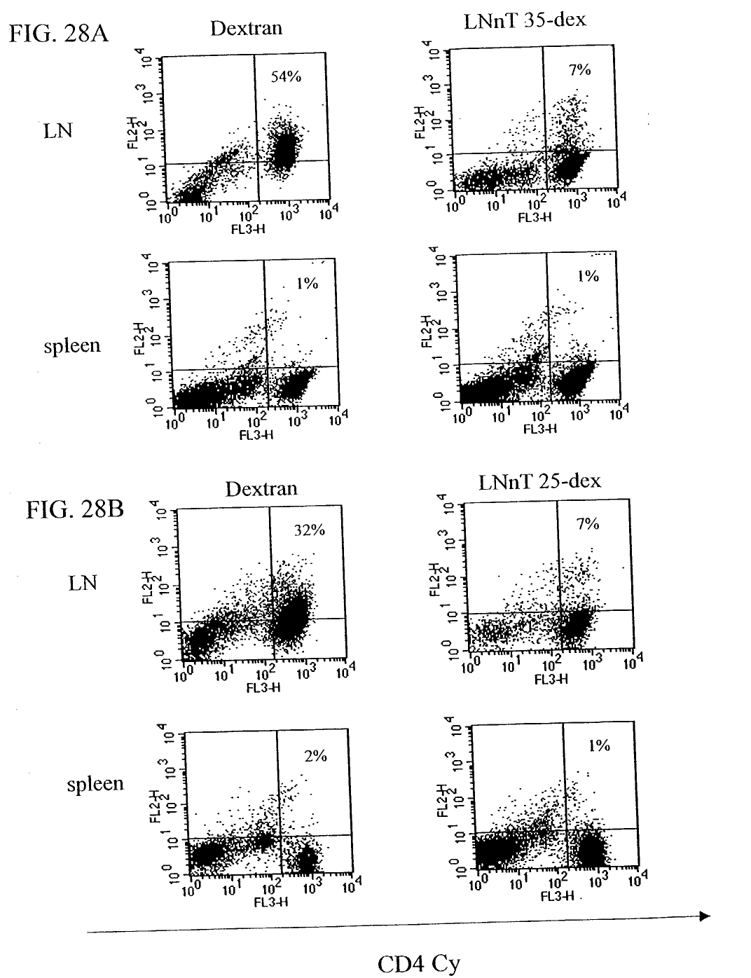

CCR5 plays a role in the recruitment of cells to sites of inflammation. Expression of CCR5rwas examined and minimal expression on splenic T cells was found forall groups. Elevated expression of CCR5r was detected on lymph node T cells from mice treated with dextran, suggesting that draining lymph node cells were the relevant population to analyze (FIGS. 28a and b). Interestingly, lymph node T cells from mice treated with LNnT-dex did not demonstrate an elevated expression of CCR5r as was seen in cells from control (dextran) treated mice. Thus, IP administration of LNnT-dex effects a reduction in CCR5 expression in GALT T cells. LNnT-dex mice were protected from colitis suggesting that reduction in CCR5r expression is beneficial.

Integrin Expression is Reduced in Response to Glycoconjugate Treatment

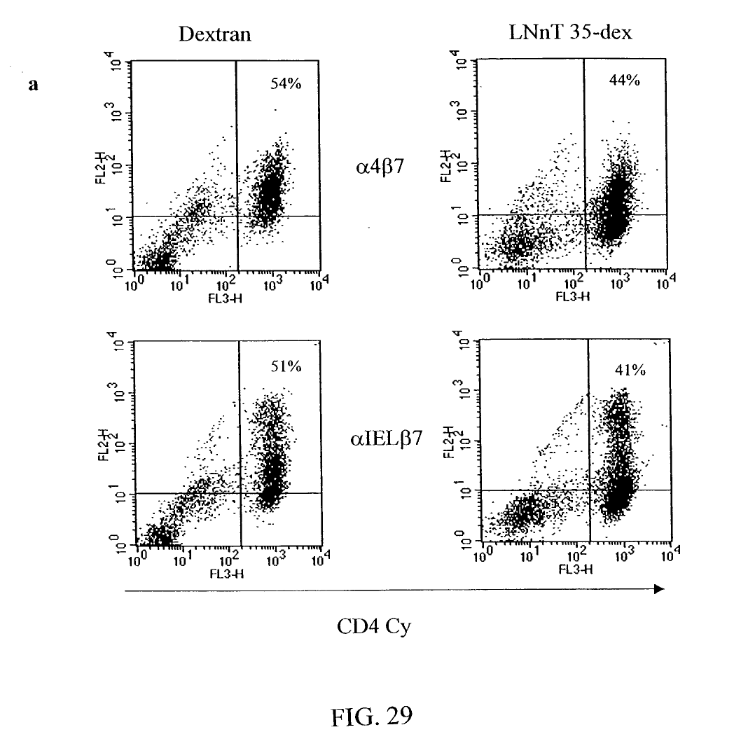

Integrin α4β7 participates in trafficking of T cells to mucosal tissues (Kilshaw, et al., Eur. J. Immunol. 21:2591-7 (1991); Wagner et al., Nature 382:366-70 (1996); Butcher et al. Science 272:60-6 (1996); Berlin et al., Cell 74:185 (1993)). Accordingly, T cells were examined to determine whether there was a correlation between elevated CCR5r expression and increased cellular migration to the colons of colitic mice. T cells from lymph nodes and Peyers' patches of all groups were analyzed, and it was determined that T cells from colitic, PBS or dextran treated control mice had elevated expression of α4β7 as compared to T cells from non-colitic mice treated with LNnT-dex (25 or 35) (FIG. 29).

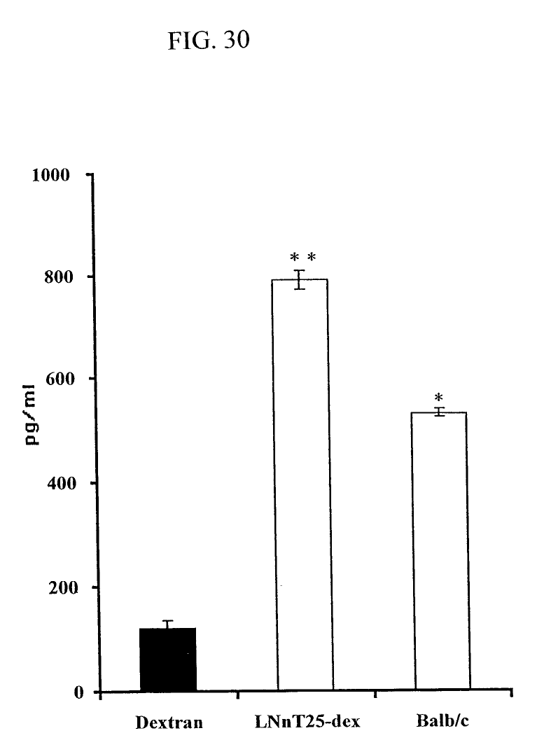

The levels of αIELβ7 expression in T cells were also examined. High αIELβ7 expression on T cells contributes to the retention of these cells at the site of inflammation (Lukviksson, et al., J. Immunol. 162:4975-82 (1999); Cepek, et al., J. Immunol. 150:3459-70 (1993); Cepek et al. nature 372:190-3 (1994); Schon, et al., J. Immunol. 162:6641-9 (1999)). Hence, an elevated expression of this molecule was observed on T cells obtained from regional lymphoid tissue of colitic mice that had active cellular infiltration and tissue destruction (FIG. 30). As with CCR4 expression, differences in α4β7 and αIELβ7 levels were not observed in spleen cells suggesting that expression is specific for draining lymph nodes and lymphoid tissues ant that LNNT-dex treatment down-regulates expression of both molecules at these sites.

Enhance Production of Th2 Cytokines in Glycoconjugate Treated Mice

The correlation of the protective effect of LNnT-dex treatment with alterations in cytokine production was also investigates. It was found that there was an increase in IL-4 production in culture supernatants from splenic T cells from mice treated with LNnT-dex (25 or 35) compared to cells from dextran treated mice (FIG. 30). The levels of TGF-β were similar for cells from LNnT25-dex mice or dextran mice. The levels for IL-10, TNF-α, and IFN-γ varied between experiments.

Discussion

These data demonstrate that, surprisingly, treatment with multivalent forms of LNnT (e.g., LNnT25-dex, LNnT35-dex) prevented disease onset using the CD45RB hi CD4 cell reconstituted SCID mouse model of colitis, dramatically reducing symptoms (increased colon size, cellular infiltration in colons, splenomegaly, rectal prolapse and diarrhea) compared to mice treated with dextran. Further, treatment with multivalent LNnT reversed early onset of disease when administered at day 16 post reconstitution.

These data also demonstrate that expression of CCR5 was suppressed in the GALT of mice treated with multivalent LNnT. Other studies have shown that downregulation of CCR5 expression correlated with decreases in tissue inflammation in colons (Agace, et al., Eur J. Immunol. 30:819-26, 2000) and kidneys (Mack et al, J. Immunol. 166:4697-704, 2001). CCR5 expression has also been shown to correlate with a pro-inflammatory milieu and IL-12 increases expression of CCR5r on LN cells (Iwasaki et al., Eur. J. Immunol., 31:2411-20, 2001). In regard to colitis, Andres et al. showed that CCR5−/− mice have less severe colitis following administration of a pro-inflammatory agent (J. Immuno. 164:6303-12, 2000). Accordingly, without being bound by any one theory, the reduction of cell surface expression of CCR5 following treatment with multivalent LNnT appears to be one mechanism leading to reduced pathology.

In addition to CCR5, disease amelioration in experimental colitis models has also been attributed to decreased trafficking and localization of cells into the colon due to decreased expression of beta 7 integrin molecules (Picarella et al., J. Immunol. 158:2099-106; Hesterberg et al., Gastroenterology 111:1378-80, 1996). In the present study, a decrease in integrin expression was observed on GALT tissues and in LN cells from mice treated with multivalent LNnT as compared to mice treated with dextran. Interestingly, however, no significant differences in integrin expression were observed in spleen cells. These data demonstrate a utility for multivalent LNnT in reducing disease-associated pathology by altering cell trafficking and retention.

In addition to alterations in homing and adhesion molecules, cytokine levels were also examined in treated and untreated mice. In general, the ability to correlate cytokine changes in various models of IBD has been difficult, and data within the same studies are often conflicting (Dohi et al., Gastroenterology 119:724-33, 2000). Except for levels of IL-4, which were elevated in splenic T cells of mice treated with multivalent LNnT, these data did not detect a correlation between changes in Th1 and Th2 cytokine expression and disease severity. A role for T cell IL-4 has been shown in a study of colitic rats (Hogaboam et al., J. clin. Invest. 100:2766-76, 1997), and in CCR5−/− mice reduced colonic damage was associated with increased IL-4 mRNA (Andres et al., supra). However, even though it was recently shown that LNnT-dex drives a Th2 cytokine bias in vivo and in vitro (Atochina et al., J. Immunol. 167:4293-302, 2001; Terazas, et al., J. Immunol. 167:5294-303), the data presented here indicates that this is not the sole mechanism of glycoconjugate action. Rather, these data strongly support novel roles for multivalent LNnT in reducing regional lymph node cell expansion simultaneously with decreased leukocyte homing to the intestine, two related mechanisms that likely contribute to disease prevention.

Current therapies for inflammatory bowel disease utilize immunosuppressive steroids, cyclosporin and recently, anti-TNF-α antibodies. The data presented here indicates that immunomodulatory, multivalent forms of LNnT represent a new class of drug for treating Th1 type autoimmune diseases that is expected to have fewer side effects than existing treatments. Further, based these glycoconjugates can also be administered to children as a vaccine that will bias the immune system of most individuals thereby preventing or ameliorating the development of Th1 type autoimmune diseases.

Equivalents

Those skilled in the art will recognize, or be able to ascertain using no more than routine experimentation, many equivalents to the specific embodiments of the invention described herein. Such equivalents are intended to be encompassed by the following claims.

We claim:

1. A method of treating a Th1-associated autoimmune disease in a subject comprising:administering to the subject having the Th1-associated autoimmune disease a multivalent conjugate comprising multiple lacto-N-neotetraose (LNnT)-containing oligosaccharides crosslinked to a carrier molecule, such that the Th1-associated autoimmune disease is treated in the subject.

2. A method of preventing a Th1-associated autoimmune disease in a subject comprising administering to the subject a multivalent conjugate comprising multiple LNnT-containing oligosaccharides crosslinked to a carrier molecule such that the Th1-associated autoimmune disease is prevented in the subject.

3. The method of claims 1 or 2 wherein the carrier is a protein.

4. The method of claim 3, wherein the protein is human serum albumin.

5. The method of claims 1 or 2, wherein the carrier is a carbohydrate polymer.

6. The method of claim 5, wherein carbohydrate polymer is dextran.

7. The method of claim 5, wherein the carbohydrate polymer has a molecular weight of about 5,000 to 100,000 daltons.

8. The method of claim 7, wherein the carbohydrate polymer has a molecular weight of about 10,000 to 40,000 daltons.

9. The method of claims 1 or 2 wherein the multivalent conjugatehas about 2 to 200 LNnT-containing oligosaccharide molecules per carrier molecule.

10. The method of claim 9, wherein the multivalent conjugate has about 10 to 100 LNnT-containing oligosaccharide molecules per carrier molecule.

11. The method of claim 9, wherein the multivalent conjugate has about 20 to 50 LNnT-containing oligosaccharide molecules per carrier molecule.

12. The method of claim 9, wherein the multivalent conjugate is selected from the group comprising LNnT25-dex, LNnT35-dex, LNnT45-dex and LNnT50-dex.

13. The method of claims 1 or 2, wherein the multivalent conjugate is administered intraperitoneally.

14. The method of claim 1 or 2, wherein the multivalent conjugate is administered intravenously.

15. The method of claims 1 or 2, wherein the autoimmune disease is inflammatory bowel disease.

16. The method of claim 1 or 2, wherein the autoimmune disease is Type 1 diabetes.

17. The method of claim 1 or 2, wherein the autoimmune disease is arthritis.

18. The method of claim 1 or 2, wherein the autoimmune disease is psoriasis.

19. The method of claim 1 or 2, wherein the autoimmune disease is Crohn's disease.

20. A method of treating or preventing shock in a subject comprising: administering to the subject having or at risk of developing shock a multivalent conjugate comprising multiple LNnT-containing oligosaccharides crosslinked to a carrier molecule, such that shock is treated or prevented in the subject.

21. A method inhibiting inflammation in a subject comprising administering to the subject a multivalent conjugate comprising multiple LNnT-containing oligosaccharides crosslinked to a carrier molecule, such that inflammation is inhibited in the subject.

22. A method of expanding a Gr-1+ cell population in a subject comprising administering to the subject an effective amount of a multivalent conjugate comprising multiple LNnT-containing oligosaccharides crosslinked to a carrier molecule.

23. A method of inhibiting induction of Gr1+, CD11b+ suppressor cells in a subject comprising administering to the subject an effective amount of a monovalent LNnT-containing oligosaccharide.

24. The method of claim 20, wherein the subject is suffering from cancer.
